(19) United States
(12) Patent Application Publication
(10) Pub. No.: US 2025/0320519 A1
(43) Pub. Date: Oct. 16, 2025

(54) SYNTHETIC CIRCULAR RNA COMPOSITIONS AND METHODS OF USE THEREOF

(71) Applicant: SHANGHAI CIRCODE BIOMED CO., LTD., Shanghai (CN)

(72) Inventors: Yun YANG, Shanghai (CN); Zefeng WANG, Shanghai (CN)

(21) Appl. No.: 18/868,935

(22) PCT Filed: May 29, 2023
(86) PCT No.: PCT/CN2023/096813

§ 371 Date: Nov. 25, 2024

(30) Foreign Application Priority Data
May 30, 2022 (WO) PCT/CN2022/095949

(51) Int. Cl. C12N 15/85; A61K 48/00

(52) U.S. Cl. CPC C12N 15/85; A61K 48/00; C12N 2840/203

(57) ABSTRACT

The present disclosure relates to compositions, methods, processes, kits and devices for the selection, design, preparation, manufacture, formulation, and/or use of a polynucleotide having an Internal Ribosome Entry Site (IRES) sequence, an IRES-like sequence or a combination thereof. In particular, the present disclosure relates to compositions, methods, processes, kits and devices for the selection, design, preparation, manufacture, formulation, and/or use of a circular polynucleotide (e.g., a circular RNA). The present disclosure also relates to a method of improving expression, functional stability, immunogenicity, ease of manufacturing and/or half-life of a therapeutic product encoded by the circular RNA.

### 1. RELATED APPLICATIONS

The application claims priority to, and the benefit of PCT Application No. PCT/CN2022/095949, filed on May 30, 2022, the contents of which are incorporated herein by reference in their entirety.

### 2. INCORPORATION BY REFERENCE OF SEQUENCE LISTING

The contents of the electronic sequence listing (Family 03_sql_0529.xml; Size: 12,831,515 bytes; and Date of Creation: May 29, 2023) are herein incorporated by reference in its entirety.

### 3. FIELD

The present disclosure relates to compositions of matters, methods, processes, kits, and devices for the selection, design, preparation, manufacture, formulation, and/or use of a polynucleotide having an Internal Ribosome Entry Site (IRES) sequence, an IRES-like sequence, or a combination thereof. The present disclosure also relates to compositions of matters, methods, processes, kits, and devices for the selection, design, preparation, manufacture, formulation, and/or use of a circular polynucleotide (e.g., a circular RNA) comprising the IRES, sequence, IRES-like sequence, or a combination thereof. In addition, the present disclosure relates to methods of improving expression, functional stability, immunogenicity, manufacturing, and/or half-life of a therapeutic product encoded by the circular RNA.

### 4. BACKGROUND

Gene therapy and genetic vaccination provide highly specific and individualized therapy options for a large variety of diseases such as inherited genetic diseases, autoimmune diseases, cancer, and inflammatory diseases. In the context of gene therapy and genetic vaccination, DNA as well as RNA may be used as nucleic acid molecules for administration. While DNA therapies are stable and easily manipulated, there is a risk of undesirable consequences, such as genomic integration and generation of anti-DNA antibodies. Further, there may be limited expression of the encoded protein due the dependency on the presence of specific transcription factors, which regulate DNA transcription. In the absence of such factors, DNA transcription is hindered, which in turn results in low levels of translated protein.

By using RNA instead of DNA genetic therapies, the risk of undesirable consequences, such as genomic integration and generation of anti-DNA antibodies, is minimized or avoided. In particular, circular RNA is useful in the design and production of stable forms of RNA. Circular RNA is also useful for in vivo applications, especially in areas of RNA-based control of gene expression, protein manufacture, and therapeutics, including protein replacement therapy and vaccination.

Translation of circular RNA is facilitated by cap-independent translation. Thus, the design and selection of suitable cap-independent translation initiation elements is important for the control of protein expression. Internal ribosome entry site (IRES) elements are useful for cap-independent gene expression eukaryotic cells. However, naturally found IRES elements may 1) have a large length of nucleic acid residues, 2) contain complex secondary structures, and/or 3) be prone to host cell immune rejection, each of which is not preferable for in vivo applications, such as protein replacement therapies.

While numerous natural IRES sequences shown to promote cap-independent translation have been identified, the identification and development of novel IRES elements has been occasional and accidental. Natural IRES elements do not allow for fine-tuned control of protein expression and few systematic methodologies for prediction of functional novel IRES elements have been shown. Systematic approaches for the identification and development of synthetically generated IRES elements that are capable of providing efficient cap-independent protein expression have not been shown.

Therefore, there exists a need in the art for approaches and methodologies for developing synthetic IRES elements, and for methodologies in predicting and controlling the efficiency of protein expression from a synthetic IRES element. There also exists a need in the art for providing polynucleotides (e.g., circular RNA) having synthetic IRES elements that may be suitable for use as a medicament or vaccine, such as for application in gene therapy and/or genetic vaccination. The present disclosure addresses these unmet needs.

### 5. SUMMARY

Compositions of matters (e.g., synthetic Internal Ribosome Entry Site (IRES) sequences, IRES-like sequences, or combinations thereof, and circular RNAs) and methods are described for the selection, design, preparation, manufacture, formulation, and/or use of a polynucleotide having an IRES sequence, an IRES-like sequence, or a combination thereof, and a circular RNA, as described herein.

Provided herein is an RNA polynucleotide comprising a construct of Formula I:

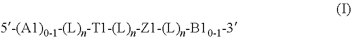

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence;
A1 and B1 are each independently a sequences capable of circularizing the RNA polynucleotide, or
A1 and B1 each independently comprise a nucleotide derivative capable of joining the 5′ end and the 3′ end via a 3′ to 5′ phosphodiester linkage for circularization of the RNA polynucleotide; and
n is an integer selected from 0 to 2.

Also provided herein is an RNA polynucleotide comprising a construct of Formula II:

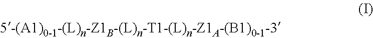

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1_{A} is a first portion of an expression sequence encoding a therapeutic product;
Z1_{B} is a second portion of the expression sequence encoding the therapeutic product;
each L is independently a linker sequence;
A1 and B1 are each independently a sequence capable of circularizing the RNA polynucleotide, or
A1 and B1 each independently comprise a nucleotide derivative capable of joining the 5′ end and the 3′ end via a 3′ to 5′ phosphodiester linkage for circularization of the RNA polynucleotide; and
n is an integer selected from 0 to 2.

Also provided herein is an RNA polynucleotide comprising a construct of Formula III:

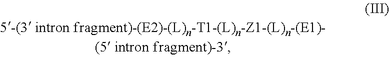

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence;
the 5′ intron fragment and the 3′ intron fragment are each a fragment of a group II intron, wherein the 5′ intron fragment is located on the 5′ side of the 3′ intron fragment in the group II intron;
the E1 is a 5′ adjacent exon fragment of the group II intron, which is ≥0 nucleotides in length;
the E2 is a 3′ adjacent exon fragment of the group II intron, which is ≥0 nucleotides in length; and
n is an integer selected from 0 to 2.

Also provided herein is an RNA polynucleotide comprising a construct of Formula IV:

5′-(3′ intron fragment)-(E2)-(L)ₙ-Z1_{B}-(L)ₙ-TI-(L)ₙ-Z1_{A-}-(L)ₙ-(E1)-(5′ intron fragment)-3′ (IV)

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1_{A} is a first portion of an expression sequence encoding a therapeutic product;
Z1_{B} is a second portion of the expression sequence encoding the therapeutic product;
each L is independently a linker sequence;
the 5′ intron fragment and the 3′ intron fragment are each a fragment of a group II intron, wherein the 5′ intron fragment is located on the 5′ side of the 3′ intron fragment in the group II intron;
the E1 is a 5′ adjacent exon fragment of the group II intron, which is ≥0 nucleotides in length;
the E2 is a 3′ adjacent exon fragment of the group II intron, which is ≥0 nucleotides in length; and
n is an integer selected from 0 to 2.

Also provided herein is an RNA polynucleotide comprising a construct of Formula V,

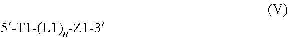

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence; and
n is an integer selected from 0 to 2.

Also provided herein is an RNA polynucleotide comprising an engineered translation initiation element (TI) comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341.

Also provided herein is an RNA polynucleotide comprising a construct of Formula I, Formula II, Formula III, Formula IV, or Formula V:

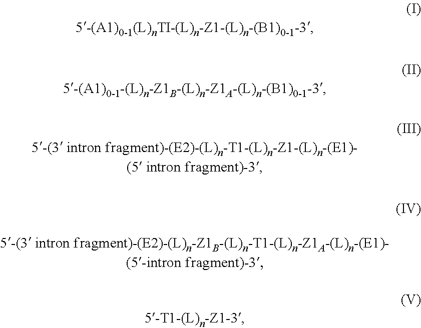

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
Z1_{A} is a first portion of an expression sequence encoding a therapeutic product;
Z1_{B} is a second portion of the expression sequence encoding the therapeutic product;
each L is independently a linker sequence;
A1 and B1 are each independently a sequences capable of circularizing the RNA polynucleotide, or
A1 and B1 each independently comprise a nucleotide derivative capable of joining the 5′ end and the 3′ end via a 3′ to 5′ phosphodiester linkage for circularization of the RNA polynucleotide;
the 5′ intron fragment and the 3′ intron fragment are each a fragment of a group II intron, wherein the 5′ intron fragment is located on the 5′ side of the 3′ intron fragment in the group II intron;
the E1 is a 5′ adjacent exon fragment of the group II intron, which is ≥0 nucleotides in length;
the E2 is a 3′ adjacent exon fragment of the group II intron, which is ≥0 nucleotides in length; and
n is an integer selected from 0 to 2.

Provided herein is a method for generating an Internal Ribosome Entry Site (IRES)-like polynucleotide sequence, the method comprising the steps of:
(a) generating a polynucleotide query sequence consisting of X nucleic acid residues in length, wherein X is an integer greater than or equal to 3;
(b) generating X−Y+1 number of overlapping polynucleotide fragment sequences within the polynucleotide query sequence,
wherein each polynucleotide fragment sequence consists of Y nucleic acid residues in length,
wherein the first position of each polynucleotide fragment sequence is n and the last position of the same polynucleotide fragment sequence is Y+n−1, and
wherein n represents each positive integer between 1 and X−Y+1;
(c) determining an enrichment score for each polynucleotide fragment sequence of (b);
(d) determining a numerical score for the polynucleotide query sequence by summing the enrichment scores of each polynucleotide fragment sequence of (c); and
(e) identifying the polynucleotide query sequence as an engineered IRES-like polynucleotide sequence according to a reference value.

Provided herein is a method of determining an enrichment score for a polynucleotide fragment sequence, comprising
i) generating an expression plasmid library, wherein each expression plasmid of the library comprises a different polynucleotide fragment sequence and a reporter gene;
ii) contacting a population of cells with the expression plasmid library;
iii) quantifying the expression level of the reporter gene corresponding to each expression plasmid of the expression plasmid library;
iv) dividing the total population of cells into a first population and a second population based on the protein expression levels of iii);
v) determining an enrichment score for the polynucleotide fragment sequence using the following system of equations:

$$\begin{matrix}Score = \frac{{f}_{1} - {f}_{2}}{\sqrt{( \frac{1}{{N}_{1}} + \frac{1}{{N}_{2}} ) P ( 1 - P )}} \\ P = \frac{{N}_{1} {f}_{1} - {N}_{2} {f}_{2}}{{N}_{1} + {N}_{2}}\end{matrix}$$

wherein f₁ is the frequency of the polynucleotide fragment sequence in the first population,
wherein f₂ is the frequency of the same polynucleotide fragment sequence in the second population,
wherein N₁ is the size of the first population, and
wherein N₂ is the size of the second population.

Provided herein is an RNA polynucleotide comprising an engineered translation initiation element (TI), wherein the TI comprises an Internal Ribosome Entry Site (IRES)-like polynucleotide sequence generated by the method of the present disclosure.

Provided herein is a polypeptide expressed by the RNA polynucleotide of the present disclosure, a DNA vector encoding or suitable for synthesizing the RNA polynucleotide of the present disclosure, a cell comprising the RNA polynucleotide, polypeptide, or DNA vector of the present disclosure, and a composition comprising the RNA polynucleotide, polypeptide, DNA vector, or cell of the present disclosure, and a pharmaceutically acceptable carrier, and a method of making the same.

Provided herein is a method of modulating the expression of a protein in a subject in need thereof, comprising administering to the subject a therapeutically effective amount of the RNA polynucleotide, polypeptide, DNA vector, or cell of the present disclosure.

Provided herein is a method of treating or preventing a disease or disorder in a subject in need thereof, comprising administering to the subject a therapeutically effective amount of the RNA polynucleotide, polypeptide, DNA vector, or cell of the present disclosure.

Provided herein is use of the RNA polynucleotide, polypeptide, DNA vector, or cell of the present disclosure in the manufacture of a medicament for modulating the expression of a protein or treating or preventing a disease or disorder in a subject in need thereof.

Provided herein is an RNA polynucleotide, polypeptide, DNA vector, or cell of the present disclosure for modulating the expression of a protein or treating or preventing a disease or disorder in a subject in need thereof.

In some embodiments, nucleic acids (e.g., polynucleotides) and nucleic acid sequences disclosed herein may be codon-optimized (e.g., for the expression of the therapeutic product), for example, via any codon-optimization technique known in the art (see, e.g., Quax et al., 2015, Mol Cell 59: 149-161).

### 6. ILLUSTRATIVE EMBODIMENTS

### Embodiment 1

An RNA polynucleotide comprising a construct of Formula I:

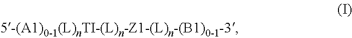

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence;
A1 and B1 are each independently a sequence capable of circularizing the RNA polynucleotide, or
A1 and B1 each independently comprise a nucleotide derivative capable of joining the 5′ end and the 3′ end via a 3′ to 5′ phosphodiester linkage for circularization of the RNA polynucleotide; and
n is an integer selected from 0 to 2.

### Embodiment 1-1

An RNA polynucleotide comprising a construct of Formula I-1:

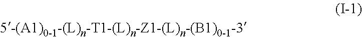

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence;
A1 and B1 are each independently a sequence capable of circularizing the RNA polynucleotide; and
n is an integer selected from 0 to 2.

### Embodiment 1-2

An RNA polynucleotide comprising a construct of Formula 1-2:

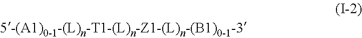

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence;
A1 and B1 each independently comprise a nucleotide derivative capable of joining the 5′ end and the 3′ end via a 3′ to 5′ phosphodiester linkage for circularization of the RNA polynucleotide; and
n is an integer selected from 0 to 2.

### Embodiment 2

An RNA polynucleotide comprising a construct of Formula II:

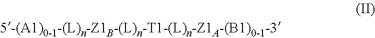

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1_{A} is a first portion of an expression sequence encoding a therapeutic product;
Z1_{B} is a second portion of the expression sequence encoding the therapeutic product;
each L is independently a linker sequence;
A1 and B1 are each independently a sequence capable of circularizing the RNA polynucleotide, or
A1 and B1 each independently comprise a nucleotide derivative capable of joining the 5′ end and the 3′ end via a 3′ to 5′ phosphodiester linkage for circularization of the RNA polynucleotide; and
n is an integer selected from 0 to 2.

### Embodiment 2-1

An RNA polynucleotide comprising a construct of Formula II-1:

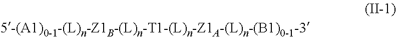

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1_{A} is a first portion of an expression sequence encoding a therapeutic product;
Z1_{B} is a second portion of the expression sequence encoding the therapeutic product;
each L is independently a linker sequence;
A1 and B1 are each independently a sequence capable of circularizing the RNA polynucleotide; and
n is an integer selected from 0 to 2.

### Embodiment 2-2

An RNA polynucleotide comprising a construct of Formula II-2:

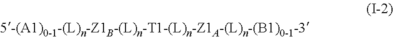

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1_{A} is a first portion of an expression sequence encoding a therapeutic product;
Z1_{B} is a second portion of the expression sequence encoding the therapeutic product;
each L is independently a linker sequence;
A1 and B1 each independently comprise a nucleotide derivative capable of joining the 5′ end and the 3′ end via a 3′ to 5′ phosphodiester linkage for circularization of the RNA polynucleotide; and
n is an integer selected from 0 to 2.

### Embodiment 3

An RNA polynucleotide comprising a construct of Formula III:

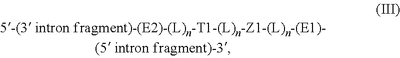

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence;
the 5′ intron fragment and the 3′ intron fragment are each a fragment of a group II intron, wherein the 5′ intron fragment is located on the 5′ side of the 3′ intron fragment in the group II intron;
the E1 is a 5′ adjacent exon fragment of the group II intron, which is ≥0 nucleotides in length;
the E2 is a 3′ adjacent exon fragment of the group II intron, which is ≥0 nucleotides in length; and
n is an integer selected from 0 to 2.

### Embodiment 4

An RNA polynucleotide comprising a construct of Formula IV:

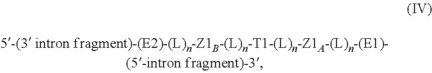

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1_{A} is a first portion of an expression sequence encoding a therapeutic product;
Z1_{B} is a second portion of the expression sequence encoding the therapeutic product;
each L is independently a linker sequence;
the 5′ intron fragment and the 3′ intron fragment are each a fragment of a group II intron, wherein the 5′ intron fragment is located on the 5′ side of the 3′ intron fragment in the group II intron;
the E1 is a 5′ adjacent exon fragment of the group II intron, which is ≥0 nucleotides in length;
the E2 is a 3′ adjacent exon fragment of the group II intron, which is ≥0 nucleotides in length; and
n is an integer selected from 0 to 2.

### Embodiment 5

The RNA polynucleotide of Embodiment 3 or 4, further comprising a 5′ homology arm at the 5′ end of the 3′ intron fragment.

### Embodiment 6

The RNA polynucleotide of Embodiment 3 or 4, further comprising a 3′ homology arm at the 3′ end of the 5′ intron fragment.

### Embodiment 7

The RNA polynucleotide of Embodiment 3 or 4, further comprising a 5′ homology arm at the 5′ end of the 3′ intron fragment, and a 3′ homology arm at the 3′ end of the 5′ intron fragment.

### Embodiment 8

The RNA polynucleotide of any one of Embodiments 3-7, wherein the E1 and the E2 are each independently 0 to 20 nucleotides in length, preferably 0 to 10 nucleotides in length, such as 0, 1, 2, 3, 4, 5, 6, 7, 8, 9 or 10 nucleotides.

### Embodiment 9

The RNA polynucleotide of any one of Embodiments 3-8, wherein the 5′ intron fragment and the 3′ intron fragment are obtained by segmenting a group II intron at an unpaired region into two fragments, wherein the unpaired region is preferably selected from a linear region between two adjacent domains of the group II intron and a loop region of a stem-loop structure of domain 4 of the group II intron.

### Embodiment 10

An RNA polynucleotide comprising a construct of Formula IV,

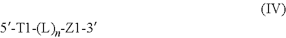

wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
each L is independently a linker sequence; and
n is an integer selected from 0 to 2.

### Embodiment 11

An RNA polynucleotide comprising an engineered translation initiation element (TI) comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341.

### Embodiment 12

The RNA polynucleotide any one of Embodiments 1-11, wherein the RNA polynucleotide is circularized via a ligation reaction.

### Embodiment 13

The RNA polynucleotide of Embodiment 12, wherein the RNA polynucleotide is circularized in the presence of a T4 ligase.

### Embodiment 14

The RNA polynucleotide of Embodiment 12, wherein the RNA polynucleotide is circularized in the absence of a T4 ligase.

### Embodiment 15

The RNA polynucleotide of any one of Embodiments 1-11, wherein the RNA polynucleotide is circularized via a splicing reaction.

### Embodiment 16

The RNA polynucleotide of Embodiment 15, wherein the RNA polynucleotide is circularized in the presence of a spliceosome.

### Embodiment 17

The RNA polynucleotide of Embodiment 15, wherein the RNA polynucleotide is circularized in the absence of a spliceosome.

### Embodiment 18

The RNA polynucleotide of any one of Embodiments 1-11, wherein the RNA polynucleotide is circularized via a self-splicing reaction.

### Embodiment 19

The RNA polynucleotide of any one of Embodiments 1-18, wherein the IRES-like polynucleotide sequence is between 6-12 residues in length.

### Embodiment 20

The RNA polynucleotide of any one of Embodiments 1-19, wherein the TI further comprises a second IRES-like polynucleotide sequence.

### Embodiment 20-1

The RNA polynucleotide of Embodiment 20, wherein the first IRES-like polynucleotide sequence and the second IRES-like polynucleotide sequence are the same.

### Embodiment 20-2

The RNA polynucleotide of Embodiment 20, wherein the first IRES-like polynucleotide sequence and the second IRES-like polynucleotide sequence are the different.

### Embodiment 21

The RNA polynucleotide of Embodiment 20, 20-1, or 20-2, wherein the second IRES-like polynucleotide sequence comprises the nucleic acid sequence of about or at least about 90%, 95%, 97%, 98%, 99% or 100% identical to SEQ ID NO: 1025-14161 or 14412-15341.

### Embodiment 22

The RNA polynucleotide of any one of Embodiments 1-21, wherein the TI further comprises a natural IRES sequence. The RNA polynucleotide of any one of Embodiments 1-21, wherein the TI further comprises an IRES sequence that is isolated or derived from a natural IRES sequence.

### Embodiment 23

The RNA polynucleotide of any one of Embodiments 1-22, wherein the IRES sequence is an RNA sequence capable of engaging a eukaryotic ribosome.

In some embodiments, the natural IRES sequence is an endogenous IRES sequence which is isolated or derived from Homo sapiens. In some embodiment, the natural IRES sequence is an endogenous IRES sequence which is isolated or derived from human tissue or human sample.

In some embodiments, the natural IRES sequence is isolated or derived from the IRES sequence of a virus, a mammal, and a Drosophila.

In some embodiments, the natural IRES sequence is isolated or derived from a picomavirus complementary DNA (cDNA), a encephalomyocarditis virus (EMCV) cDNA, poliovirus cDNA, ABPV IGRpred, AEV, ALPV IGRpred, BQCV IGRpred, BVDV1 1-385, BVDV1 29-391, CrPV 5NCR, CrPV IGR, crTMV IREScp, crTMV_IRESmp75, crTMV_IRESmp228, crTMV IREScp, crTMV IREScp, CSFV, CVB3, DCV IGR, EMCV-R, EoPV_5NTR, ERAV_245-961, ERBV_162-920, EV71_1-748, FeLV-Notch2, FMDV type C, GBV-A, GBV-B, GBV-C, gypsy_env, gypsyD5, gypsyD2, HAV HM175, HCV type 1a, HiPVJGRpred, HIV-1, HoCV1JGRpred, HRV-2, IAPVJGRpred, idefix, KBV IGRpred, LINE-1_ORF1_-1O1_to_-I, LINE-1_ORF1_-302_to_-202, LINE-1_ORF2_-138_to_-86, LINE-1_ORF1_-44_to_-1, PSIV IGR, PV type1 Mahoney, PV_type3_Leon, REV-A, RhPV 5NCR, RhPV IGR, SINV1 IGRpred, SV40 661-830, TMEV, TMV_UI_IRESmp228, TRV 5NTR, TrV IGR, or TSV IGR sequence.

In some embodiments, the natural IRES sequence of a Drosophila is an Antennapedia gene of Drosophila melanogaster.

In some embodiments, the natural IRES sequence is isolated or derived from a AML1/RUNX1, Antp-D, Antp-DE, Antp-CDE, Apaf-1, Apaf-1, AQP4, AT1R var1, AT1R_var2, AT1R_var3, AT1R_var4, BAG1_p36delta236nt, BAG1_p36, BCL2, BiP_-222_-3, C-IAP1 285-1399, cIAP1 13 13-1462, c-jun, c-myc, Cat-I_224, CCND1, DAP5, eIF4G, eIF4GI-ext, eIF4GII, eIF4GII-long, ELG1, ELH, FGF1A, FMR1, Gtx-133-141, Gtx-1-166, Gtx-1-120, Gtx-1-196, hairless, HAP4, HIF1a, hSNM1, Hsp1O1, hsp70, hsp70, Hsp90, IGF2_leader2, Kv1.4_1.2, L-myc, LamB1-335-1, LEF1, MNT 75-267, MNT 36-160, MTG8a, MYB, MYT2 997-1 152, n-MYC, NDST1, NDST2, NDST3, NDST4L, NDST4S, NRF_-653_-17, NtHSF1, ODC1, p27kip1, p53_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP 5-464, XIAP 305-466, or YAP1 sequence.

In some embodiments, the natural IRES sequence is isolated or derived from a synthetic (GAAA)16, (PPT19)4, KMI1, KMI1, KMI2, KMI2, KMIX, XI, or X2 IRES sequence.

### Embodiment 24

The RNA polynucleotide of any one of Embodiments 1-23, wherein the L comprises a 5′UTR, 3′UTR, poly-A sequence, polyA-C sequence, poly-C sequence, poly-U sequence, poly-G sequence, ribosome binding site, aptamer, riboswitch, ribozyme, small RNA binding site, translation regulation elements (e.g. a Kozak sequence), a protein binding site (e.g. PTBP1 or HUR) a non-natural nucleotide, or a non-nucleotide chemical-linker.

### Embodiment 25

The RNA polynucleotide of any one of Embodiments 1-24, wherein the L is about 3 to about 100 nucleotide residues in length.

### Embodiment 26

The RNA polynucleotide of any one of Embodiments 1-25, wherein the L comprises the nucleic acid sequence of RCC, wherein R is a guanine or an adenine.

### Embodiment 27

The RNA polynucleotide of any one of Embodiments 1-26, wherein the RNA polynucleotide is a single stranded RNA polynucleotide.

### Embodiment 28

The RNA polynucleotide of any one of Embodiments 1-27, wherein the RNA polynucleotide is a circular RNA polynucleotide.

### Embodiment 29

The RNA polynucleotide of any one of Embodiments 1-27, wherein the RNA polynucleotide is a linear RNA polynucleotide.

### Embodiment 30

The RNA polynucleotide of any one of Embodiments 1-29, wherein the RNA polynucleotide is capable of circularizing in the absence of an enzyme.

### Embodiment 31

The RNA polynucleotide of any one of Embodiments 1-30, wherein the RNA polynucleotide is an isolated RNA polynucleotide or a synthetic RNA polynucleotide.

### Embodiment 32

A polypeptide expressed by the RNA polynucleotide of any one of Embodiments 1-31.

### Embodiment 33

A DNA vector encoding the RNA polynucleotide of any one of Embodiments 1-31.

### Embodiment 34

A DNA vector suitable for synthesizing the RNA polynucleotide of any one of Embodiments 1-31.

### Embodiment 35

A cell comprising the RNA polynucleotide of any one of Embodiments 1-31, the polypeptide of Embodiment 32, or the DNA vector of Embodiment 33 or 34.

### Embodiment 36

A composition comprising the RNA polynucleotide of any one of Embodiments 1-31, the polypeptide of Embodiment 32, the DNA vector of Embodiment 33 or 34, or the cell of Embodiment 35, and a pharmaceutically acceptable carrier.

### Embodiment 37

A method of making a population of cells comprising contacting the cells of the population with the RNA polynucleotide of any one of Embodiments 1-31, the polypeptide of Embodiment 32, or the DNA vector of Embodiment 33 or 34.

### Embodiment 38

A method for generating an Internal Ribosome Entry Site (IRES)-like polynucleotide sequence, the method comprising the steps of:
(a) generating a polynucleotide query sequence consisting of X nucleic acid residues in length, wherein X is an integer greater than or equal to 3;
(b) generating X−Y+1 number of overlapping polynucleotide fragment sequences within the polynucleotide query sequence,
wherein each polynucleotide fragment sequence consists of Y nucleic acid residues in length,
wherein the first position of each polynucleotide fragment sequence is n and the last position of the same polynucleotide fragment sequence is Y+n−1, and
wherein n represents each positive integer between 1 and X−Y+1;
(c) determining an enrichment score for each polynucleotide fragment sequence of (b);
(d) determining a numerical score for the polynucleotide query sequence by summing the enrichment scores of each polynucleotide fragment sequence of (c); and
(e) identifying the polynucleotide query sequence as an engineered IRES-like polynucleotide sequence according to a reference value.

### Embodiment 38-1

The method of Embodiment 38, wherein the reference value is calculated according to the following formula: Reference Value=(23.75*X)−84.85−Z, wherein Z represents any number between 1 and 10000 and X represents the length of IRES-like polynucleotide sequence.

### Embodiment 38-2

The method of Embodiment 38-1, wherein Z represents any number between 50 and 5000.

### Embodiment 38-3

The method of Embodiment 38-1, wherein Z represents any number between 175 and 2500.

### Embodiment 38-4

The method of Embodiment 38-1, wherein Z represents any number between 150 and 200.

### Embodiment 39

The method of Embodiment 38, wherein the reference value is characteristic of the absence of a therapeutic product expression.

### Embodiment 40

The method of Embodiment 38, wherein the reference value is the mean score of all of the numerical scores of two or more IRES-like polynucleotide sequences or two or more natural IRES sequence or a combination thereof.

### Embodiment 41

The method of Embodiment 38, wherein the reference value is the average score of all of the numerical scores of two or more IRES-like polynucleotide sequences or two or more natural IRES sequence or a combination thereof.

### Embodiment 42

The method of Embodiment 38, wherein the reference value is about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% of the highest numerical score of two or more IRES-like polynucleotide sequences or two or more natural IRES sequence or a combination thereof.

### Embodiment 43

The method of Embodiment 38, wherein the reference value is about 50%, about 45%, about 40%, about 35%, about 30%, about 25%, about 20%, about 15%, or about 10% of the highest numerical score of two or more IRES-like polynucleotide sequences or two or more natural IRES sequence or a combination thereof.

### Embodiment 44

The method of Embodiment 38, wherein the reference value is at least about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% higher than the lowest numerical score of two or more IRES-like polynucleotide sequences or two or more natural IRES sequence or a combination thereof.

### Embodiment 45

The method of Embodiment 38, wherein the reference value is at least about 95%, about 90%, about 85%, about 80%, about 75%, about 70%, about 65%, about 60%, about 55%, about 50% higher than the lowest numerical score of two or more IRES-like polynucleotide sequences or two or more natural IRES sequence or a combination thereof.

### Embodiment 46

The method of Embodiment 38, wherein the reference value is greater than or equal to 0.

### Embodiment 47

The method of any one of Embodiments 38-46, wherein X is an integer selected from 3-300.

### Embodiment 48

The method of any one of Embodiments 38-47, wherein X is an integer selected from 3-100.

### Embodiment 49

The method of any one of Embodiments 38-48, wherein X is an integer selected from 5-100.

### Embodiment 50

The method of any one of Embodiments 38-49, wherein X is an integer selected from 6-100.

### Embodiment 51

The method of any one of Embodiments 38-50, wherein X is an integer greater than or equal to 5.

### Embodiment 52

The method of any one of Embodiments 38-51, wherein X is an integer greater than or equal to 6.

### Embodiment 53

The method of any one of Embodiments 38-52, wherein the polynucleotide query sequence is generated within a DNA vector suitable for synthesizing the polynucleotide query sequence.

### Embodiment 54

The method of any one of Embodiments 38-53, wherein the polynucleotide query sequence is chemically synthesized.

### Embodiment 55

The method of any one of Embodiments 38-54, wherein the overlapping polynucleotide fragment sequences within the polynucleotide query sequence are 5, 6, 7, 8, 9 or 10 nucleic acid residues in length.

### Embodiment 56

The method of any one of Embodiments 38-55, wherein the enrichment score for a polynucleotide fragment sequence is determined by:
i) generating a expression plasmid library, wherein each expression plasmid of the library comprises a different polynucleotide fragment sequence and a reporter gene;
ii) contacting a population of cells with the expression plasmid library;
iii) quantifying the expression level of the reporter gene corresponding to each expression plasmid of the expression plasmid library;
iv) dividing the total population of cells into a first population and a second population based on the protein expression levels of iii);
v) determining an enrichment score for the polynucleotide fragment sequence using the following system of equations:

$$\begin{matrix}Score = \frac{{f}_{1} - {f}_{2}}{\sqrt{( \frac{1}{{N}_{1}} + \frac{1}{{N}_{2}} ) P ( 1 - P )}} \\ P = \frac{{N}_{1} {f}_{1} - {N}_{2} {f}_{2}}{{N}_{1} + {N}_{2}}\end{matrix}$$

wherein f₁ is the frequency of the polynucleotide fragment sequence in the first population,
wherein f₂ is the frequency of the same polynucleotide fragment sequence in the second population,
wherein N₁ is the size of the first population, and
wherein N₂ is the size of the second population.

### Embodiment 57

The method of Embodiment 56, wherein the first population has a protein expression level in the top 0-10 percent of protein expression levels of the total population and wherein the second population has a protein expression level in the bottom 10-90 percent of the protein expression levels of the total population.

### Embodiment 58

The method of Embodiment 56 or 57, wherein the first population has a protein expression level in the top 50.1 percent of protein expression levels of the total population and wherein the second population has a protein expression level in the bottom 49.9 percent of the protein expression levels of the total population.

### Embodiment 59

The method of Embodiment 56 or 57, wherein the first population has a protein expression level in the top 10 percent of protein expression levels of the total population and wherein the second population has a protein expression level in the bottom 90 percent of the protein expression levels of the total population.

### Embodiment 60

The method of any one of Embodiments 38-59, wherein the polynucleotide fragment sequence is 5 nucleic acid residues in length.

### Embodiment 60-1

The method of Embodiment 60, wherein the polynucleotide fragment sequence is selected from SEQ ID NO: 1-1024 with an enrichment score as shown in Table 1.

### Embodiment 61

The method of any one of Embodiments 38-59, wherein the polynucleotide fragment sequence is 6 nucleic acid residues in length.

### Embodiment 62

The method of any one of Embodiments 38-59, wherein the polynucleotide fragment sequence is 7 nucleic acid residues in length.

### Embodiment 63

The method of any one of Embodiments 38-59, wherein the polynucleotide fragment sequence is 8 nucleic acid residues in length.

### Embodiment 64

The method of any one of Embodiments 38-59, wherein the polynucleotide fragment sequence is 9 nucleic acid residues in length.

### Embodiment 65

The method of any one of Embodiments 38-59, wherein the polynucleotide fragment sequence is 10 nucleic acid residues in length.

### Embodiment 66

The method of any one of Embodiments 38-59, wherein the polynucleotide fragment sequence is 11 nucleic acid residues in length.

### Embodiment 67

The method of any one of Embodiments 38-59, wherein the polynucleotide fragment sequence is 12 nucleic acid residues in length.

### Embodiment 68

The method of any one of Embodiments 38-59, wherein the polynucleotide fragment sequence is 13-100 nucleic acid residues in length.

### Embodiment 69

An RNA polynucleotide comprising an engineered translation initiation element (TI), wherein the TI comprises an Internal Ribosome Entry Site (IRES)-like polynucleotide sequence generated by the method of any one of Embodiments 38-68.

### Embodiment 70

A polypeptide expressed by the RNA polynucleotide of Embodiment 69.

### Embodiment 71

A DNA vector encoding the RNA polynucleotide of Embodiment 69.

### Embodiment 72

A DNA vector suitable for synthesizing the RNA polynucleotide of Embodiment 69.

### Embodiment 73

A cell comprising the RNA polynucleotide of Embodiment 69, the polypeptide of Embodiment 70, or the DNA vector of Embodiment 71 or 72.

### Embodiment 74

A composition comprising the RNA polynucleotide of Embodiment 69, the polypeptide of Embodiment 70, the DNA vector of Embodiment 71 or 72, or the cell of Embodiment 73, and a pharmaceutically acceptable carrier.

### Embodiment 75

A method of making a population of cells comprising contacting the cells of the population with the RNA polynucleotide of Embodiment 69, the polypeptide of Embodiment 70, or the DNA vector of Embodiment 71 or 72.

### Embodiment 76

A method of modulating the expression of a protein in a subject in need thereof, comprising administering to the subject a therapeutically effective amount of the polynucleotide of any one of Embodiments 1-31 and 69, the polypeptide of Embodiment 32 or 70, the DNA vector of Embodiment 33, 34, 71, or 72, or the cell of Embodiment 35 or 73.

### Embodiment 77

A method of treating or preventing a disease or disorder in a subject in need thereof, comprising administering to the subject a therapeutically effective amount of the polynucleotide of any one of Embodiments 1-31 and 69, the polypeptide of Embodiment 32 or 69, the DNA vector of Embodiment 33, 34, 71, or 72, or the cell of Embodiment 35 or 73.

### Embodiment 78

Use of the polynucleotide of any one of Embodiments 1-31 and 69, the polypeptide of Embodiment 32 or 70, the DNA vector of Embodiment 33, 34, 71, or 72, or the cell of Embodiment 35 or 73 in the manufacture of a medicament for modulating the expression of a protein or treating or preventing a disease or disorder in a subject in need thereof.

### Embodiment 79

A polynucleotide according to any one of Embodiments 1-31 and 69, a polypeptide according to Embodiment 32 or 70, a DNA vector according to Embodiment 33, 34, 71, or 72, or a cell according to claim 35 or 73 for modulating the expression of a protein or treating or preventing a disease or disorder in a subject in need thereof.

### 7. BRIEF DESCRIPTION OF THE DRAWINGS

FIG. 1 shows a schematic diagram of an IRES-like sequence generation method of the disclosure. An exemplary polynucleotide query sequence is generated (e.g., 12-mer nucleotide sequence). Overlapping polynucleotide fragment sequences within the polynucleotide query sequence are generated (e.g., pentamers 1-8). An enrichment score for each polynucleotide fragment sequence is generated (e.g., z-scores of pentamers 1-8). A numerical score for the polynucleotide query sequence is generated by summing the enrichment scores of each polynucleotide fragment sequence (e.g., sum of z-score values). The polynucleotide query sequence is identified as an IRES-like sequence when the numerical score for the polynucleotide query sequence is above a determined threshold.

FIG. 2 shows a western blot analysis of protein expression from exemplary circular RNAs having IRES-like sequences with 12 nucleotides in length (12-mer). Z-score (numerical score) and percentile ranking out of all 12-mer nucleotide sequences tested are shown.

FIG. 3 shows a schematic flow chart of an IRES-like sequence optimization method of the disclosure.

FIG. 4 shows a schematic flow chart of an IRES-like sequence optimization method of the present disclosure.

FIG. 5 is a flow chart illustrating a process of obtaining a circular RNA of the present disclosure.

FIG. 6 is a bar graph showing the luminescent signal of luciferase protein expression from exemplary circular RNAs having IRES-like sequences of the present disclosure.

FIG. 7 are images showing In Vivo Imaging System (IVIS) Spectrum of protein expression after IV injection of exemplary circular RNAs having a combination of natural IRES and IRES-like sequences of the present disclosure.

FIG. 8 are diagrams illustrating self-circularization with group I or group II intron.

FIG. 9 are gel images showing RNA self-splicing based on two splicing group II introns along with the flanking exon sequences.

FIG. 10A are gel images showing RNA splicing (upper panel) and bar graph indicating percent of RNA circularization determined by electrophoresis (lower panel).

FIG. 10B are gel images showing the results of experiments verifying the successful formation of circular RNAs by different methods.

FIG. 11 are gel images showing the results of the circularization products of the three target sequences without scars under different magnesium ion concentrations.

FIG. 12A is a flow cytometry plot showing the protein expression from cells expressing exemplary circular RNAs having a translation initiation element.

FIG. 12B shows a western blot analysis of protein expression from exemplary circular RNAs having a translation initiation element with 6 nucleotides in length (i.e., IRES-like element, 6-mer). Z-score (numerical score) and percentile ranking out of all 6-mer nucleotide sequences tested are shown.

FIG. 12C-12E shows a series of western blot analyses of protein expression from exemplary circular RNAs having a translation initiation element with 12 nucleotides in length (i.e. IRES-like element, 12-mer). Z-score (numerical score) and percentile ranking out of all 12-mer nucleotide sequences tested are shown.

FIG. 13A shows the IRES-like sequence with the highest ranked numerical score at each polynucleotide query length. The number on the left represents the total length of each nucleotide. Nucleotide length (SEQ ID NO)—Length 6 (SEQ ID NO: 1025); Length 7 (SEQ ID NO: 1225); Length 8 (SEQ ID NO: 1425); Length 9 (SEQ ID NO: 1625); Length 10 (SEQ ID NO: 1826); Length 11 (SEQ ID NO: 2026); Length 12 (SEQ ID NO: 2226); Length 13 (SEQ ID NO: 2426); Length 14 (SEQ ID NO: 2626); Length 15 (SEQ ID NO: 2827); Length 16 (SEQ ID NO: 3028); Length 17 (SEQ ID NO: 3229); Length 18 (SEQ ID NO: 3429); Length 19 (SEQ ID NO: 3629); Length 20 (SEQ ID NO: 3829); Length 21 (SEQ ID NO: 4033).

FIG. 13B shows a graph of the highest ranked numerical score for each polynucleotide query length. The y-axis shows the highest ranked numerical score (i.e. highest Z-score). The x-axis shows the length of the polynucleotide query sequence tested. The equation of the line is Numerical Score=23.74869×length of polynucleotide query sequence−84.85243.

FIG. 14A shows a graph of the predicted numerical scores for a polynucleotide query sequence length of 12, 18, 50 or 100 nucleotides. The y-axis shows the numerical score. The x-axis shows the percentile rank.

FIG. 14B shows a graph of the predicted numerical scores for a polynucleotide query sequence length of 12 nucleotides. This graph represents a zoomed image of the top percentile ranks (99.99250% to 100%). The y-axis shows the numerical score. The x-axis shows the percentile rank. For 12 mer sequences, the 99.99533% percentile corresponds to a numerical score of 165.55; the 99.99600% percentile corresponds to a numerical score of 167.43; the 99.99700% percentile corresponds to a numerical score of 171.34; the 99.99800% percentile corresponds to a numerical score of 174.88; the 99.99900% percentile corresponds to score 181.30; the 100% percentile corresponds to a numerical score 198.32.

FIG. 14C shows a graph of the predicted numerical scores for a polynucleotide query sequence length of 18 nucleotides. This graph represents a zoomed image of the top percentile ranks (99.99250% to 100%). The y-axis shows the numerical score. The x-axis shows the percentile rank. For 18 mer sequences, the 99.9950% percentile corresponds to score 210.98, the 99.9960% percentile corresponds to score 216.97, the 99.9970% percentile corresponds to score 222.21, the 99.9980% percentile corresponds to score 229.26, the 99.9990% percentile corresponds to score 240.88, the 100% percentile corresponds to score 341.65.

FIG. 14D shows a graph of the predicted numerical scores for a polynucleotide query sequence length of 50 nucleotides. This graph represents a zoomed image of the top percentile ranks (99.99250% to 100%). The y-axis shows the numerical score. The x-axis shows the percentile rank. For 50 mer sequences, the 99.9950% percentile corresponds to score 309.20, the 99.9960% percentile corresponds to score 315.56, the 99.9970% percentile corresponds to score 323.64, the 99.9980% percentile corresponds to score 335.22, the 99.9990% percentile corresponds to score 354.05, the 100% percentile corresponds to score 1101.43.

FIG. 14E shows a graph of the predicted numerical scores for a polynucleotide query sequence length of 100 nucleotides. This graph represents a zoomed image of the top percentile ranks (99.99250% to 100%). The y-axis shows the numerical score. The x-axis shows the percentile rank. For 100 mer sequences, the 99.9950% percentile corresponds to score 384.85, the 99.9960% percentile corresponds to score 393.02, the 99.9970% percentile corresponds to score 403.11, the 99.9980% percentile corresponds to score 416.68, the 99.9990% percentile corresponds to score 439.34, the 100% percentile corresponds to score 2289.57.

FIG. 15 shows a graph of the luminescent signal of luciferase protein expression from exemplary circular RNAs having IRES-like sequences of the present disclosure. The y-axis shows the luminescence value. The x-axis shows the six exemplary circular RNAs (V1, V3, V4, V5, V6 and V7), wherein V1, V3, V4, V5, V6 and V7 total length (linker sequence+IRES-like sequence), SEQ ID corresponding Z-score and SEQ ID corresponding percentile show in the table.

FIG. 16 shows a graph of group II intron's general structure. As shown, a typical group II intron can have six stem-loop structures, referred to as Domains 1-6, or D1-D6. D4 contains an open reading frame. The 6 domains are sequentially arranged and comprise multiple exon binding sequences (EBSs), such as EBS1, EBS2, and EBS3. These EBS sequences interact, such as complementarily pair, with the intron binding sequences (IBSs) in exon regions (such as IBS1, IBS2, and IBS3), to trigger self-splicing. Note that a single nucleotide, the δ nucleotide, which is located directly upstream of EBS1 in domain 1 can also pair with IBS3, and the interaction between δ and IBS3 is referred to δ-IBS3 pairing.

### 8. DETAILED DESCRIPTION

The present disclosure overcomes problems associated with current technologies by providing a novel methodology for identifying and generating synthetic Internal Ribosome Entry Site (IRES)-like sequences. The present disclosure is based, at least in part, on the development of algorithmic methods and high-throughput reporter assays that can systematically screen and quantify the IRES activity of RNA sequences that can facilitate circular RNA translation. The inventors have systematically screened pools of random polynucleotide sequences that drive protein expression and have distinguished nucleic acid sequence motifs that enhance protein expression (i.e., IRES-like sequences) from nucleic acid sequence motifs that decrease protein expression (i.e., non-IRES sequences), or nucleic acid sequence motifs that more potently enhance protein expression (i.e., IRES-like sequences) from other nucleic acid sequence motifs, using a systematic ranking methods. This is useful for identification of novel sequences with IRES activity that may function more efficiently than natural IRES sequences. This is also useful for the identification of IRES elements that are universal and species independent.

Discovery of these nucleic acid sequence motifs allows for the generation of synthetic IRES-like sequences for use in enhancing protein expression in eukaryotic cells. Increasing protein expression is desirable to improve gene expression and/or protein expression and manufacture in therapeutic applications including, but not limited to protein replacement therapy and vaccinations. Further, the use of a combination of IRES-like sequences and non-IRES sequences provides the opportunity for fine-tuned control and modulation of protein expression. The use of IRES-like sequences together with non-IRES sequences is also advantageous for the precise control of the ratio of peptide expression in the manufacture and production of multimeric proteins and/or multicistronic cassettes for therapeutic applications, such as antibody production.

Accordingly, the present disclosure also provides IRES-like sequences and polynucleotides comprising the IRES-like sequences and an expression sequence encoding a therapeutic product of interest, which may be suitable for use as a medicament or a vaccine, such as for application in gene therapy and/or genetic vaccination. In some embodiments, the present disclosure provides polynucleotides comprising the IRES-like sequences and an expression sequence encoding a therapeutic product of interest, for use in improving protein manufacture and production. In some embodiments, the polynucleotide is an RNA polynucleotide. In some embodiments, the RNA polynucleotide is a circular RNA polynucleotide. Together, the present disclosure provides improved polynucleotides comprising IRES-like sequences, which overcome the disadvantages of in the prior art by providing a cost-effective, systematic and straight forward approach for modulating protein expression.

Provided herein are non-naturally occurring RNAs having group II intron self-splicing activity which, upon self-splicing, can form circular RNAs. Circular RNAs (circRNAs) are single-stranded RNAs that are joined head to tail. As known in the art, circRNAs can be produced in vitro using chemical means or via enzymatic activities from precursor RNA, which refers to the linear RNA molecule from which a circRNA is directly generated, regardless of the methods of circularization. For example, the 5′ end and 3′ end of a linear nucleic acid can be chemically linked by the catalysis of bromine cyanide and a morpholinyl derivative, or ligated in a head-to-tail manner by the activity of a nucleic acid ligase. CircRNAs can also be produced by splicing. When a linear precursor undergoes splicing, a portion of the molecule is excised, resulting in a circRNA that has fewer total nucleotides than the precursor RNA.

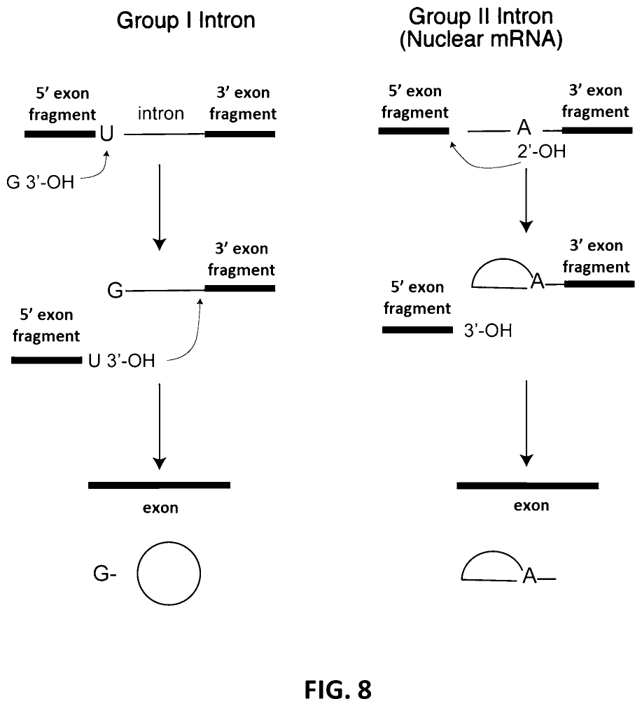

The circRNAs can also be produced by ribozyme-catalyzed RNA splicing. As used herein and understood in the art, the term “ribozyme” refers to an RNA molecule with an enzymatic activity. Some ribozymes can catalyze self-splicing independent of the spliceosome, which are referred to as “ribozymes with self-splicing activity,” “self-splicing ribozymes,” or “self-splicing introns.” Naturally occurring self-splicing ribozymes can be divided into group I and group II introns. Although the splicing products of the two categories of ribozymes are similar, the structures and splicing mechanisms of the ribozymes themselves are quite different. The group I intron has a 9-helix structure, which requires an external hydroxyl group in guanosine monophosphate (pG-OH) to trigger the reaction during catalytic splicing, and are highly dependent on the sequences of exons located at both ends of the group I intron. The group II intron relies on its own hydroxyl groups within the nucleotide sequence to trigger splicing. (See FIG. 8). This splicing mechanism is closer to the splicing reaction mediated by a spliceosome and better simulate splicing in higher organisms. The term “group I intron self-splicing activity” or “group I intron activity” refers to the self-splicing activity derived from a group I intron; and the term “group II intron self-splicing activity” or “group II intron activity” refers to the self-splicing activity derived from a group II intron. Method for preparing circRNAs based on group II intron self-splicing activities have at least the following advantages: reduction of the use of biological and chemical reagents (such as ligase and associated reagents), ease of operation, and simple design.

RNAs that are engineered ribozymes with self-splicing activity which, upon self-splicing, forms circRNAs are also referred to herein as “cRNAzymes.” In some embodiments, the cRNAzymes can have in vitro self-splicing activity. Disclosed herein are novel non-naturally occurring RNAs that have group II intron self-splicing activity and that, upon self-splicing, forms circRNAs, or “group II cRNAzymes.” Further provided herein are also vectors comprising polynucleotides encoding these group II cRNAzymes, methods of preparing the group II cRNAzymes disclosed herein by transcribing these vectors, and uses of these group II cRNAzymes in making circRNAs.

Before the present disclosure is further described, it is to be understood that the disclosure is not limited to the particular embodiments set forth herein, and it is also to be understood that the terminology used herein is for the purpose of describing particular embodiments, and is not intended to be limiting.

### 8.1. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.1%, preferably below 0.05%, and more preferably below 0.01%. Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” or “additional” may mean at least a second or more.

As used herein, the term “about” is used to indicate that a value includes the inherent variation of the value, for example, due to error for the device, the method being employed to determine the value, or the variation that exists among the study subjects. In some embodiments, “about” means that the variation is ±10%, ±9%, ±8%, ±7%, ±6%, ±5%, ±4%, ±3%, ±2%, ±1%, ±0.5%, ±0.2%, or ±0.1% of the value to which “about” refers. In some embodiments, “about” means that the variation is ±10%, ±9%, ±8%, ±7%, ±6%, ±5%, ±4%, ±3%, or ±2% of the value to which “about” refers. In some embodiments, “about” means that the variation is ±10% of the value to which “about” refers. In some embodiments, “about” means that the variation is ±5%, ±4%, ±3%, ±2, or ±1% of the value to which “about” refers. In some embodiments, “about” means that the variation is ±1%, ±0.5%, ±0.2%, or ±0.1% of the value to which “about” refers.

The terms “average” and “mean” are used interchangeably herein, unless explicitly indicated to the contrary.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. For example, in some embodiments, if a full-length polypeptide binds a given ligand, a portion of that full-length polypeptide also binds to the same ligand.

The terms “protein” and “polypeptide” are used interchangeably herein, unless explicitly indicated to the contrary.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

The term “cRNAzyme” is used herein to refer a linear ribonucleic acid (RNA) which is capable of producing a circular RNA via a self-catalyzed back-splicing reaction.

The term “cRNAzyme construct” is a linear RNA construct which has cRNAzyme activity.

The term “EBS” is used herein to refer to an exon binding sequence, which interacts with (e.g., forming a complementary pair) the intron binding sequences (IBSs) in exon regions, triggering splicing by virtue of their own hydroxyl groups within the EBS.

The term “IBS” is used herein to refer to an intron binding sequence, which interacts with (e.g., forming a complementary pair) exon binding sequences (EBSs) to locate the splicing site.

The term “EBS1” is used herein to refer to exon binding sequence 1. In some embodiments, the EBS1 comprises a nucleic acid sequence selected from the group consisting of: (a) UAGGGC; (b) UUAUGG; (c) UCAACG; and (d) UGUGGC.

The term “EBS2” is used herein to refer to exon binding sequence 2.

The term “EBS3” is used herein to refer to exon binding sequence 3.

The term “EBS1′” is used herein to refer to a modified EBS1 sequence which interacts with IBS1′. The interaction between EBS1′ and IBS1′ is similar as the interaction between EBS1 and IBS1. In some embodiments, the EBS1′ comprises a nucleic acid sequence selected from the group consisting of: (a) UAGGGC; (b) UUAUGG; (c) UCAACG; and (d) UGUGGC.

The term “EBS3′” is used herein to refer to a modified EBS3 sequence which interacts with IBS3′. The interaction between EBS3′ and IBS3′ is similar as the interaction between EBS3 and IBS3.

The term “IBS1” is used herein to refer to an intron binding sequence 1, which interacts with exon binding sequence 1 (EBS 1) to locate splicing site. In some embodiments, the IBS1 comprises a nucleic acid sequence selected from the group consisting of: (a) GCCCUG; (b) CCAUGG; (c) CGUUGA; and (d) GCCAUA.

The term “IBS1′” is used herein to refer to a region on a target sequence which has similar function of IBS1.

The term “IBS2” is used herein to refer to an intron binding sequence 2, which interacts with exon binding sequence 2 (EBS2) to locate splicing site.

The term “IBS3” is used herein to refer to an intron binding sequence 3, which interacts with exon binding sequence 3 (EBS3) to locate splicing site. In some embodiments, the IBS3 and its downstream sequence comprises a nucleic acid sequence selected from the group consisting of: (a) AGCAAA; (b) AGCAGU; (c) AGAGAA; and (d) AGCAAA.

The term “IBS3′” is used herein to refer to a region on a target sequence which has similar function of IBS3.

The term “δ” (delta) is used herein to refer to a region in domain 1 of a group II intron which is the single nucleotide directly upstream of EBS1. δ pairs with IBS3 and the interaction between δ and IBS3 is called δ-IBS3 pairing. In some embodiments, the 6 sequence and its upstream comprises a nucleic acid sequence selected from the group consisting of: (a) UGUGCU; (b) AAUGCU; (c) UGCUCU; and (d) UGUGCU.

The term “δ″” (delta″) is used herein to refer to a region in domain I of a group II intron which is the single nucleotide directly upstream of EBS1′. δ″ pairs with IBS3′ and the interaction between δ″ and IBS3′ is called δ″-IBS3′ pairing.

As used herein, the term “group II intron” and “Group II intron” are used herein interchangeably to refer to RNA molecules which are encoded by the group II introns, share a similar secondary and tertiary structure. Group II intron RNA molecules typically have six domains. Group II intron mainly comprises 6 stem-loop structures, called domains 1 to 6 (D1 to D6), and the 6 domains are arranged in sequence, comprising multiple exon binding sequences (EBSs), such as EBS1, EBS2, and EBS3.

A group II intron may have a modification of one or more nucleotides relative to its wild-type form, and the modification is selected from one or more of a deletion, a substitution, and an addition. In some embodiments, the modification comprises a modification of one or more EBS sequences of the group II intron, wherein the EBS sequences are complementarily paired with one or more regions of a corresponding length in a target sequence on at least 60% of the nucleotide positions respectively. In some embodiments, the modification is a modification of the two EBS sequences of the group II intron, such as EBS1 and EBS3, wherein the EBS sequences are complementarily paired with two regions of a corresponding length in a target sequence on at least 60% of the nucleotide positions respectively; preferably, the two regions are located at both ends of the target sequence, respectively. In some embodiments, the modification is a modification of the two EBS sequences of the group II intron, such as EBS1′ and EBS3′, wherein the EBS sequences are complementarily paired with two regions of a corresponding length in a target sequence on at least 60% of the nucleotide positions respectively; preferably, the two regions are located at both ends of the target sequence, respectively. In some embodiments, the modification is a modification of EBS1 and/or δ sequence of the group II intron, or a modification of EBS1′ and/or δ sequence, wherein the EBS1 and/or δ sequence is complementarily paired with a region of a corresponding length in a target sequence on at least 60% of the nucleotide, optionally the modification is a modification of EBS1 and/or δ sequence and its upstream sequence, wherein the EBS1 and/or δ sequence and its upstream is complementarily paired with a region of a corresponding length in a target sequence on at least 60% of the nucleotide. In some embodiments, the modification is a modification of EBS1 and/or δ sequence of the group II intron, or a modification of EBS1′ and/or δ″ sequence, wherein the EBS1 and/or δ sequence is complementarily paired with a region of a corresponding length in a target sequence on at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotide, optionally the modification is a modification of EBS1 and/or δ sequence and its upstream sequence, wherein the EBS1 and/or δ sequence and its upstream sequence is complementarily paired with a region of a corresponding length in a target sequence on at least 60% of the nucleotide. In some embodiments, the region of a corresponding length in a target sequence is IBS3, IBS3′, IBS3 with downstream sequence, or IBS3′ with downstream sequence. In some embodiments, the modification comprises a deletion of part or all of domain 4, such as a deletion of an intron-encoded protein (IEP) sequence in domain 4, preferably a deletion of all of domain 4. In some embodiments, the modification comprises a deletion of an open reading frame (ORF).

The term “domain 1” or “D1” is used herein to refer to a stem-loop structure of domain 1 of a Group II intron. The term “domain 2” or “D2” is used herein to refer to a stem-loop structure of domain 2 of a Group II intron. The term “domain 3” or “D3” is used herein to refer to a stem-loop structure of domain 3 of a Group II intron. The term “domain 4” or “D4” is used herein to refer to a stem-loop structure of domain 4 of a Group II intron. The term “domain 5” or “D5” is used herein to refer to a stem-loop structure of domain 5 of a Group II intron. The term “domain 6” or “D6” is used herein to refer to a stem-loop structure of domain 6 of a Group II intron. Stem-loop structure is a type of an RNA secondary structure, which can be determined by any suitable polynucleotide folding algorithm. Some programs are based on the calculation of the minimum Gibbs free energy. An exemplary algorithm is mFold (Zuker and Stiegler, Nucleic Acids Res. 9, 133-148 (1981)). Other exemplary folding algorithms are known in the art, for example, as described in A R Gruber et al., Cell 106, 23-24 (2008), P A Carr and G M Church, Nature Biotechnology 27, 1151-62 (2009)), PCT Application No. WO 2014093709, which is incorporated herein by reference.

The term “5′ intron fragment” and “3′ intron fragment” are used herein to refer the intron fragments obtained by segmenting a group II intron at a loop region of a stem-loop structure of domain 1, 2, 3, 4, 5, or 6.

The terms “nucleic acid sequence”, “polynucleotide”, and “oligonucleotide” are used interchangeably herein, unless specified otherwise or the context indicates to the contrary, and refer to a polymer or oligomer of pyrimidine and/or purine bases, such as cytosine, thymine and uracil, adenine, and guanine, respectively (see Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. A nucleic acid or nucleic acid sequence may comprise other kinds of nucleic acid structures such as, for instance, a DNA/RNA helix, peptide nucleic acid (PNA), morpholino nucleic acid (see, e.g., Braasch and Corey, Biochemistry, 4/(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, Am. Chem. Soc., 122: 8595-8602 (2000)), and/or a ribozyme. The terms “nucleic acid”, “nucleic acid sequence”, “polynucleotide”, and “oligonucleotide” may also encompass a chain comprising non-natural nucleotides, modified nucleotides, and/or non-nucleotide building blocks that can exhibit the same function as natural nucleotides (e.g., “nucleotide analogs”). The term “DNA sequence” is used herein to refer to a nucleic acid comprising a series of DNA bases.

As is understood in the art, a nucleic acid strand is inherently directional, as the carbon atoms in the sugar ring are numbered from 1′ to 5′ and the “5′-end” has a free hydroxyl (or phosphate) on a 5′ carbon and the “3′ prime end” has a free hydroxyl (or phosphate) on a 3′ carbon. As used herein and understood in the art, a nucleic acid having certain sequence elements “from 5′ to 3′” means that these sequence elements are arranged linearly from the 5′ end to the 3′ end of the nucleic acid.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specific are not limited to, adenine (A) and thymine (T); adenine (A) and uracil (U); cytosine (C) and guanine (G); and 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, the term “nucleobase” means an unmodified nucleobase or a modified nucleobase. As used herein “an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methyleytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, the term “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are present between those that are linked).

The terms “polypeptide” and “protein” are used interchangeably herein, unless specified otherwise or the context indicates to the contrary, and refer to a polymeric form of amino acids comprising at least two or more contiguous amino acids chemically or biochemically modified or derivatized amino acids. The term “peptide” as used herein refers to a class of short polypeptides. The term peptide may refer to a polymer of amino acids (natural or non-naturally occurring) having a length of up to about 100 amino acids. For example, peptides may be about 1 to about 10, about 10 to about 25, about 25 to about 50, about 50 to about 75, about 75 to about 100 amino acid residues in length. In some embodiments, the peptides may be about 100, about 200, about 300, about 400, about 500, about 600, about 700, about 800, about 900, about 1000, about 1250, about 1500, about 1750, about 2000, about 2250, about 2500, about 2750, about 3000, about 3250, about 3500, about 3750, about 4000, about 4250, about 4500, about 4750, are about 5000 amino acid residues in length.

Nomenclature for nucleotides, nucleic acids, nucleosides, and amino acids used herein is consistent with International Union of Pure and Applied Chemistry (IUPAC) standards (see, e.g., bioinformatics.org/smsylupac.html).

When referring to a nucleic acid sequence or protein sequence, the term “identity” is used to denote similarity between two sequences. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection. Another algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

The terms “internal ribosome entry site,” “internal ribosome entry site sequence,” “IRES” and “IRES sequence region” are used interchangeably herein and refer to cis elements of viral or human cellular RNAs (e.g., messenger RNA (mRNA) and/or circRNAs) that bypass the steps of canonical eukaryotic cap-dependent translation initiation. The canonical cap-dependent mechanism used by the vast majority of eukaryotic mRNAs requires an m⁷G cap at the 5′ end of the mRNA, initiator Met-tRNAmet, more than a dozen initiation factor proteins, directional scanning, and GTP hydrolysis to place a translationally competent ribosome at the start codon. IRESs typically are comprised of a long and highly structured 5-UTR which mediates the translation initiation complex binding and catalyzes the formation of a functional ribosome.

The term “IRES-like sequence” or “Internal Ribosome Entry Site-like sequence” refer to synthetic nucleotide sequences that display a function of a natural IRES. In some embodiments, the IRES-like sequence can recruit ribosomal components to mediate cap-independent translation.

The terms “coding sequence,” “coding sequence region,” “coding region,” and “CDS” when referring to nucleic acid sequences may be used interchangeably herein to refer to the portion of a DNA or RNA sequence, for example, that is or may be translated to protein. The terms “reading frame,” “open reading frame,” and “ORF,” may be used interchangeably herein to refer to a nucleotide sequence that begins with an initiation codon (e.g., ATG) and, in some embodiments, ends with a termination codon (e.g., TAA, TAG, or TGA). Open reading frames may contain introns and exons, and as such, all CDSs are ORFs, but not all ORF are CDSs.

The term “X-mer” refers to in a polynucleotide sequence (e.g., an IRES-like sequence) with an “X” number of nucleic acid residues/nucleotides (wherein “X” is an integer). For example, “10-mer” indicates a polynucleotide sequence of the present disclosure having 10 nucleic acid residues/nucleotides.

The terms “complementary” and “complementarity” refers to the relationship between two nucleic acid sequences or nucleic acid monomers having the capacity to form hydrogen bond(s) with one another by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., about 50%, about 60%, about 70%, about 80%, about 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., at least 65%, at least 70%, at least 75%, at least 80%, at least 85%, at least 90%, at least 95%, at least 97%, at least 98%, at least 99%, or 100%) over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleic acid sequences hybridize under at least moderate, or, in some embodiments high, stringency conditions. Exemplary moderate stringency conditions include overnight incubation at 37° C. in a solution comprising 20% formamide, 5% SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1*SSC at about 37-50° C., or substantially similar conditions, e.g., the moderately stringent conditions described in Sambrook, J., Molecular Cloning: A Laboratory Manual, Cold Spring Harbor Laboratory Press; 4th edition (Jun. 15, 2012). High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.10% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 pg/ml), 0.1% SDS, and 10% dextran sulfate at 42° C., with washes at (i) 42° C. in 0.2*SSC, (ii) 55° C. in 50% formamide, and (iii) 55° C. in 0.1*SSC (optionally in combination with EDTA). Additional details and an explanation of stringency of hybridization reactions are provided in, e.g., Sambrook, supra, and Ausubel et al., eds., Short Protocols in Molecular Biology, 5th ed., John Wiley & Sons, Inc., Hoboken, N.J. (2002).

The term “animal” refers to human and non-human animals, including non-human primates (e.g., bonobos, chimpanzees, gorillas, monkeys), and other animals, such as birds, pigs, horses, dogs, cats, rabbits, mice, rats, cows, sheep, goats, and deer.

The term “hybridization” or “hybridized” when referring to nucleic acid sequences is the association formed between and/or among sequences having complementarity.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need to be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to an RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and a functional effector element) are connected in such a way as to permit transcription of the nucleic acid molecule.

The term “homology” refers to the percent of identity between the nucleic acid residues of two polynucleotides or the amino acid residues of two polypeptides. The correspondence between one sequence and another can be determined by techniques known in the art. For example, homology can be determined by a direct comparison of the sequence information between two polypeptides by aligning the sequence information and using readily available computer programs. Two polynucleotide (e.g., DNA) or two polypeptide sequences are “substantially homologous” to each other when at least about 80%, preferably at least about 90%, and most preferably at least about 95% of the nucleotides, or amino acids, respectively match over a defined length of the molecules, as determined using the methods above.

“Treating” or “treatment of a disease or condition” refers to executing a protocol or treatment plan, which may include administering one or more drugs or active agents to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. In addition, “treating” or “treatment” does not require complete alleviation of signs or symptoms, and does not require a cure.

The term “therapeutic benefit” or “therapeutically effective” as used throughout this disclosure refers to anything that promotes or enhances the well-being of the subject with respect to the medical treatment of this condition. This includes, but is not limited to, a reduction in the frequency, severity, or rate of progression of the signs or symptoms of a disease. For example, treatment of cancer may involve, for example, a reduction in the size of a tumor, a reduction in the invasiveness of a tumor, reduction in the growth rate of the cancer, or a reduction in the rate of metastasis or recurrence. Treatment of cancer may also refer to prolonging survival of a subject with cancer.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous biocompatible solvents (e.g., saline solutions, phosphate buffered saline, parenteral vehicles, such as sodium chloride, Ringer's dextrose, etc.), antioxidants, preservatives (e.g., antibacterial or antifungal agents, anti-oxidants, chelating agents, and inert gases), isotonic agents, such like materials and combinations thereof, as would be known to one of ordinary skill in the art. The pH and exact concentration of the various components in a pharmaceutical composition are adjusted according to well-known parameters.

The term “bulged adenosine” (also known as bulged A) as used herein refer to residue within the intron sequence as the initiating nucleophile. The conventional group II-type bulged adenosine is located on domain 6 (D6, or DVI) of a group II intron. In some group II introns, the bulged adenosine is 7 or 8 nucleotides away from the 3′ splicing site. The bulged adenosine is normally conserved and plays a central role in the splicing process. During this process, The 2′ hydroxyl of the bulged adenosine attacks the 5′ splice site, followed by nucleophilic attack on the 3′ splice site by the 3′ OH of the upstream exon. This results in a branched intron lariat connected by a 2′ phosphodiester linkage at the bulged adenosine (Van der Veen et al., The EMBO Journal, 6(12):3827-3831 (1987); Jacquier et al., Journal of molecular biology, 219(3): 415-428 (1991); Daniels et al., Journal of molecular biology, 256(1):31-49 (1996)).

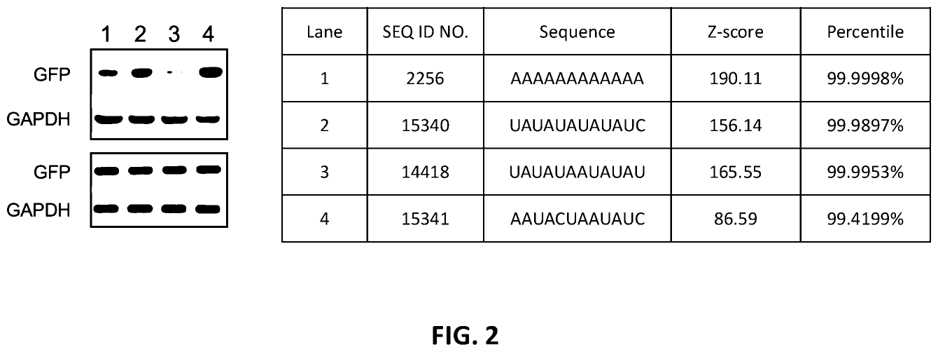

The term “atypical bulged adenosine” (also known as atypical bulged A) as used herein refer to a region found within D6 of a group JIB intron C.te.I1 (Cte), found in the in the human pathogen Clostridium tetani. D6 of Cte does not have a clearly bulged adenosine. Instead, it has a looped region, see FIGS. 2A-2E, which acts as the nucleophile for the first step of splicing (the branching pathway). (McNeil et al., RNA, 20(6):855-866 (2014))

The term “catalytic triad” as used herein refer to a highly conserved region (AGC) which forms base triples with other nucleotides to form a triple helix known as the “catalytic triplex”. This catalytic triplex forms the binding pocket for the two active site magnesium ions. (Chan et al. Nature Comm. 9.1 (2018): 1-10.)

The terms “scar,” as used herein, refers to the non-target sequence region in the circRNA splicing product. The term “scarless splicing” as used herein refers to the self-splicing of the cRNAzymes which produce circRNAs that do not contain additional sequence elements beyond the target sequence. As such, a “scarless” circRNA contains no scar, meaning that it solely consists of the target sequence. The term “near-scarless splicing” as used herein refers to the self-splicing of the cRNAzymes which produce circRNAs that include no more than 20 nucleotides besides the target sequence. A “near-scarless circRNA” is a circRNA resulting from “near-scarless” self-splicing of a cRNAzyme, which include no more than 20 nucleotides besides the target sequence.

The term “in vitro transcription,” or “IVT,” refers to versatile method to produce RNA in vitro that uses an RNA polymerase, ribonucleotides, and appropriate buffer conditions to synthesize RNA from a DNA template.

The term “resulting target sequence,” as used herein, refers to the target sequence as it is formed in the circRNA upon self-splicing of the RNAs (or cRNAzymes) provided herein.

### 8.2. Cap-Independent Translation Initiation Elements

In some embodiments, provided herein is an RNA polynucleotide comprising an engineered translation initiation element (TI) comprising an internal ribosome entry site (IRES)-like polynucleotide sequence. In some embodiments, the IRES-like polynucleotide sequence can mediate cap-independent translation initiation.

Translation initiation of mRNAs in eukaryotic cells is a complex process that involves the concerted interaction of numerous factors (Pain (1996) Eur. J. Biochem. 236, 747-771). For most mRNAs, the first step is the recruitment of ribosomal 40S subunits onto the mRNA at or near the capped 5′ end. Association of 40S to mRNA is greatly facilitated by the cap-binding protein complex eIF4F. Factor eIF4F is composed of three subunits: the RNA helicase eIF4A, the cap-binding protein eIF4E, and the multi-adaptor protein eIF4G which acts as a scaffold for the proteins in the complex and has binding sites for eIF4E, eIF4a, eIF3, and poly(A) binding protein.

Circular RNA is a type of single-stranded, covalently closed-loop RNA, and does not contain the 5′ cap that is commonly known to be required for cap-dependent translation. Thus, circular RNA translation utilizes alternate mechanisms to initiate cap-independent translation, such as the use of an internal ribosome entry site (IRES) sequence that is recognized by ribosomes. See, e.g., Wesselhoeft, R. A. et al., Nat. Commun. 9, 2629 (2018) and Chinese Patent Application No. 202110594352.4.

### 8.2.1. Natural Internal Ribosome Entry Site (IRES) Sequences

In some embodiments, the RNA polynucleotide, the precursor RNA, and the circular RNA provided herein comprises a natural internal ribosome entry site (IRES) sequence. In some embodiments, the IRES sequence is an RNA sequence capable of engaging a ribosome (e.g., eukaryotic ribosome). An IRES sequence permits the translation of one or more open reading frames from a circular RNA (e.g., open reading frames that form the expression sequence). The IRES attracts a ribosomal (e.g., eukaryotic ribosomal) translation initiation complex and promotes translation initiation.

A multitude of natural IRES sequences are available and include sequences derived from or isolated from a wide variety of viruses, such as from leader sequences of piconaviruses such as the encephalomyocarditis virus (EMCV) UTR, the polio leader sequence, the hepatitis A virus leader sequence, the hepatitis C virus IRES, human rhinovirus type 2 IRES, an IRES element from the foot and mouth disease virus, a giardiavirus IRES, and the like.

In some embodiments, the natural IRES sequence is isolated or derived from an IRES sequence of Taura syndrome virus, Tiiatoma virus, Theiler's encephalomyelitis virus, Simian Virus 40, Solenopsis invicta virus 1, Rhopalosiphum padi virus, Reticuloendotheliosis virus, Human poliovirus 1, Plautia stall intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis A Virus HA 16, Hepatitis GB virus, Foot and mouth disease virus, Human enterovirus 71, Equine rhinitis virus, Ectrapis obliqua picoma-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, tobacco etch virus, turnip crinkle virus, EMCV-A, EMCV-B, EMCV-Bf, EMCV-Cf, EMCV pEC9, Picobimavirus, HCV QC64, Human Cosavirus E/D, Human Cosavirus F, Human Cosavirus JMY, Rhinovirus NAT001, HRV14, HRV89, HRVC-02, HRV-A21, Salivirus A SHI, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistravirus, Hubei Picoma-like Virus, CRPV, Salivirus A BN5, Salivirus A BN2, Salivirus A 02394, Salivirus A GUT, Salivirus A CH, Salivirus A SZ1, Salivirus FHB, Coxsackievirus (e.g., CVA3, CVA12, CVB1, CVB3, CVB5), Echovirus 7, Enterovirus A71, and/or EV24.

In some embodiments, a natural IRES sequence is isolated or derived from a eukaryotic IRES element selected from Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIFI alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, and S. cerevisiae YAP1.

In some embodiments, a natural IRES sequence is an endogenous IRES sequence, which is derived from or isolated from Homo sapiens. In some embodiments, a natural IRES sequence is an endogenous IRES sequence, which is derived from or isolated from human tissue or human sample.

In some embodiments, an IRES sequence is isolated or derived from a cellular IRES element selected from AML1/RUNX1, Antp-D, Antp-DE, Antp-CDE, AT1R var1, AT1R_var2, AT1R_var3, AT1R_var4, BAG1_p36delta236nt, BAG1_p36, BiP_-222_-3, C-IAP1 285-1399, c IAP1 13 13-1462, c-jun, Cat-1_224, CCND1, eIF4GI-ext, eIF4GII, eIF4GII-long, FGF1A, FMR1, Gtx-133-141, Gtx-1-166, Gtx-1-120, Gtx-1-196, HAP4, HIF1a, hSNM1, Hsp1O1, hsp70, hsp70, Hsp90, IGF2_leader2, L-myc, MNT 75-267, MNT 36-160, MTG8a, MYB, MYT2 997-1 152, NRF_-653_-17, NtHSF1, ODC1, p27kip1, p53_128-269, PDGF2/c-sis, PITSLRE_p58, Rbm3,reaper, Scamper, TFIID, TIF4631, Ubx_1-966, Ubx_373-961, UNR, Ure2, XIAP 5-464, XIAP 305-466,YAP1, (GAAA)16, (PPT19)4 and XI.

In some embodiments, an IRES sequence is isolated or derived from a viral IRES element selected from ABPV IGRpred, AEV, ALPV IGRpred, BQCV IGRpred, BVDV1 1-385, BVDV1 29-391, CrPV 5NCR, CrPV IGR, crTMV_IRESmp228, CSFV, DCV IGR, EoPV_5NTR, ERBV_162-920, EV71_l-748, FMDV type C, GBV-A, GBV-C, HAV HM175, HiPVJGRpred, HIV-1, HoCV1JGRpred, IAPVJGRpred, idefix, KBV IGRpred, PSIV IGR, PV type1 Mahoney, PV_type3_Leon, REV-A, RhPV 5NCR, RhPV IGR, SINV1 IGRpred, SV40 661-830, TMEV, TMV_UI_IRESmp228, TRV 5NTR, TrV IGR, TSV, and IGR.

In some embodiments, the IRES sequence of the present disclosure comprises a sequence isolated or derived from a natural IRES sequence. Exemplary natural IRES sequences include but are not limited to those listed in Tables 98-100. In some embodiments, the IRES sequence comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the natural IRES sequences of Tables 98-100 or a functional portion thereof.

### 8.2.2. IRES-Like Sequences

In some embodiments, the RNA polynucleotide, the precursor RNA, and the circular RNA provided herein comprise an IRES-like sequence. As used herein, the term “IRES-like sequence” refers to a synthetic or artificial IRES sequence that has the function of a natural IRES sequence. In some embodiments, the IRES-like sequence is an RNA sequence capable of recruiting a ribosome (e.g., eukaryotic ribosome). In some embodiments, the IRES-like sequence is an RNA sequence capable of mediating cap-independent translation initiation. An IRES-like sequence may be identified by any one of the methods disclosed herein.

In some embodiments the IRES-like sequence is greater than or equal to 3 nucleic acid residues in length. In some embodiments, the IRES-like sequence is 3-300 nucleic acid residues in length. In some embodiments, the IRES-like sequence is 3-200 nucleic acid residues in length. In some embodiments, the IRES-like sequence is 4-200 nucleic acid residues in length. In some embodiments, the IRES-like sequence is 5-200 nucleic acid residues in length. In some embodiments, the IRES-like sequence is 6-200 nucleic acid residues in length. In some embodiments, the IRES-like sequence is 7-200 nucleic acid residues in length. In some embodiments, the IRES-like sequence is 3-100 nucleic acid residues in length. In some embodiments, the IRES-like sequence is 4-100 nucleic acid residues in length. In some embodiments, the IRES-like sequence is 5-100 nucleic acid residues in length. In some embodiments, the IRES-like sequence is 6-100 nucleic acid residues in length. In some embodiments, the IRES-like sequence is 7-100 nucleic acid residues in length. In some embodiments, the IRES-like sequence is 3-50 nucleic acid residues in length. In some embodiments, the IRES-like sequence is 4-50 nucleic acid residues in length. In some embodiments, the IRES-like sequence is 5-50 nucleic acid residues in length. In some embodiments, the IRES-like sequence is 6-50 nucleic acid residues in length. In some embodiments, the IRES-like sequence is 7-50 nucleic acid residues in length. In some embodiments, the IRES-like sequence is 3-40 nucleic acid residues in length. In some embodiments, the IRES-like sequence is 4-40 nucleic acid residues in length. In some embodiments, the IRES-like sequence is 5-40 nucleic acid residues in length. In some embodiments, the IRES-like sequence is 6-40 nucleic acid residues in length. In some embodiments, the IRES-like sequence is 7-40 nucleic acid residues in length. In some embodiments, the IRES-like sequence is 3-30 nucleic acid residues in length. In some embodiments, the IRES-like sequence is 4-30 nucleic acid residues in length. In some embodiments, the IRES-like sequence is 5-30 nucleic acid residues in length. In some embodiments, the IRES-like sequence is 6-30 nucleic acid residues in length. In some embodiments, the IRES-like sequence is 7-30 nucleic acid residues in length. In some embodiments, the IRES-like sequence is 3-20 nucleic acid residues in length. In some embodiments, the IRES-like sequence is 4-20 nucleic acid residues in length. In some embodiments, the IRES-like sequence is 5-20 nucleic acid residues in length. In some embodiments, the IRES-like sequence is 6-20 nucleic acid residues in length. In some embodiments, the IRES-like sequence is 7-20 nucleic acid residues in length. In some embodiments, IRES-like sequence is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length. In some embodiments, the IRES-like sequence is 5 nucleic acid residues in length. In some embodiments, the IRES-like sequence is 6 nucleic acid residues in length. In some embodiments, the IRES-like sequence is 7 nucleic acid residues in length. In some embodiments, the IRES-like sequence is 8 nucleic acid residues in length. In some embodiments, the IRES-like sequence is 9 nucleic acid residues in length. In some embodiments, the IRES-like sequence is 10 nucleic acid residues in length. In some embodiments, the IRES-like sequence is 11 nucleic acid residues in length. In some embodiments, the IRES-like sequence is 12 nucleic acid residues in length.

Exemplary IRES-like sequences of the present disclosure include but are not limited to those listed in Tables 2-96, 110 and 112. In some embodiments, the IRES-like sequence comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the IRES-like sequences of Tables 2-96, 110 and 112 or a functional portion thereof.

In some embodiments, the IRES-like sequence described herein or a portion thereof may be combined with a natural IRES sequence described herein or a portion thereof, to form further IRES-like sequences. In some embodiments, a complete natural IRES sequence is combined with a complete IRES-like sequence. In some embodiments, a portion of a natural IRES sequence is combined with a complete IRES-like sequence. In some embodiments, a complete natural IRES sequence is combined with a portion of an IRES-like sequence. In some embodiments, a portion of a natural IRES sequence is combined with a portion of an IRES-like sequence. Exemplary sequences comprising a portion of a natural IRES sequence combined with a portion of a IRES-like sequence include but are not limited to those listed in Table 97.

### 8.2.2.1. Methods of Generating IRES-Like Sequences

(a) The disclosure provides a method for generating an IRES-like polynucleotide sequence, the method comprising the steps of: generating a polynucleotide query sequence consisting of X nucleic acid residues in length (i.e., X-mer), wherein X is an integer greater than or equal to 3;
(b) generating X−Y+1 number of overlapping polynucleotide fragment sequences within the polynucleotide query sequence,
wherein each polynucleotide fragment sequence consists of Y nucleic acid residues in length,
wherein the first position of each polynucleotide fragment sequence is n and the last position of the same polynucleotide fragment sequence is Y+n−1, and
wherein n represents each positive integer between 1 and X−Y+1;
(c) determining an enrichment score for each polynucleotide fragment sequence of (b);
(d) determining a numerical score for the polynucleotide query sequence by summing the enrichment scores of each polynucleotide fragment sequence of (c); and
(e) identifying the polynucleotide query sequence as an engineered IRES-like polynucleotide sequence according to a reference value.

In some embodiments, the reference value is determined as described herein, for example, in 8.2.2.1.a. Generating Polynucleotide Query Sequences 8.2.2.1.b. Generating Overlapping Polynucleotide Subsequences within the Query Sequences.

In some embodiments, the reference value is characteristic of the absence of a therapeutic product expression.

In some embodiments, the reference value is the average score of all of the numerical scores of two or more IRES-like polynucleotide sequences or two or more natural IRES sequence or a combination thereof.

In some embodiments, the reference value is about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% of the highest numerical score of two or more IRES-like polynucleotide sequences or two or more natural IRES sequence or a combination thereof.

In some embodiments, the reference value is about 50%, about 45%, about 40%, about 35%, about 30%, about 25%, about 20%, about 15%, or about 10% of the highest numerical score of two or more IRES-like polynucleotide sequences or two or more natural IRES sequence or a combination thereof.

In some embodiments, the reference value is at least about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% higher than the lowest numerical score of two or more IRES-like polynucleotide sequences or two or more natural IRES sequence or a combination thereof.

In some embodiments, the reference value is at least about 95%, about 90%, about 85%, about 80%, about 75%, about 70%, about 65%, about 60%, about 55%, about 50% higher than the lowest numerical score of two or more IRES-like polynucleotide sequences or two or more natural IRES sequence or a combination thereof.

In some embodiments, the reference value is greater than or equal to 0.

In some embodiments, the numerical score for the polynucleotide query sequence is calculated using a system of equations, wherein the numerical score for the polynucleotide query sequence is greater or equal to 0.

The number of nucleic acid residues in the polynucleotide query sequence (i.e., X) is described in detail herein, e.g., 8.2.2.1.a. Generating Polynucleotide Query Sequences and 8.2.2.1.b. Generating Overlapping Polynucleotide Subsequences within the Query Sequences.

In some embodiments, X is an integer selected from 3-300. In some embodiments, X is an integer selected from 3-100. In some embodiments, X is an integer selected from 5-100. In some embodiments, X is an integer selected from 6-100. In some embodiments, X is an integer greater than or equal to 5. In some embodiments, X is an integer greater than or equal to 6.

In some embodiments, the polynucleotide query sequence is generated within a DNA vector suitable for synthesizing the polynucleotide query sequence. In some embodiments, the polynucleotide query sequence is chemically synthesized.

In some embodiments, the enrichment score for a polynucleotide fragment sequence is determined by: i) generating a expression plasmid library, wherein each expression plasmid of the library comprises a different polynucleotide fragment sequence and a reporter gene, ii) contacting a population of cells with the expression plasmid library; iii) quantifying the expression level of the reporter gene corresponding to each expression plasmid of the expression plasmid library; iv) dividing the total population of cells into a first population and a second population based on the protein expression levels of iii); v) determining an enrichment score for the polynucleotide fragment sequence.

The enrichment score for a polynucleotide fragment sequence can be determined according to the methods described herein, e.g., 8.2.2.1.c. Determining an Enrichment Score.

The numerical score for the polynucleotide query sequence can be determined according to the methods described herein, e.g., 8.2.2.1.d. Determining a Numerical Score of a Polynucleotide Sequence and Identifying an IRES-like Sequence.

### 8.2.2.1.a. Generating Polynucleotide Query Sequences

A random polynucleotide query sequence library may be generated by any suitable method known in the art or described herein. In some embodiments, the random polynucleotide query sequence (e.g., RNA polynucleotide) is generated using any DNA vector suitable for synthesizing the polynucleotide query sequence. In some embodiments, the random polynucleotide query sequence (e.g., RNA polynucleotide) is generated by chemical synthesis, error-prone PCR, or reverse-transcription PCR of transcriptome. For example, to produce a random 10-mer sequence library, a foldback primer (ATTCCGTCTCAAGTAANNNNNNNNNNATCATGGAGACGCACTGTTTTTTTCAGTGCGTCTCCATGA (SEQ ID NO: 15342)) may be generated using Klenow fragment (NEB), and the resulting DNA may be digested with BsmBI and ligated into BsmBI digested pcircGFP-BsmBI. The ligation product may be transformed into ElectroMax DH-5a (Invitrogen), to produce E. coli clones to achieve ˜2-fold coverage of all possible DNA decamers (total 2 million clones).

In some embodiments the random polynucleotide query sequence is greater than or equal to 3 residues in length. In some embodiments, the random polynucleotide query sequence is 3-300 nucleic acid residues in length. In some embodiments, the random polynucleotide query sequence is 3-200 nucleic acid residues in length. In some embodiments, the random polynucleotide query sequence is 3-100 nucleic acid residues in length. In some embodiments, the random polynucleotide query sequence is 5-100 nucleic acid residues in length. In some embodiments, the random polynucleotide query sequence is 6-100 nucleic acid residues in length. In some embodiments, the random polynucleotide query sequence is 7-100 nucleic acid residues in length. In some embodiments, the random polynucleotide query sequence is 7-90 nucleic acid residues in length. In some embodiments, the random polynucleotide query sequence is 7-80 nucleic acid residues in length. In some embodiments, the random polynucleotide query sequence is 7-70 nucleic acid residues in length. In some embodiments, the random polynucleotide query sequence is 7-60 nucleic acid residues in length. In some embodiments, the random polynucleotide query sequence is 7-50 nucleic acid residues in length. In some embodiments, the random polynucleotide query sequence is 7-40 nucleic acid residues in length. In some embodiments, the random polynucleotide query sequence is 7-30 nucleic acid residues in length. In some embodiments, the random polynucleotide query sequence is 7-20 nucleic acid residues in length. In some embodiments, the random polynucleotide query sequence is 7-15 nucleic acid residues in length. In some embodiments, the random polynucleotide query sequence is 6-90 nucleic acid residues in length. In some embodiments, the random polynucleotide query sequence is 6-80 nucleic acid residues in length. In some embodiments, the random polynucleotide query sequence is 6-70 nucleic acid residues in length. In some embodiments, the random polynucleotide query sequence is 6-60 nucleic acid residues in length. In some embodiments, the random polynucleotide query sequence is 6-50 nucleic acid residues in length. In some embodiments, the random polynucleotide query sequence is 6-40 nucleic acid residues in length. In some embodiments, the random polynucleotide query sequence is 6-30 nucleic acid residues in length. In some embodiments, the random polynucleotide query sequence is 6-20 nucleic acid residues in length. In some embodiments, the random polynucleotide query sequence is 6-15 nucleic acid residues in length.

In some embodiments, the random polynucleotide query sequence is at least 5 nucleic acid residues in length. In some embodiments, the random polynucleotide query sequence is at least 6 nucleic acid residues in length. In some embodiments, the random polynucleotide query sequence is at least 7 nucleic acid residues in length. In some embodiments, the random polynucleotide query sequence is at least 8 nucleic acid residues in length. In some embodiments, the random polynucleotide query sequence is at least 9 nucleic acid residues in length. In some embodiments, the random polynucleotide query sequence is at least 10 nucleic acid residues in length. In some embodiments, the random polynucleotide query sequence is at least 11 nucleic acid residues in length. In some embodiments, the random polynucleotide query sequence is at least 12 nucleic acid residues in length. In some embodiments, the random polynucleotide query sequence is at least 13 nucleic acid residues in length. In some embodiments, the random polynucleotide query sequence is at least 14 nucleic acid residues in length. In some embodiments, the random polynucleotide query sequence is at least 15 nucleic acid residues in length. In some embodiments, the random polynucleotide query sequence is at least 16 nucleic acid residues in length. In some embodiments, the random polynucleotide query sequence is at least 17 nucleic acid residues in length. In some embodiments, the random polynucleotide query sequence is at least 18 nucleic acid residues in length. In some embodiments, the random polynucleotide query sequence is at least 19 nucleic acid residues in length. In some embodiments, the random polynucleotide query sequence is at least 20 nucleic acid residues in length.

In some embodiments, the random polynucleotide query sequence is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length. In some embodiments, the random polynucleotide query sequence is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length. In some embodiments, the random polynucleotide query sequence is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length. In some embodiments, the random polynucleotide query sequence is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length. In some embodiments, the random polynucleotide query sequence is 5 nucleic acid residues in length. In some embodiments, the random polynucleotide query sequence is 6 nucleic acid residues in length. In some embodiments, the random polynucleotide query sequence is 7 nucleic acid residues in length. In some embodiments, the random polynucleotide query sequence is 8 nucleic acid residues in length. In some embodiments, the random polynucleotide query sequence is 9 nucleic acid residues in length. In some embodiments, the random polynucleotide query sequence is 10 nucleic acid residues in length. In some embodiments, the random polynucleotide query sequence is 11 nucleic acid residues in length. In some embodiments, the random polynucleotide query sequence is 12 nucleic acid residues in length. In some embodiments, the random polynucleotide query sequence is 13 nucleic acid residues in length. In some embodiments, the random polynucleotide query sequence is 14 nucleic acid residues in length. In some embodiments, the random polynucleotide query sequence is 15 nucleic acid residues in length. In some embodiments, the random polynucleotide query sequence is 16 nucleic acid residues in length. In some embodiments, the random polynucleotide query sequence is 17 nucleic acid residues in length. In some embodiments, the random polynucleotide query sequence is 18 nucleic acid residues in length. In some embodiments, the random polynucleotide query sequence is 19 nucleic acid residues in length. In some embodiments, the random polynucleotide query sequence is 20 nucleic acid residues in length.

In some embodiments, a random polynucleotide query sequence library is constructed using a primer with a random region. In some embodiments, the primer with a random region is a foldback primer. In some embodiments, a random polynucleotide query sequence library is constructed using the method described in Fan et. al. 2020 (doi: https://doi.org/10.1101/473207). In some embodiments, a random polynucleotide query sequence library is constructed with a foldback primer which comprises a random region. In some embodiments, the foldback primer is extended with a DNA polymerase or DNA polymerase fragment. In some embodiments, the DNA polymerase fragment is a Klenow fragment. In some embodiments, the DNA polymerase is a Taq polymerase.

In some embodiments, the random region of the primer or the foldback primer is 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, 40-mer, 41-mer, 42-mer, 43-mer, 44-mer, 45-mer, 46-mer, 47-mer, 48-mer, 49-mer, 50-mer, 51-mer, 52-mer, 54-mer, 55-mer, 56-mer, 57-mer, 58-mer, 59-mer, 60-mer, 61-mer, 62-mer, 63-mer, 64-mer, 65-mer, 66-mer, 67-mer, 68-mer, 69-mer, 70-mer, 81-mer, 82-mer, 83-mer, 84-mer, 85-mer, 86-mer, 87-mer, 88-mer, 89-mer, 90-mer, 91-mer, 92-mer, 93-mer, 94-mer, 95-mer, 96-mer, 97-mer, 98-mer, 99-mer, or 100-mer in length. In some embodiments, the random region of the foldback primer is a 10-mer in length. In some embodiments, the random region of the foldback primer is a 11-mer in length. In some embodiments, the random region of the foldback primer is a 12-mer in length. In some embodiments, the random region of the foldback primer is a 13-mer in length. In some embodiments, the random region of the foldback primer is a 14-mer length. In some embodiments, the random region of the foldback primer is 15-mer in length.

In some embodiments, the foldback primer comprises or consists of sequence of ATTCCGTCTCAAGTAANNNNNNNNNNATCATGGAGACGCACTGTTTTTTTCAGTGCGTCTCCATGA (SEQ ID NO: 15342), wherein “N” designates any nucleic acid residue.

In some embodiments, the resulting PCR product is ligated upstream of an expression cassette (e.g., encoding a reporter protein). In some embodiments, the resulting PCR product from a foldback primer is digested with restriction digestion enzyme(s) and ligated into an expression vector with an expression cassette (e.g., encoding a reporter protein).

Any suitable expression vector known in the art or described herein may be used. One of skill in the art would be well-equipped to construct an expression vector through standard recombinant techniques (see, for example, Sambrook et al., 2001(supra) and Ausubel et al., 1996 (supra), both incorporated herein by reference) for the expression of the random polynucleotide query sequences of the present disclosure. Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV, etc.), lentiviral vectors (e.g., derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, yeast-based vectors, bovine papilloma virus (BPV)-based vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

In some embodiments, the expression vector is pcircGFP-BsmBI vector (Fan et. al. 2020 (doi: https://doi.org/10.1101/473207). In some embodiments, the restriction digestion enzyme is BsmBI.

Any suitable reporter protein (e.g., a reporter protein encoded by the expression cassette) known in the art or described herein may be used. In some embodiments, cells containing a construct of the present disclosure (e.g., RNA polynucleotide having an IRES-like sequence and an expression cassette encoding a reporter protein) may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker (e.g., genes that confer resistance to neomycin, puromycin, hygromycin, DHFR, GPT, zeocin, and histidinol). Other types of markers including screenable markers such as GFP are also contemplated.

One of skill in the art would also know how to employ selection markers, possibly in conjunction with FACS analysis. The marker used is not believed to be important, so long as it is capable of being expressed simultaneously with the nucleic acid encoding a gene product. Further examples of selection and screenable markers are well known to one of skill in the art. In some embodiments, the reporter protein is a fluorescent protein. In some embodiments, the reporter protein is a green fluorescent protein (GFP).

### 8.2.2.1.b. Generating Overlapping Polynucleotide Subsequences within the Query Sequences

In some embodiments, a population of cells is contacted with the expression plasmid library. In some embodiments, the random polynucleotide query sequence expression plasmid library is transfected into a cell line to generate a population of cells. In some embodiments, the transfected cell line is a human cell line. In some embodiments, the transfected cell line is a human HEK293T cell line.

In some embodiments, the expression level of the reporter gene corresponding to each expression plasmid of the expression plasmid library is quantified. In some embodiments, the random polynucleotide query sequence expression plasmid library is transfected into a cell line to generate a population of cells and the levels of reporter protein expression from the plurality of cells within the population is determined.

In some embodiments, cells transfected with the plasmid library as described above are collected and sorted by reporter signal intensity (e.g., fluorescence intensity). In some embodiments, the reporter signal intensity is determined by fluorescence-activated cell sorting (FACS).

In some embodiments, the reporter signal intensity of the plurality of cells within the population is ranked. In some embodiments, the plurality of cells are separated in to two or more populations. In some embodiments, the plurality of cells are separated into at least a first population and a second population. In some embodiments, the plurality of cells are separated in to two populations (e.g., high and low reporter protein expression). In some embodiments, the plurality of cells are separated in to three populations (e.g., high, medium, and low reporter protein expression). In some embodiments, the plurality of cells are separated in to four populations (e.g., high, medium, low, and no reporter protein expression).

In some embodiments, a high expression population is determined by reporter protein signal intensity. In some embodiments, a high expression population consists of cells with reporter protein signal intensity in the top 0-0.1%, 0-0.2%, 0-0.3%, 0-0.4%, 0-0.5%, 0-0.6%, 0-0.7%, 0-0.8%, 0-0.9%, 0-1%, 0-1.1%, 0-1.2%, 0-1.3%, 0-1.4%, 0-1.5%, 0-1.6%, 0-1.7%, 0-1.8%, 0-1.9%, 0-2%, 0-2.1%, 0-2.2%, 0-2.3%, 0-2.4%, 0-2.5%, 0-2.6%, 0-2.7%, 0-2.8%, 0-2.9%, 0-3%, 0-3.1%, 0-3.2%, 0-3.3%, 0-3.4%, 0-3.5%, 0-3.6%, 0-3.7%, 0-3.8%, 0-3.9%, 0-4%, 0-4.1%, 0-4.2%, 0-4.3%, 0-4.4%, 0-4.5%, 0-4.6%, 0-4.7%, 0-4.8%, 0-4.9%, 0-5%, 0-5.1%, 0-5.2%, 0-5.3%, 0-5.4%, 0-5.5%, 0-5.6%, 0-5.7%, 0-5.8%, 0-5.9%, 0-6%, 0-6.1%, 0-6.2%, 0-6.3%, 0-6.4%, 0-6.5%, 0-6.6%, 0-6.7%, 0-6.8%, 0-6.9%, 0-7%, 0-7.1%, 0-7.2%, 0-7.3%, 0-7.4%, 0-7.5%, 0-7.6%, 0-7.7%, 0-7.8%, 0-7.9%, 0-8%, 0-8.1%, 0-8.2%, 0-8.3%, 0-8.4%, 0-8.5%, 0-8.6%, 0-8.7%, 0-8.8%, 0-8.9%, 0-9%, 0-9.1%, 0-9.2%, 0-9.3%, 0-9.4%, 0-9.5%, 0-9.6%, 0-9.7%, 0-9.8%, 0-9.9%, or 0-10% of the total population of cells rank ordered by reporter signal intensity. In some embodiments, the high expression population consists of cells with reporter protein signal intensity in the top about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% of the total population of cells rank ordered by reporter signal intensity. In some embodiments, the high expression population consists of cells with reporter protein signal intensity in the 0-10% of the total population of cells rank ordered by reporter signal intensity.

In some embodiments, a low expression population is determined by reporter protein signal intensity. In some embodiments, a low expression population consists of cells with reporter protein signal intensity in the bottom 0-0.1%, 0-0.2%, 0-0.3%, 0-0.4%, 0-0.5%, 0-0.6%, 0-0.7%, 0-0.8%, 0-0.9%, 0-1%, 0-1.1%, 0-1.2%, 0-1.3%, 0-1.4%, 0-1.5%, 0-1.6%, 0-1.7%, 0-1.8%, 0-1.9%, 0-2%, 0-2.1%, 0-2.2%, 0-2.3%, 0-2.4%, 0-2.5%, 0-2.6%, 0-2.7%, 0-2.8%, 0-2.9%, 0-3%, 0-3.1%, 0-3.2%, 0-3.3%, 0-3.4%, 0-3.5%, 0-3.6%, 0-3.7%, 0-3.8%, 0-3.9%, 0-4%, 0-4.1%, 0-4.2%, 0-4.3%, 0-4.4%, 0-4.5%, 0-4.6%, 0-4.7%, 0-4.8%, 0-4.9%, 0-5%, 0-5.1%, 0-5.2%, 0-5.3%, 0-5.4%, 0-5.5%, 0-5.6%, 0-5.7%, 0-5.8%, 0-5.9%, 0-6%, 0-6.1%, 0-6.2%, 0-6.3%, 0-6.4%, 0-6.5%, 0-6.6%, 0-6.7%, 0-6.8%, 0-6.9%, 0-7%, 0-7.1%, 0-7.2%, 0-7.3%, 0-7.4%, 0-7.5%, 0-7.6%, 0-7.7%, 0-7.8%, 0-7.9%, 0-8%, 0-8.1%, 0-8.2%, 0-8.3%, 0-8.4%, 0-8.5%, 0-8.6%, 0-8.7%, 0-8.8%, 0-8.9%, 0-9%, 0-9.1%, 0-9.2%, 0-9.3%, 0-9.4%, 0-9.5%, 0-9.6%, 0-9.7%, 0-9.8%, 0-9.9%, 0-10%, 0-11%, 0-12%, 0-13%, 0-14%, 0-15%, 0-16%, 0-17%, 0- 18%, 0-19%, 0-20%, 0-21%, 0-22%, 0-23%, 0-24%, 0-25%, 0-30%, 0-35%, 0-40%, 0-45%, 0-50%, 0-55%, 0-60%, 0-65%, 0-70%, 0-75%, 0-80%, 0-85%, 0-90%, 5-10%, 5-11%, 5-12%, 5-13%, 5-14%, 5-15%, 5-16%, 5-17%, 5-18%, 5-19%, 5-20%, 5-21%, 5-22%, 5-23%, 5-24%, 5-25%, 5-30%, 5-35%, 5-40%, 5-45%, 5-50%, 5-55%, 5-60%, 5-65%, 5-70%, 5-75%, 5-80%, 5-85%, 5-90%, 10-15%, 10-16%, 10-17%, 10-18%, 10-19%, 10-20%, 10-21%, 10-22%, 10-23%, 10-24%, 10-25%, 10-30%, 10-35%, 10-40%, 10-45%, 10-50%, 10-55%, 10-60%, 10-65%, 10-70%, 10-75%, 10-80%, 10-85%, or 10-90% of the total population of cells rank ordered by reporter signal intensity. In some embodiments, the low expression population consists of cells with reporter protein signal intensity in the bottom about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, or about 90% of the total population of cells ranked ordered by reporter signal intensity.

In some embodiments, a medium expression population is determined by reporter protein signal intensity. In some embodiments, the low expression population consists of cells with reporter protein signal intensity in between about 49%-51%, 48%-52%, 47%-53%, 46%-54%, 45%-55%, 44%-56%, 43%-57%, 42%-58%, 41%-59%, 40%-60%, 39%-61%, 38%-62%, 37%-63%, 36%-64%, 35%-65%, 34%-66%, 33%-67%, 32%-68%, 31%-69%, 30%-70%, 29%-71%, 28%-72%, 27%-73%, 26%-74%, or 25%-75% of the total population of cells ranked ordered by reporter signal intensity. In some embodiments, the selected expression population consists of cells with reporter protein signal intensity in between about 49%-51%, 48%-52%, 47%-53%, 46%-54%, 45%-55%, 44%-56%, 43%-57%, 42%-58%, 41%-59%, 40%-60%, 39%-61%, 38%-62%, 37%-63%, 36%-64%, 35%-65%, 34%-66%, 33%-67%, 32%-68%, 31%-69%, 30%-70%, 29%-71%, 28%-72%, 27%-73%, 26%-74%, or 25%-75% of the total population of cells ranked ordered by reporter signal intensity.

In some embodiments, a population with no expression is determined by reporter protein signal intensity. In some embodiments, a no expression population consists of cells with reporter protein signal intensity in the bottom 0-5%, 0-10%, 0-15%, 0-20%, 0-25%, 0-30%, 0-35%, 0-40%, 0-45%, or 0-50% of the total population of cells rank ordered by reporter signal intensity. In some embodiments, the low expression population consists of cells with reporter protein signal intensity in the bottom about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, or about 50% of the total population of cells ranked ordered by reporter signal intensity.

In some embodiments, a high expression population consists of cells with reporter protein signal intensity in the top 0-10% of the total population of cells ranked by reporter signal intensity and the low expression population consists of cells with reporter protein signal intensity in the bottom 0-90% of the total population of cells ranked by reporter signal intensity. In some embodiments, a high expression population consists of cells with reporter protein signal intensity in the top 0-5% of the total population of cells ranked by reporter signal intensity and the low expression population consists of cells with reporter protein signal intensity in the bottom 0-80% of the total population of cells ranked by reporter signal intensity. In some embodiments, a high expression population consists of cells with reporter protein signal intensity in the top 0-1% of the total population of cells ranked by reporter signal intensity and the low expression population consists of cells with reporter protein signal intensity in the bottom 0-75% of the total population of cells ranked by reporter signal intensity. In some embodiments, a high expression population consists of cells with reporter protein signal intensity in the top 0-0.5% of the total population of cells ranked by reporter signal intensity and the low expression population consists of cells with reporter protein signal intensity in the bottom 0-75% of the total population of cells ranked by reporter signal intensity. In some embodiments, a high expression population consists of cells with reporter protein signal intensity in the top about 0.5% of the total population of cells ranked by reporter signal intensity and the low expression population consists of cells with reporter protein signal intensity in the bottom about 75% of the total population of cells ranked by reporter signal intensity.

In some embodiments, a reference value for identifying a polynucleotide query sequence as an engineered IRES-like polynucleotide sequence is determined according to the reporter signal intensity, high expression population, and/or low expression population.

In some embodiments, a reference value for identifying a polynucleotide query sequence as an engineered IRES-like polynucleotide sequence is determined using the following formula: Reference Value=(23.75×length of the polynucleotide query sequence)−84.85−Z, wherein Z represents any number between 1 and 10000 and X represents the length of IRES-like polynucleotide sequence. In some embodiments, Z represents any number between 50 and 5000. In some embodiments, Z represents any number between 175 and 2500. In some embodiments, Z represents any number between 150 and 200.

In some embodiments, Z represents a number that is at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 1500, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, at least 9500 or at least 10000.

In some embodiments, Z represents a number that is about 1, about 2, about 3, about 4, about 5, about 6, about 7, about 8, about 9, about 10, about 20, about 30, about 40, about 50, about 60, about 70, about 80, about 90, about 100, about 200, about 300, about 400, about 500, about 600, about 700, about 800, about 900, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500 or about 10000.

In some embodiments, random polynucleotide query sequences are recovered from a sorted cell population and analyzed for enriched sequences. In some embodiments, the random sequences recovered from a sorted cell population is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length.

In some embodiments, X-mer random polynucleotide query sequences (i.e., random polynucleotide query sequences of X number of nucleic acid residues) are recovered from a high expression population and a low expression population. In some embodiments, X-mer random polynucleotide sequences are from a high expression population and a population with no expression.

In some embodiments, 4-mer to 100-mer, 4-mer to 90-mer, 4-mer to 80-mer, 4-mer to 70-mer, 4-mer to 60-mer, 4-mer to 50-mer, 4-mer to 40-mer, 4-mer to 30-mer, 4-mer to 20-mer, 4-mer to 19-mer, 4-mer to 18-mer, 4-mer to 17-mer, 4-mer to 16-mer, 4-mer to 15-mer, 4-mer to 14-mer, 4-mer to 13-mer, 4-mer to 12-mer, 4-mer to 11-mer, 4-mer to 10-mer, 4-mer to 9-mer, 4-mer to 8-mer, 4-mer to 7-mer, 5-mer to 100-mer, 5-mer to 90-mer, 5-mer to 80-mer, 5-mer to 70-mer, 5-mer to 60-mer, 5-mer to 50-mer, 5-mer to 40-mer, 5-mer to 30-mer, 5-mer to 20-mer, 5-mer to 19-mer, 5-mer to 18-mer, 5-mer to 17-mer, 5-mer to 16-mer, 5-mer to 15-mer, 5-mer to 14-mer, 5-mer to 13-mer, 5-mer to 12-mer, 5-mer to 11-mer, 5-mer to 10-mer, 5-mer to 9-mer, 5-mer to 8-mer, 5-mer to 7-mer, 6-mer to 100-mer, 6-mer to 90-mer, 6-mer to 80-mer, 6-mer to 70-mer, 6-mer to 60-mer, 6-mer to 50-mer, 6-mer to 40-mer, 6-mer to 30-mer, 6-mer to 20-mer, 6-mer to 19-mer, 6-mer to 18-mer, 6-mer to 17-mer, 6-mer to 16-mer, 6-mer to 15-mer, 6-mer to 14-mer, 6-mer to 13-mer, 6-mer to 12-mer, 6-mer to 11-mer, 6-mer to 10-mer, 6-mer to 9-mer, 6-mer to 8-mer, 6-mer to 7-mer, 7-mer to 100-mer, 7-mer to 90-mer, 7-mer to 80-mer, 7-mer to 70-mer, 7-mer to 60-mer, 7-mer to 50-mer, 7-mer to 40-mer, 7-mer to 30-mer, 7-mer to 20-mer, 7-mer to 19-mer, 7-mer to 18-mer, 7-mer to 17-mer, 7-mer to 16-mer, 7-mer to 15-mer, 7-mer to 14-mer, 7-mer to 13-mer, 7-mer to 12-mer, 7-mer to 11-mer, 7-mer to 10-mer, 7-mer to 9-mer, or 7-mer to 8-mer random polynucleotide query sequences (i.e., random polynucleotide query sequences of X number of nucleic acid residues) are recovered from a high expression population and a low expression population. In some embodiments, 4-mer to 100-mer, 4-mer to 90-mer, 4-mer to 80-mer, 4-mer to 70-mer, 4-mer to 60-mer, 4-mer to 50-mer, 4-mer to 40-mer, 4-mer to 30-mer, 4-mer to 20-mer, 4-mer to 19-mer, 4-mer to 18-mer, 4-mer to 17-mer, 4-mer to 16-mer, 4-mer to 15-mer, 4-mer to 14-mer, 4-mer to 13-mer, 4-mer to 12-mer, 4-mer to 11-mer, 4-mer to 10-mer, 4-mer to 9-mer, 4-mer to 8-mer, 4-mer to 7-mer, 5-mer to 100-mer, 5-mer to 90-mer, 5-mer to 80-mer, 5-mer to 70-mer, 5-mer to 60-mer, 5-mer to 50-mer, 5-mer to 40-mer, 5-mer to 30-mer, 5-mer to 20-mer, 5-mer to 19-mer, 5-mer to 18-mer, 5-mer to 17-mer, 5-mer to 16-mer, 5-mer to 15-mer, 5-mer to 14-mer, 5-mer to 13-mer, 5-mer to 12-mer, 5-mer to 11-mer, 5-mer to 10-mer, 5-mer to 9-mer, 5-mer to 8-mer, 5-mer to 7-mer, 6-mer to 100-mer, 6-mer to 90-mer, 6-mer to 80-mer, 6-mer to 70-mer, 6-mer to 60-mer, 6-mer to 50-mer, 6-mer to 40-mer, 6-mer to 30-mer, 6-mer to 20-mer, 6-mer to 19-mer, 6-mer to 18-mer, 6-mer to 17-mer, 6-mer to 16-mer, 6-mer to 15-mer, 6-mer to 14-mer, 6-mer to 13-mer, 6-mer to 12-mer, 6-mer to 11-mer, 6-mer to 10-mer, 6-mer to 9-mer, 6-mer to 8-mer, 6-mer to 7-mer, 7-mer to 100-mer, 7-mer to 90-mer, 7-mer to 80-mer, 7-mer to 70-mer, 7-mer to 60-mer, 7-mer to 50-mer, 7-mer to 40-mer, 7-mer to 30-mer, 7-mer to 20-mer, 7-mer to 19-mer, 7-mer to 18-mer, 7-mer to 17-mer, 7-mer to 16-mer, 7-mer to 15-mer, 7-mer to 14-mer, 7-mer to 13-mer, 7-mer to 12-mer, 7-mer to 11-mer, 7-mer to 10-mer, 7-mer to 9-mer, or 7-mer to 8-mer random polynucleotide sequences are from a high expression population and a population with no expression.

In some embodiments, X-mer random polynucleotide sequences (XN) are recovered from a population and are extended by appending random nucleotides (r) of the vector (e.g., DNA vector) sequence to the 5′ and/or 3′ ends to generate the polynucleotide query sequences (e.g., 5′-r-XN-r-3′). In some embodiments, the appended random nucleotides of the vector sequence is 1, 2, 3, 4, or 5 nucleic acid residues in length. In some embodiments, the appended random nucleotides of each end is 1 nucleic acid residues in length (e.g., 5′-1r-XN-1r-3′). In some embodiments, the appended random nucleotides is 2 nucleic acid residues in length (e.g., 5′-2r-XN-2r-3′).

As an illustration, in some embodiments, 10-mer random polynucleotide sequences are recovered from a high expression population and a low expression population. In some embodiments, 10-mer random polynucleotide sequences are from a high expression population and a population with no expression.

In some embodiments, 10-mer random polynucleotide sequences (1ON) are recovered from a population and are extended by appending random nucleotides (r) of the vector (e.g., DNA vector) sequence to the 5′ and/or 3′ ends to generate the polynucleotide query sequence (e.g., 5′-r-10N-r-3′). In some embodiments, the appended random nucleotides of the vector sequence is 1, 2, 3, 4, or 5 nucleic acid residues in length. In some embodiments, the appended random nucleotides of each end is 1 nucleic acid residues in length (e.g., 5′-1r-10N-1r-3′). In some embodiments, the appended random nucleotides is 2 nucleic acid residues in length (e.g., 5′-2r-10N-2r-3′).

In some embodiments, a series of overlapping polynucleotide fragment sequences (e.g., overlapping subsequences) are generated from the polynucleotide query sequence. In some embodiments, X-Y+1 number of overlapping polynucleotide fragment sequences within the polynucleotide query sequence, wherein X is the length of the polynucleotide query sequence, wherein each polynucleotide fragment sequence consists of Y nucleic acid residues in length, wherein the first position of each polynucleotide fragment sequence is n and the last position of the same polynucleotide fragment sequence is Y+n−1, and wherein n represents each positive integer between 1 and X−Y+1.

In some embodiments, the overlapping polynucleotide fragment sequence is greater than or equal to 2 nucleic acid residues in length. In some embodiments, the overlapping polynucleotide fragment sequence is 2-299 nucleic acid residues in length. In some embodiments, the overlapping polynucleotide fragment sequence is 2-200 nucleic acid residues in length. In some embodiments, the overlapping polynucleotide fragment sequence is 2-100 nucleic acid residues in length. In some embodiments, the overlapping polynucleotide fragment sequence is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length. In some embodiments, overlapping polynucleotide fragment sequence is 5 nucleic acid residues in length. In some embodiments, overlapping polynucleotide fragment sequence is 6 nucleic acid residues in length. In some embodiments, overlapping polynucleotide fragment sequence is 7 nucleic acid residues in length. In some embodiments, overlapping polynucleotide fragment sequence is 8 nucleic acid residues in length. In some embodiments, overlapping polynucleotide fragment sequence is 9 nucleic acid residues in length. In some embodiments, overlapping polynucleotide fragment sequence is 10 nucleic acid residues in length. In some embodiments, overlapping polynucleotide fragment sequence is 11 nucleic acid residues in length. In some embodiments, overlapping polynucleotide fragment sequence is 12 nucleic acid residues in length.

### 8.2.2.1.c. Determining an Enrichment Score

In some embodiments, the enrichment score for a polynucleotide fragment sequence is determined by: i) generating a expression plasmid library, wherein each expression plasmid of the library comprises a different polynucleotide fragment sequence and a reporter gene as described above, ii) contacting a population of cells with the expression plasmid library; iii) quantifying the expression level of the reporter gene corresponding to each expression plasmid of the expression plasmid library; iv) dividing the total population of cells into a first population and a second population based on the protein expression levels of iii); v) determining an enrichment score for the polynucleotide fragment sequence.

An enrichment score can be calculated using any suitable statistical analysis method known in the art or described herein. Exemplary methods for calculating an enrichment score include but are not limited to a Z-test, odd ratio, T-test, or Fisher's exact test. Exemplary methods for calculating an enrichment score are described in Wang, Z. et al. (Cell 119, 831 (2004)), each of which are incorporated herein by reference in their entirety.

In some embodiments, enrichment score of each overlapping polynucleotide fragment sequence within two populations are calculated using Z-test, thereby producing a Z-score. In some embodiments, the enrichment score (e.g., Z-score) of each overlapping polynucleotide fragment sequence is calculated using a Z-test. In some embodiments, the Z-test formula used to calculate a Z-score is shown in the following system of equations:

$$\begin{matrix}Z - Score = \frac{{f}_{1} - {f}_{2}}{\sqrt{( \frac{1}{{N}_{1}} + \frac{1}{{N}_{2}} ) P ( 1 - P )}} \\ P = \frac{{N}_{1} {f}_{1} - {N}_{2} {f}_{2}}{{N}_{1} + {N}_{2}}\end{matrix}$$

wherein f₁ is the frequency of a certain sequence in population 1, f₂ is the frequency of the same sequence in population 2, N₁ is the size of population 1, and N₂ is the size of population 2.

In some embodiments, the overlapping polynucleotide fragment sequence is a hexamer (6 nucleic acid residues in length or 6-mer). In some embodiments, f₁ is the frequency of each hexamer sequence in population 1, f₂ is the frequency of the same hexamer sequence in population 2, N₁ is the size of population 1 (i.e., the total number of hexamers in population 1), and N₂ is the size of population 2 (i.e., the total number of hexamers in population 2).

In some embodiments, the overlapping polynucleotide fragment sequence is a pentamer (5 nucleic acid residues in length or 5-mer). In some embodiments, f₁ is the frequency of each pentamer sequence in population 1, f₂ is the frequency of the same pentamer sequence in population 2, N₁ is the size of population 1 (i.e., the total number of pentamers in population 1), and N₂ is the size of population 2 (i.e., the total number of pentamers in population 2).

In some embodiments, the overlapping polynucleotide fragment sequence is a pentamer (5 nucleic acid residues in length or 5-mer). In some embodiments, f₁ is the frequency of each pentamer sequence in population 1, f₂ is the frequency of the same pentamer sequence in population 2, N₁ is the size of population 1, and N₂ is the size of population 2.

In some embodiments, population 1 is a high expression population described herein. In some embodiments, population 1 is a medium expression population described herein. In some embodiments, population 1 is a low expression population described herein. In some embodiments, population 1 is a no expression population described herein.

In some embodiments, population 2 is a high expression population described herein. In some embodiments, population 2 is a medium expression population described herein. In some embodiments, population 2 is a low expression population described herein. In some embodiments, population 2 is a no expression population described herein.

In some embodiments, population 1 is a high expression population described herein, and population 2 is a medium expression population described herein. In some embodiments, population 1 is a high expression population described herein, and population 2 is a low expression population described herein. In some embodiments, population 1 is a high expression population described herein, and population 2 is a no expression population described herein.

In some embodiments, population 1 is a medium expression population described herein, and population 2 is a low expression population described herein. In some embodiments, population 1 is a medium expression population described herein, and population 2 is a no expression population described herein. In some embodiments, population 1 is a medium expression population described herein, and population 2 is a high expression population described herein.

In some embodiments, population 1 is a low expression population described herein, and population 2 is a no expression population described herein. In some embodiments, population 1 is a low expression population described herein, and population 2 is a medium expression population described herein. In some embodiments, population 1 is a low expression population described herein, and population 2 is a high expression population described herein.

In some embodiments, population 1 is a no expression population described herein, and population 2 is a low expression population described herein. In some embodiments, population 1 is a no expression population described herein, and population 2 is a medium expression population described herein. In some embodiments, population 1 is a no expression population described herein, and population 2 is a high expression population described herein.

Exemplary overlapping polynucleotide fragment sequences (e.g., pentamers) and enrichment scores (e.g., Z-scores) are shown in Table 1.

As one of skill in the art will appreciate, rank ordering of overlapping polynucleotide fragments described herein provides a method whereby functional synthetic IRES-like sequences can be designed or generated. Without wishing to be bound by theory, it is believed that the inclusion of a high frequency of top ranked polynucleotide fragment motifs within a synthetic IRES-like sequence will result in a higher probability of IRES-like function. Top ranked polynucleotide fragment motifs within the IRES-like sequence include but are not limited to SEQ ID NOs: 1, 2, 3, 4, 5, 6, 8, 9, 10, 11, 12, 13, 14, 15, 17, 22, 23, 25, 58, 62.

Conversely, without wishing to be bound by theory, it is believed that the inclusion of a high frequency of the bottom ranked polynucleotide fragment motifs within a synthetic IRES-like sequence will result in a lower probability of IRES-like function.

### 8.2.2.1.d. Determining a Numerical Score of a Polynucleotide Sequence and Identifying an IRES-Like Sequence

In some embodiments, a numerical score for a polynucleotide query sequence is determined by summing the enrichment scores (e.g., Z-scores) for each overlapping polynucleotide fragment sequence. In some embodiments, a numerical score for a polynucleotide query sequence is determined by averaging the enrichment scores (e.g., Z-scores) for each overlapping polynucleotide fragment sequence.

In some embodiments, the numerical scores are normally distributed according to the following system of equations z=(X_{numerical score}−μ)/σ=2.06; and thus X=z*σ+μ. In some embodiments, a polynucleotide query sequence having a σ greater or equal to z are identified as an IRES-like sequence.

In some embodiments, the polynucleotide query sequence having a σ less than or equal to X are identified as a non-IRES sequence.

In some embodiments, a polynucleotide query sequence having a numerical score greater or equal to 0 are identified as IRES-like sequences. In some embodiments, a polynucleotide query sequence having a numerical score greater than or equal to 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300 or 2500 are identified as IRES-like sequences.

In some embodiments, a polynucleotide query sequence having a numerical score less than 0 is identified as a non-IRES sequence. In some embodiments, a polynucleotide query sequence having a numerical score smaller than 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300 or 2500 are identified as IRES-like sequences.

In some embodiments, the polynucleotide query sequences having rank ordered numerical scores in the top 5% of all equivalent length polynucleotide query sequences are considered as IRES-like sequences. In some embodiments, the polynucleotide query sequences having rank ordered numerical scores in the top 10% of all equivalent length polynucleotide query sequences are considered as IRES-like sequences. In some embodiments, the polynucleotide query sequences having rank ordered numerical scores in the top 15% of all equivalent length polynucleotide query sequences are considered as IRES-like sequences. In some embodiments, the polynucleotide query sequences having rank ordered numerical scores in the top 20% of all equivalent length polynucleotide query sequences are considered as IRES-like sequences. In some embodiments, the polynucleotide query sequences having rank ordered numerical scores in the top 25% of all equivalent length polynucleotide query sequences are considered as IRES-like sequences.

In some embodiments, the polynucleotide query sequences having rank ordered numerical scores in the bottom 5% of all equivalent length polynucleotide query sequences are considered as non-IRES sequences. In some embodiments, the polynucleotide query sequences having rank ordered numerical scores in the bottom 10% of all equivalent length polynucleotide query sequences are considered as non-IRES sequences. In some embodiments, the polynucleotide query sequences having rank ordered numerical scores in the bottom 15% of all equivalent length polynucleotide query sequences are considered as non-IRES sequences. In some embodiments, the polynucleotide query sequences having rank ordered numerical scores in the bottom 20% of all equivalent length polynucleotide query sequences are considered as non-IRES sequences. In some embodiments, the polynucleotide query sequences having rank ordered numerical scores in the bottom 25% of all equivalent length polynucleotide query sequences are considered as non-IRES sequences.

In some embodiments, the polynucleotide query sequences having rank ordered numerical scores in the top 10% of all equivalent length polynucleotide query sequences are considered as IRES-like sequences. In some embodiments, the polynucleotide query sequences having rank ordered numerical scores in the top 5% of all equivalent length polynucleotide query sequences are considered as IRES-like sequences. In some embodiments, the polynucleotide query sequences having rank ordered numerical scores in the top 2% of all equivalent length polynucleotide query sequences are considered as IRES-like sequences. Exemplary IRES-like Sequences and Numerical Scores are shown in Tables 2-96, 110 and 112. Accordingly, exemplary IRES-like sequences of the disclosure include but are not limited to those listed in Tables 2-96, 110 and 112. In some embodiments, the IRES-like sequence comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the IRES-like sequences of Tables 2-96, 110 and 112 or a functional portion thereof.

### 8.2.2.2. Methods of Optimizing IRES-Like Sequences

Genetic algorithms are search algorithms that use random perturbations of a list of random subsets to generate new subjects (see Schmitt, Lothar M (2001), Theory of Genetic Algorithms, Theoretical Computer Science (259), pp. 1-61). Genetic algorithms represent one branch of the field of study called evolutionary computation, in that they imitate the biological processes of reproduction and natural selection to solve for the ‘fittest’ solutions (Goldberg, D. E. (1989), Genetic Algorithms in Search, Optimization, and Machine Learning. Reading: Addison-Wesley). Genetic algorithms are an iterative optimization procedure that repeatedly apply operators (such as selection, hybrid, and mutation) to a group of solutions until some criterion of convergence has been satisfied. Each iterative step where a new population is obtained is called a generation.

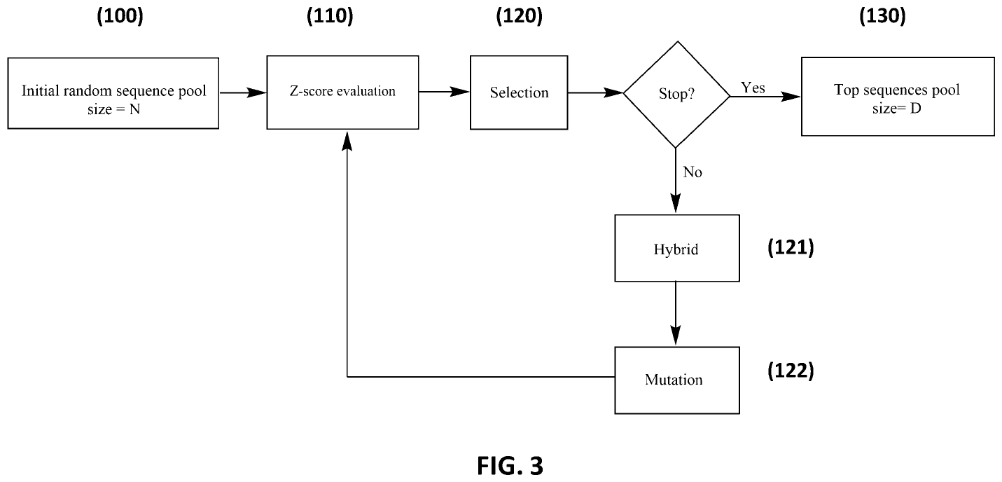

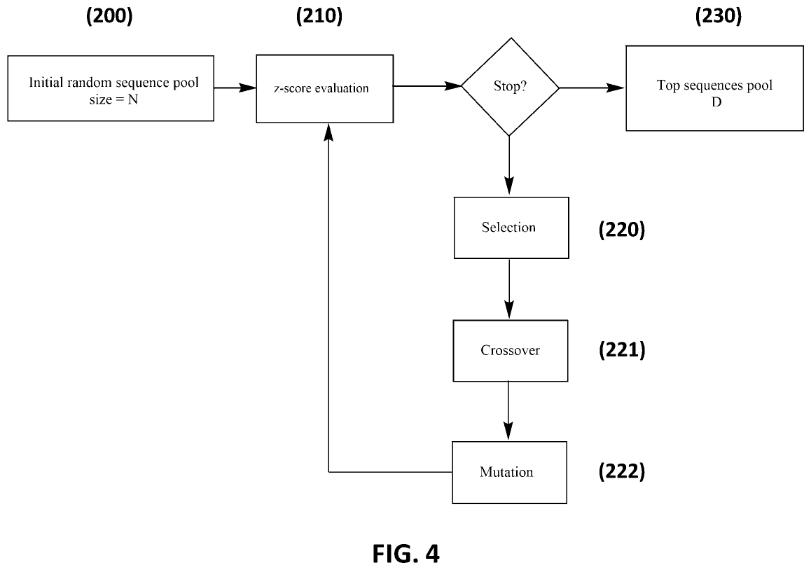

In some embodiments, a genetic algorithm is used to generate top sequences pool (FIG. 3, (130) and FIG. 4, (230)).

In some embodiments, a genetic algorithm procedure comprising the following steps as shown in FIG. 3: a) randomly generate an initial population with defined length as the current parent population, wherein the initial population has size=N (100); b) evaluate the objective function for each individual sequence in the initial population using an evaluation function (also known as a fitness function; a fitter sequence has higher score) (e.g., Z-score) (110); c) select top sequences to form a top sequence pool (size=D) (130); d) choose some of the sequences in the top sequence pool with a selection operator (the fitter a sequence is, the more likely is to be selected) (120); and e) generate an offspring population by a hybrid operator (121) and/or a mutation operator (122); and steps b)-e) cycle repeats.

In some embodiments, a genetic algorithm procedure comprising the following steps as shown in FIG. 4: a) randomly generate an initial population with defined length as the current parent population, wherein the initial population has size=N (200); b) evaluate the objective function for each individual sequence in the initial population using an evaluation function (also known as a fitness function; a fitter sequence has higher score) (e.g., Z-score) (210); c) select top sequences to form a top sequence pool (size=D) (230); d) choose some of the sequences in the top sequence pool with a selection operator (the fitter a sequence is, the more likely is to be selected) (220); and e) generate an offspring population by a crossover operator (221) and/or a mutation operator (222); and steps b)-e) cycle repeats.

In some embodiments, the initial population has a defined length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleic acid residues. In some embodiments, the initial population has a defined length in between 5-12 nucleic acid residues. In some embodiments, the initial population has a defined length in between 12-20 nucleic acid residues. In some embodiments, the initial population has a defined length in between 20-100 nucleic acid residues. In some embodiments, the initial population has a defined length in between 100-200 nucleic acid residues. In some embodiments, the initial population has a defined length in between 200-300 nucleic acid residues. In some embodiments, the initial population has a defined length in between 300-400 nucleic acid residues. In some embodiments, the initial population has a defined length in between 400-500 nucleic acid residues. In some embodiments, the initial population has a defined length in between 500-600 nucleic acid residues. In some embodiments, the initial population has a defined length in between 600-700 nucleic acid residues. In some embodiments, the initial population has a defined length in between 500-600 nucleic acid residues. In some embodiments, the initial population has a defined length in between 600-700 nucleic acid residues. In some embodiments, the initial population has a defined length in between 700-800 nucleic acid residues. In some embodiments, the initial population has a defined length in between 800-900 nucleic acid residues. In some embodiments, the initial population has a defined length in between 900-1000 nucleic acid residues.

In some embodiments, the genetic algorithms are iterated until the top sequence pool stabilizes and does not change for many generations. In some embodiments, the genetic algorithms are iterated until the top sequence pool stabilizes and does not change for 10, 15, 20, 25, 30, 35, 40, 45, 50, 55, 60, 65, 70, 75, 80, 85, 90, 95, or 100 generations.

In some embodiments, the genetic algorithms are iterated until the top sequence pool stabilizes and does not change for 20 generations.

In some embodiments, selection, hybrid, and mutation process continues until the number of offspring is the same as the initial population, so that the offspring generation is composed entirely of new offspring and the parent generation is completely replaced.

In some embodiments, selection, hybrid, and mutation process allow highly-scored sequences of the parent generation survive into the offspring generation.

In some embodiments, a genetic algorithm is used to select top-ranked sequences.

In some embodiments, a genetic algorithm is used to select bottom-ranked sequence.

In some embodiments, hybrids and mutations are applied to the sequence pool of the current generation to create the next generation. In some embodiments, random addition or deletion are applied to the sequence pool of the current generation to create the next generation. A hybrid step combines features from a pair of subsets to form a new subset. In one preferred embodiment, hybrid is an operator that is the same as crossover.

In one preferred embodiment, race search that uses a t-test to determine the probability of a subset being better than the current best subset by at least a small user-specified threshold is suitable.

In some embodiments, hybrids and mutations are applied to the sequence pool of the current generation to create the next generation. In some embodiments, random addition or deletion are applied to the sequence pool of the current generation to create the next generation. A hybrid step combines features from a pair of subsets to form a new subset. In one preferred embodiment, hybrid is an operator that is the same as crossover.

In one preferred embodiment, race search that uses a t-test to determine the probability of a subset being better than the current best subset by at least a small user-specified threshold is suitable.

### 8.3. Therapeutic Product

In some embodiments, the polynucleotide of the present disclosure encodes a therapeutic product.

In some embodiments, the therapeutic product is a polypeptide, a protein, an enzyme, an antibody, or a combination thereof. In some embodiments, the therapeutic product comprises one or more polypeptides, proteins, enzymes, antibodies, or a combination thereof.

In some embodiments, the therapeutic product is a protein or enzyme. In some embodiments, the protein or enzyme is associated with a genetic disease (e.g., a disease in which a genetic alteration (e.g., mutation) and/or protein dysregulation plays a role in the initiation, development, and/or manifestation of the disease).

In some embodiments, the therapeutic product is a polypeptide or protein. In some embodiments, the polypeptide or protein resembles a weakened or non-viable form of a disease-causing agent (e.g., an infectious agent such as pathogen), which can be selected from a microorganism, such as a bacterium, virus, fungus, parasite, or one or more components of such microorganism, such as toxins, proteins (e.g., surface proteins), and/or cell walls. In some embodiments, the therapeutic product is an antigen or agent which can stimulate the body's immune system to recognize the antigen or agent, generate antibodies against the antigen or agent, destroy the antigen or agent, and/or develop an immunological memory of the antigen or agent. In some embodiments, the therapeutic product is an antigen or agent which can induce and/or strengthen vaccine-induced memory and/or enable the immune system to respond rapidly and effectively to the antigen or agent in later encounters.

In some embodiments, the therapeutic product is derived from an infectious agent or a part, component, and/or product thereof (e.g., cell wall, genomic sequence, membrane, capsid, protein, lipid, glycan, toxin). In some embodiments, the infectious agent is selected from a virus, a bacterium, a fungus, a protozoan, and a helminth. In some embodiments, the infectious agent is selected a virus and a bacterium.

In some embodiments, the infectious agent is a virus selected from the group consisting of adenovirus; Herpes simplex, type 1; Herpes simplex, type 2; encephalitis virus, papillomavirus; Varicella-zoster virus; Epstein-Barr virus; human cytomegalovirus; human herpes virus, type 8; BK virus; JC virus; Smallpox; polio virus; human bocavirus; Parvovirus B19; human astrovirus; Norwalk virus; coxsackievirus; hepatitis A virus; hepatitis B virus; hepatitis C virus; hepatitis D virus; hepatitis E virus; rhinovirus; severe acute respiratory syndrome (SARS) virus; Yellow Fever virus; Dengue virus; West Nile virus; Rubella virus; Human Immunodeficiency virus (HIV); influenza virus; Guanarito virus; Junin virus; Lassa virus; Machupo virus; Sabia virus; Crimean-Congo hemorrhagic fever virus; Ebola virus; Marburg virus; measles virus; mumps virus; parainfluenza virus; respiratory syncytial virus (RSV); human metapneumovirus; Hendra virus; Nipah virus; rabies virus; Rotavirus; Orbivirus; Coltivirus; Banna virus; human Enterovirus; Hanta virus; West Nile virus; corona virus, SARS-associated coronavirus (SARS-CoV), SARS-CoV-2 virus (COVID-19 associated); Middle East respiratory syndrome corona virus; Japanese encephalitis virus; vesicular exanthernavirus; and eastern equine encephalitis.

In some embodiments, the infectious agent is a bacterium selected from tuberculosis (Mycobacterium tuberculosis), clindamycin-resistant Clostridium difficile, fluoroquinolon-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant Acinetobacter baumannii, and vancomycin-resistant Staphylococcus aureus (VRSA).

In some embodiments, the infectious agent is associated with humans, non-human primates, or other animals, such as birds, pigs, horses, dogs, cats, rabbits, mice, rats, cows, sheep, goats, and deer.

In some embodiments, the therapeutic product is an antibody. In some embodiments, the antibody includes, but is not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain, antibody heavy chain dimers, antibody light chain-heavy chain pairs, intrabodies, heteroconjugate antibodies, monovalent antibodies, antigen-binding fragments of full-length antibodies, and fusion proteins of the above. The antigen-binding fragments include, but are not limited to, single-domain antibodies (variable domain of heavy chain antibodies (VHHs) or nanobodies), Fabs, Fab's, F(ab′)₂s, Fds, Fvs, scFvs (single-chain variable fragments).

### 8.3.1. Methods of Therapeutic Product Production

The disclosure further provides a method of producing a protein in a cell, which comprises contacting a cell with the RNA polynucleotides described herein (e.g., circular RNA molecule), a DNA vector encoding or suitable for synthesizing the RNA polynucleotide, whereby the expression protein-coding nucleic acid sequence is translated and the protein is produced in the cell.

In some embodiments, a method of producing a protein in a cell comprises contacting a cell with an RNA polynucleotide (e.g., circular RNA) sequence described herein, or a vector comprising the RNA polynucleotide (e.g., circular RNA) sequence, under conditions whereby the protein-coding nucleic acid sequence is translated and the protein is produced in the cell. Also provided is a protein produced by the disclosed methods.

In some embodiments, production of the protein is tissue-specific. For example, the protein may be selectively produced in one or more of the following tissues: muscle, liver, kidney, brain, lung, skin, pancreas, blood, or heart.

In some embodiments, the protein is expressed recursively in the cell.

In some embodiments, the protein is produced in the cell for at least about 10%, at least about 20%, or at least about 30% longer than if the protein-coding nucleic acid sequence is provided to the cell using a viral vector encoding a linear RNA or as a linear RNA.

In some embodiments, the protein is produced in the cell at a level that is at least about 10%, at least about 20%, or at least about 30% higher than if the protein-coding nucleic acid sequence is provided to the cell using a viral vector or as a linear RNA.

In some embodiments, the RNA polynucleotides (e.g., circular RNA) described herein (including components thereof, such as the IRES-like sequences) facilitate cap-independent translation activity from the circRNA. Canonical translation via a cap-independent mechanism may be reduced in some human diseases. Accordingly, the use of circRNAs to express proteins may be particularly helpful for treating such diseases. In some embodiments, use of the circRNAs described herein facilitates cap-independent translation activity from the circRNA under conditions wherein cap-dependent translation is reduced or turned-off in a cell.

As discussed above, translation of the protein-coding nucleic acid sequence may occur in an infinite loop (i.e., recursively) when the IRES is in-frame with the protein-coding nucleic acid sequence and the protein-coding sequence lacks a stop codon. Thus, in some embodiments, the method of producing a protein in a cell produces a concatenated protein.

Any prokaryotic or eukaryotic host cell described herein may be contacted with the recombinant circRNA molecule or a vector comprising the circRNA molecule. The host cell may be a mammalian cell, such as a human cell. In some embodiments, the cell is in vivo. In some embodiments, the cell is in vitro. In some embodiments, the cell is ex vivo. In some embodiments, the cell is in a mammal, such as a human.

In some embodiments, regardless of cell type chosen, 5′ cap-dependent translation is impaired in the cell (e g., decreased, reduced, inhibited, or completely obliterated). In some embodiments, there is no substantial 5′ cap-dependent translation in the cell.

The recombinant circular RNA molecule, a DNA molecule encoding same, or vectors comprising same, may be introduced into a cell by any method, including, for example, by transfection, transformation, or transduction. The terms “transfection, “transformation,” and “transduction” are used interchangeably herein and refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); strontium phosphate DNA co-precipitation (Brash et al., Mol. Cell. Biol., 7: 2031-2034 (1987); and magnetic nanoparticle-based gene delivery (Dobson, J., Gene Ther, 13 (4): 283-7 (2006)).

### 8.3.2. Post-Translational Modification of Therapeutic Product

In some embodiments, the therapeutic product (for example, a protein) is post-translationally modified. In some embodiments, the post-translational modification is specific to the cell type, the tissue type, or the organ, where the therapeutic product is produced or delivered.

In some embodiments, the post-translational modification is acetylation, SUMOylation, glycosylation, tyrosine sulfation, phosphorylation, ADP-ribosylation, prenylation, myristoylation, palmitoylation, ubiquitination, sentrinization, and/or a ubiquitination-like protein modification.

In some embodiments, the therapeutic product is post-translationally modified in vitro, ex vivo, or in vivo.

In some embodiments, the therapeutic product is post-translationally modified upon expression in a cell (e.g., a primary cell, a cell line derived from a primary cell, an immortalized cell). In some embodiments, the cell is an immortalized cell (e.g., a human immortalized cell).

In some embodiments, the therapeutic product is post-translationally modified in a living organism (e.g., a bacterium, an animal).

In some embodiments, the therapeutic product is post-translationally modified in an animal (e.g., an animal described herein). In some embodiments, the therapeutic product is post-translationally modified in a human.

In some embodiments, the post-translation modification can be detected by techniques known in the art, including by gel electrophoresis, Western blotting, Eastern blotting, immunoprecipitation, mass spectrometry, chromatography, and flow cytometry. Analysis of modified proteins is typically performed by electrophoresis and autoradiography, with specificity enhanced by immunoprecipitation of proteins of interest prior to electrophoresis.

In some embodiments, detection of the post-translation modification may include in vivo labeling of cellular substrate pools with radioactive substrate or substrate precursor molecules, which result in incorporation of radiolabeled moieties, including, but not limited to, phosphate, fatty acyl (e.g., myristoyl or palmitoyl), sentrin, methyl, acetyl, hydroxyl, iodine, flavin, ubiquitin or ADP-ribosyls, to the therapeutic product. In some embodiments, detection of the post-translation modification may include enzymatic incorporation of a labeled moiety into the therapeutic product in vitro to estimate the state of modification in vivo. In some embodiments, the labeled moiety includes but is not limited to, a radioactive isotope, a luminescent enzyme (e.g., horseradish peroxidase, HRP), or a fluorescent label.

In some embodiments, the post-translation modification can be detected by analyzing the alteration in electrophoretic mobility of a modified therapeutic product compared with an unmodified therapeutic product.

In some embodiments, the post-translation modification can be detected by thin-layer chromatography of radiolabeled fatty acids extracted from a therapeutic product.

In some embodiments, the post-translation modification can be detected by partitioning of a therapeutic product into detergent-rich or detergent layer by phase separation, and the effects of enzyme treatment of the therapeutic product on the partitioning between aqueous and detergent-rich environments.

### 8.4. RNA Polynucleotides

In some embodiments, provided herein is an RNA polynucleotide comprising an engineered translation initiation element (TI) comprising an IRES-like polynucleotide sequence.

In some embodiments, provided herein is an RNA polynucleotide comprising a construct of Formula I: 5′-(A1)₀₋₁-(L)ₙ-TI-(L)ₙ-Z1-(L)ₙ-(B1)₀₋₁-3′ (I).

In some embodiments, in Formula I, TI is an engineered translation initiation element comprising an IRES-like polynucleotide sequence; Z1 is an expression sequence encoding a therapeutic product; each L is independently a linker sequence; and n is a positive integer (e.g., an integer selected from 0 to 2).

In some embodiments, in Formula I: A1 and B1 are each independently a sequences capable of circularizing the RNA polynucleotide. In some embodiments, in Formula I: A1 and B1 are a pair of homologous sequences capable of spontaneous cleavage and circularization of the RNA polynucleotide.

In some embodiments, in Formula I: A1 and B1 each independently comprise a nucleotide derivative capable of joining the 5′ end and the 3′ end via a 3′ to 5′ phosphodiester linkage for circularization of the RNA polynucleotide.

In some embodiments, in Formula I: A1 and B1 each independently comprise a nucleotide triphosphate derivative capable of joining the 5′ end and the 3′ end via a 3′ to 5′ phosphodiester linkage for circularization of the RNA polynucleotide.

In some embodiments, provided herein is an RNA polynucleotide comprising a construct of Formula II: 5′-(A1)₀₋₁-(L)ₙ-Z1_{B}-(L)ₙ-TI-(L)ₙ-Z1_{A}-(L)ₙ-(B1)₀₋₁-3′ (11).

In some embodiments, in Formula II, TI is an engineered translation initiation element comprising an IRES-like polynucleotide sequence; Z1_{A} is a first portion of an expression sequence encoding a therapeutic product; Z1_{B} is a second portion of the expression sequence encoding a therapeutic product; each L is independently a linker sequence; and n is a positive integer (e.g., an integer selected from 0 to 2).

In some embodiments, in Formula II: A1 and B1 are each independently a sequences capable of circularizing the RNA polynucleotide. In some embodiments, in Formula I: A1 and B1 are a pair of homologous sequences capable of spontaneous cleavage and circularization of the RNA polynucleotide.

In some embodiments, in Formula II: A1 and B1 each independently comprise a nucleotide derivative capable of joining the 5′ end and the 3′ end via a 3′ to 5′ phosphodiester linkage for circularization of the RNA polynucleotide.

In some embodiments, in Formula II: A1 and B1 each independently comprise a nucleotide triphosphate derivative capable of joining the 5′ end and the 3′ end via a 3′ to 5′ phosphodiester linkage for circularization of the RNA polynucleotide.

In some embodiments, provided herein is an RNA polynucleotide comprising a construct of Formula III: 5′-(3′ intron fragment)-(E2)-(L)ₙ-TI-(L)ₙ-Z1-(L)ₙ-(E1)-(5′ intron fragment)-3′ (III).

In some embodiments, in Formula III, TI is an engineered translation initiation element comprising an IRES-like polynucleotide sequence; Z1 is an expression sequence encoding a therapeutic product; each L is independently a linker sequence; and n is a positive integer (e.g., an integer selected from 0 to 2).

In some embodiments, in Formula III, the 5′ intron fragment and the 3′ intron fragment are each a fragment of a group II intron, wherein the 5′ intron fragment is located on the 5′ side of the 3′ intron fragment in the group II intron.

In some embodiments, in Formula III, the E1 is a 5′ adjacent exon fragment of the group II intron, which is ≥0 nucleotides in length.

In some embodiments, in Formula III, the E2 is a 3′ adjacent exon fragment of the group II intron, which is 3 0 nucleotides in length.

In some embodiments, in Formula III, the E1 is a 5′ adjacent exon fragment of the group II intron, which is 3 0 nucleotides in length; the E2 is a 3′ adjacent exon fragment of the group II intron, which is ≥0 nucleotides in length.

In some embodiments, provided herein is an RNA polynucleotide comprising a construct of Formula IV: 5′-(3′ intron fragment)-(E2)-(L)ₙ-Z1_{B}-(L)ₙ-TI-(L)ₙ-Z1_{A-}-(L)ₙ-(E1)-(5′ intron fragment)-3′ (IV).

In some embodiments, in Formula IV, TI is an engineered translation initiation element comprising an IRES-like polynucleotide sequence; Z1_{A} is a first portion of an expression sequence encoding a therapeutic product; Z1_{B} is a second portion of the expression sequence encoding a therapeutic product; each L is independently a linker sequence; and n is a positive integer (e.g., an integer selected from 0 to 2).

In some embodiments, in Formula IV, the 5′ intron fragment and the 3′ intron fragment are each a fragment of a group II intron, wherein the 5′ intron fragment is located on the 5′ side of the 3′ intron fragment in the group II intron.

In some embodiments, in Formula IV, the E1 is a 5′ adjacent exon fragment of the group II intron, which is ≥0 nucleotides in length.

In some embodiments, in Formula IV, the E2 is a 3′ adjacent exon fragment of the group II intron, which is ≥0 nucleotides in length.

In some embodiments, in Formula IV, the E1 is a 5′ adjacent exon fragment of the group II intron, which is ≥0 nucleotides in length; the E2 is a 3′ adjacent exon fragment of the group II intron, which is ≥0 nucleotides in length.

In some embodiments, provided herein is an RNA polynucleotide comprising a construct of Formula V: 5′-TI-(L)ₙ-Z1-3′ (V).

In some embodiments, in Formula V, TI is an engineered translation initiation element comprising an IRES-like polynucleotide sequence; Z1 is an expression sequence encoding a therapeutic product; each L is independently a linker sequence; and n is a positive integer (e.g., an integer selected from 0 to 2).

In some embodiments, in Formula III or IV, the RNA polynucleotide further comprises a 5′ homology arm at the 5′ end of the 3′ intron fragment.

In some embodiments, in Formula III or IV, the RNA polynucleotide further comprises a 3′ homology arm at the 3′ end of the 5′ intron fragment.

In some embodiments, in Formula III or IV, the RNA polynucleotide further comprises a 5′ homology arm at the 5′ end of the 3′ intron fragment, and a 3′ homology arm at the 3′ end of the 5′ intron fragment.

In some embodiments, in Formula III or IV, the E1 and the E2 are each independently 0 to 20 nucleotides in length.

In some embodiments, in Formula III or IV, the 5′ intron fragment and the 3′ intron fragment are obtained by segmenting a group II intron at an unpaired region into two fragments, wherein the unpaired region is preferably selected from a linear region between two adjacent domains of the group II intron and a loop region of a stem-loop structure of domain 4 of the group II intron.

In some embodiments, the 5′ homology arm of the present disclosure includes but is not limited to those listed in Table 109.

In some embodiments, the 5′ homology arm of the present disclosure comprises a nucleic acid sequence that is at least 90%, at least 91%, at least 92%, at least 93%, at least 94%, at least 95%, at least 96%, at least 97%, at least 98%, at least 99% or at least 100% identical to a 5′ homology arm sequence of Table 109 or a functional portion thereof. In some embodiments, the 5′ homology arm of the present disclosure comprises a combination of more than one (e.g., two, three, or four) nucleic acid sequence that is at least 90%, at least 91%, at least 92%, at least 93%, at least 94%, at least 95%, at least 96%, at least 97%, at least 98%, at least 99% or at least 100% identical to a 5′ homology arm sequence of Table 109 or a functional portion thereof. In some embodiments, the 5′ homology arm of the present disclosure comprises a nucleic acid sequence selected from Table 109 or a functional portion thereof. In some embodiments, the 5′ homology arm of the present disclosure comprises a combination of more than one (e.g., two, three, or four) nucleic acid sequence selected from Table 109 or a functional portion thereof.

In some embodiments, the 5′ homology arm of the present disclosure is a nucleic acid sequence that is at least 90%, at least 91%, at least 92%, at least 93%, at least 94%, at least 95%, at least 96%, at least 97%, at least 98%, at least 99% or at least 100% identical to a 5′ homology arm sequence of Table 109 or a functional portion thereof. In some embodiments, the 5′ homology arm of the present disclosure is a combination of more than one (e.g., two, three, or four) nucleic acid sequence that is at least 90%, at least 91%, at least 92%, at least 93%, at least 94%, at least 95%, at least 96%, at least 97%, at least 98%, at least 99% or at least 100% identical to a 5′ homology arm sequence of Table 109 or a functional portion thereof. In some embodiments, the 5′ homology arm of the present disclosure is a nucleic acid sequence selected from Table 109 or a functional portion thereof. In some embodiments, the 5′ homology arm of the present disclosure is a combination of more than one (e.g., two, three, or four) nucleic acid sequence selected from Table 109 or a functional portion thereof.

In some embodiments, the 3′ homology arm of the present disclosure includes but is not limited to those listed in Table 109.

In some embodiments, the 3′ homology arm of the present disclosure comprises a nucleic acid sequence that is at least 90%, at least 91%, at least 92%, at least 93%, at least 94%, at least 95%, at least 96%, at least 97%, at least 98%, at least 99% or at least 100% identical to a 3′ homology arm sequence of Table 109 or a functional portion thereof. In some embodiments, the 3′ homology arm of the present disclosure comprises a combination of more than one (e.g., two, three, or four) nucleic acid sequence that is at least 90%, at least 91%, at least 92%, at least 93%, at least 94%, at least 95%, at least 96%, at least 97%, at least 98%, at least 99% or at least 100% identical to a 3′ homology arm sequence of Table 109 or a functional portion thereof. In some embodiments, the 3′ homology arm of the present disclosure comprises a nucleic acid sequence selected from Table 109 or a functional portion thereof. In some embodiments, the 3′ homology arm of the present disclosure comprises a combination of more than one (e.g., two, three, or four) nucleic acid sequence selected from Table 109 or a functional portion thereof.

In some embodiments, the 3′ homology arm of the present disclosure is a nucleic acid sequence that is at least 90%, at least 91%, at least 92%, at least 93%, at least 94%, at least 95%, at least 96%, at least 97%, at least 98%, at least 99% or at least 100% identical to a 3′ homology arm sequence of Table 109 or a functional portion thereof. In some embodiments, the 3′ homology arm of the present disclosure is a combination of more than one (e.g., two, three, or four) nucleic acid sequence that is at least 90%, at least 91%, at least 92%, at least 93%, at least 94%, at least 95%, at least 96%, at least 97%, at least 98%, at least 99% or at least 100% identical to a 3′ homology arm sequence of Table 109 or a functional portion thereof. In some embodiments, the 3′ homology arm of the present disclosure is a nucleic acid sequence selected from Table 109 or a functional portion thereof. In some embodiments, the 3′ homology arm of the present disclosure is a combination of more than one (e.g., two, three, or four) nucleic acid sequence selected from Table 109 or a functional portion thereof.

In some embodiments, the E1 sequence of the present disclosure includes but is not limited to those listed in Table 103. In some embodiments, the E1 sequence comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the E1 sequences of Table 103 or a functional portion thereof. In some embodiments, the E1 sequence is a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the E1 sequences of Table 103 or a functional portion thereof.

In some embodiments, the E2 sequence of the present disclosure includes but is not limited to those listed in Table 104. In some embodiments, the E2 sequence comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the E2 sequences of Table 104 or a functional portion thereof. In some embodiments, the E2 sequence is a nucleic acid sequence that is at least 90%, at least 910%, at least 92%, at least 93%, at least 94%, at least 95%, at least 96%, at least 97%, at least 98%, at least 99% or at least 100% identical to the nucleic acid sequence of the E2 sequences of Table 104 or a functional portion thereof.

In some embodiments, the group II intron sequence of the present disclosure includes but is not limited to those listed in Table 105. In some embodiments, the group II intron sequence comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the group II intron sequences of Table 105 or a functional portion thereof. In some embodiments, the group II intron sequence is a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the group II intron sequences of Table 105 or a functional portion thereof.

In some embodiments, the 3′ intron fragment sequence of the present disclosure includes but is not limited to those listed in Table 106. In some embodiments, the 3′ intron fragment sequences comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the 3′ intron fragment sequences of Table 106 or a functional portion thereof. In some embodiments, the 3′ intron fragment sequences is a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the 3′ intron fragment sequences of Table 106 or a functional portion thereof.

In some embodiments, the 5′ intron fragment sequence of the present disclosure includes but is not limited to those listed in Table 107. In some embodiments, the 5′ intron fragment sequences comprises a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the 5′ intron fragment sequences of Table 107 or a functional portion thereof. In some embodiments, the 5′ intron fragment sequences is a nucleic acid sequence that is at least 90%, at least 91%, at least 92%, at least 93%, at least 94%, at least 95%, at least 96%, at least 97%, at least 98%, at least 99% or at least 100% identical to the nucleic acid sequence of the 5′ intron fragment sequences of Table 107 or a functional portion thereof.

In some embodiments, in any one of Formulae described herein, n is an integer selected from 0 to 5. In some embodiments, n is an integer selected from 0 to 2. In some embodiments, n is 2. In some embodiments, n is 1. In some embodiments, n is 0.

In some embodiments, in any one of Formulae described herein, the TI further comprises a second IRES-like polynucleotide sequence. In some embodiments, the first IRES-like polynucleotide sequence and the second IRES-like polynucleotide sequence are the same. In some embodiments, the first IRES-like polynucleotide sequence and the second IRES-like polynucleotide sequence are the different.

In some embodiments, in any one of Formulae described herein, the IRES-like polynucleotide sequence is determined by any one of the methods described herein. In some embodiments, the IRES-like polynucleotide sequence comprises a nucleic acid sequence of about or at least about 90%, 95%, 97%, 98%, 99% or 100% identical to the nucleic acid sequence of SEQ ID NO: 1025-14161 or 14412-15341. In embodiments, the IRES-like polynucleotide comprises a nucleic acid sequence of about or at least about 90%, 95%, 97%, 98%, 99% or 100% identical to the nucleic acid sequence of SEQ ID NO: 1, 2, 3, 4, 5, 6, 8, 9, 10, 11, 12, 13, 14, 15, 17, 22, 23, 25, 58, 62.

In some embodiments, in any one of Formulae described herein, the IRES-like sequence is greater than or equal to 3 residues in length. In some embodiments, the IRES-like sequence is 3-300 nucleic acid residues in length. In some embodiments, the IRES-like sequence is 3-200 nucleic acid residues in length. In some embodiments, IRES-like sequence is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 58, 59, 60, 61, 62, 63, 64, 65, 66, 67, 68, 69, 70, 81, 82, 83, 84, 85, 86, 87, 88, 89, 90, 91, 92, 93, 94, 95, 96, 97, 98, 99, or 100 nucleic acid residues in length. In some embodiments, the IRES-like sequence is 5 nucleic acid residues in length. In some embodiments, the IRES-like sequence is 6 nucleic acid residues in length. In some embodiments, the IRES-like sequence is 7 nucleic acid residues in length. In some embodiments, the IRES-like sequence is 8 nucleic acid residues in length. In some embodiments, the IRES-like sequence is 9 nucleic acid residues in length. In some embodiments, the IRES-like sequence is 10 nucleic acid residues in length. In some embodiments, the IRES-like sequence is 11 nucleic acid residues in length. In some embodiments, the IRES-like sequence is 12 nucleic acid residues in length.

In some embodiments, in any one of Formulae described herein, the TI (i.e., an engineered translation initiation element comprising an IRES-like polynucleotide sequence) further comprises an IRES sequence that is isolated or derived from a natural IRES sequence. In some embodiments, the natural IRES sequence comprises a nucleic acid sequence that is at least 90%, at least 91%, at least 92%, at least 93%, at least 94%, at least 95%, at least 96%, at least 97%, at least 98%, at least 99% or 100% identical to the nucleic acid sequence of any one of the natural IRES sequences of Tables 98-100 or a functional portion thereof.

### 8.4.1. Expression Sequence

In some embodiments, an RNA polynucleotide provided herein comprise a expression sequence encoding a therapeutic product (Z1). In some embodiments, the expression sequence encodes a reporter protein. In some embodiments, the expression sequence encodes a therapeutic protein. Exemplary expression sequences include but are not limited to those listed in Tables 101 and 102.

In some embodiments, an RNA polynucleotide comprises one expression sequence. In some embodiments, a polynucleotide comprises more than one expression sequence, e.g., 2, 3, 4, or 5 expression sequences.

In some embodiments, an RNA polynucleotide encodes a protein that is made up of subunits that are encoded by more than one gene. For example, the protein may be a heterodimer, wherein each chain or subunit of the protein is encoded by a separate gene. It is possible that more than one RNA polynucleotide is delivered in the transfer vehicle and each RNA polynucleotide encodes a separate subunit of the protein. Alternatively, a single RNA polynucleotide may be engineered to encode more than one subunit. In some embodiments, separate RNA polynucleotide molecules encoding the individual subunits may be administered in separate transfer vehicles.

### 8.4.2. Linkers

In some embodiments, an RNA polynucleotide provided herein comprises a linker sequence (L). In some embodiments, the linker sequence is 3-300 nucleic acid residues in length. In some embodiments the linker sequence is about 3-10, 10-20, 20-30, 30-40, 40-50, 50-60, 60-70, 70-80, 80-90, 90-100, 100-125, 125-150, 150-175, 175-200, 200-225, 225-250, 250-275 or 275-300 nucleic acid sequences in length. In some embodiments, the linker sequence is about 3N nucleic acid residues in length, wherein N is an integer selected from 1-100. In some embodiments, the linker sequence is about 3N nucleic acid residues in length, wherein N is an integer selected from 1-50. In some embodiments, the linker sequence is about 3N nucleic acid residues in length, wherein N is an integer selected from 1-20. In some embodiments, the linker sequence is about 3N nucleic acid residues in length, wherein N is an integer selected from 1-10. In some embodiments, the linker sequence is about 3N nucleic acid residues in length, wherein N is 1, 2, 3, 4 or 5. In some embodiments, the linker sequence is about 3N nucleic acid residues in length, wherein N is 1, 2 or 3. In some embodiments, the linker sequence is about 3 nucleic acid residues in length.

In some embodiments, the linker sequence comprises the nucleic acid sequence of RCC, wherein R is a guanine or an adenine. In some embodiments, the linker comprises the nucleic acid sequence of RCCRCC, wherein R is a guanine or an adenine. In some embodiments, the linker comprises the nucleic acid sequence of RCCRCCRCC, wherein R is a guanine or an adenine.

In some embodiments, the linker comprises a nucleic acid sequence that encodes a 5′UTR, 3′UTR, poly-A sequence, polyA-C sequence, poly-C sequence, poly-U sequence, poly-G sequence, ribosome binding site, aptamer, riboswitch, ribozyme, small RNA binding site, translation regulation elements (e.g., a Kozak sequence), a protein binding site (e.g., PTBP1 or HUR) a non-natural nucleotide, or a non-nucleotide chemical-linker sequence.

In some embodiments, the RNA polynucleotides provided herein comprise a 3′ UTR. In some embodiments, the 3′ UTR is from human beta globin, human alpha globin xenopus beta globin, xenopus alpha globin, human prolactin, human GAP-43, human eEF1a1, human Tau, human TNFa, dengue virus, hantavirus small mRNA, bunyavirus small mRNA, turnip yellow mosaic virus, hepatitis C virus, rubella virus, tobacco mosaic virus, human IL-8, human actin, human GAPDH, human tubulin, hibiscus chlorotic linsgspot virus, woodchuck hepatitis virus post translationally regulated element, sindbis virus, turnip crinkle virus, tobacco etch virus, or Venezuelan equine encephalitis virus.

In some embodiments, the RNA polynucleotides provided herein comprise a 5′ UTR. In some embodiments, the 5′ UTR is from human beta globin, Xenopus laevis beta globin, human alpha globin, Xenopus laevis alpha globin, rubella virus, tobacco mosaic virus, mouse Gtx, dengue virus, heat shock protein 70 kDa protein 1A, tobacco alcohol dehydrogenase, tobacco etch virus, turnip crinkle virus, or the adenovirus tripartite leader.

In some embodiments, the RNA polynucleotides provided herein comprises a polyA region. In some embodiments the polyA region is at least 30 nucleotides or at least 60 nucleotides in length.

In some embodiments, the RNA polynucleotide described herein is circularized via a ligation reaction. In some embodiments, the RNA polynucleotide described herein is circularized in the presence of a T4 ligase. In some embodiments, the RNA polynucleotide described herein the RNA polynucleotide is circularized in the absence of a T4 ligase.

In some embodiments, the RNA polynucleotide described herein is circularized via a splicing reaction. In some embodiments, the RNA polynucleotide described herein is circularized in the presence of a spliceosome. In some embodiments, the RNA polynucleotide described herein is circularized in the absence of a spliceosome.

In some embodiments, the RNA polynucleotide described herein is circularized via a self-splicing reaction.

### 8.4.3. Vectors, Linear RNA, Pre-Cursor RNA, and Circular RNA

In some embodiments, the RNA polynucleotide provided herein is a single stranded RNA. In some embodiments, the polynucleotide is a linear RNA. In some embodiments, provided herein is a precursor RNA. In some embodiments, provided the RNA polynucleotide is encoded by a vector. In some embodiments, the precursor RNA is a linear RNA produced by in vitro transcription of a vector provided herein.

In some embodiments, the RNA polynucleotide is circular RNA or is useful for making a circular RNA polynucleotide. In some embodiments, provided herein is a circular RNA. In some embodiments, the circular RNA is a circular RNA produced by a vector provided herein. In some embodiments, the circular RNA is circular RNA produced by circularization of a precursor RNA provided herein.

### Circular RNAs

Circular RNAs (also referred to as “circRNAs” or “cRNAs”) are single-stranded RNAs that are joined head to tail. circRNAs have been recognized as a pervasive class of noncoding RNAs in eukaryotic cells. Typically generated through back splicing, circRNAs are found to be very stable.

A circular RNA may be generated by any suitable method known in the art or disclosed herein. Exemplary methods of generating circular RNA are described in WO2021263124, Wesselhoeft, R. A. et al., Nat. Commun. 9, 2629 (2018), and Chinese Patent Application No. 202110594352.4, each of which are incorporated herein in its entirety.

Circular RNAs can be generated by any non-mammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

Alternatively, circular RNAs can be produced in vitro from a linear RNA by chemical or enzymatic ligation of the 5′ and 3′ ends of the RNA. Chemical ligation can be performed, for example, using cyanogen bromide (BrCN) or ethyl-3-(3-dimethylaminopropyl) carbodiimide (EDC) for activation of a nucleotide phosphomonoester group to allow phosphodiester bond formation (Sokolova, FEBS Lett, 232: 153-155 (1988); Dolinnaya et al., Nucleic Acids Res., 19: 3067-3072 (1991); Fedorova, Nucleosides Nucleotides Nucleic Acids, 15: 1137-1147 (1996)). Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl2).

In some embodiments, splint ligation may be used to generate circular RNAs. Splint ligation involves the use of an oligonucleotide splint that hybridizes with the two ends of a linear RNA to bring the ends of the linear RNA together for ligation. Hybridization of the splint, which can be either a deoxyribo-oligonucleotide or a ribooligonucleotide, orients the 5-phosphate and 3-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, is more efficient in some cases than enzymatic ligation if the structure of the hybridized splint-RNA complex interferes with enzymatic activity (see, e.g., Dolinnaya et al. Nucleic Acids Res, 2/(23): 5403-5407 (1993); Petkovic et al., Nucleic Acids Res, 43(4): 2454-2465 (2015)).

While circular RNAs generally are more stable than their linear counterparts, primarily due to the absence of free ends necessary for exonuclease-mediated degradation, additional modifications may be made to the recombinant circRNA described herein to further improve stability. Still other kinds of modifications may improve circularization efficiency, purification of circRNA, and/or protein expression from circRNA. For example, the recombinant circRNA may be engineered to include “homology arms” (i.e., 9-19 nucleotides in length placed at the 5′ and 3′ ends of a precursor RNA with the aim of bringing the 5′ and 3′ splice sites into proximity of one another), spacer sequences (i.e., linker sequences), and/or a phosphorothioate (PS) cap (Wesselhoeft et al., Nat. Commun., 9: 2629 (2018)). The recombinant circRNA also may be engineered to include 2′-O-methyl fluoro- or —O-methoxyethyl conjugates, phosphorothioate backbones, or 2′,4′-cyclic 2′-O-ethyl modifications to increase the stability (Holdt et al., Front Physiol., 9: 1262 (2018); Kratzfeldt et al., Nature, 438(7068): 685-9 (2005); and Crooke et al., CellMetab., 27(4): 714-739 (2018)). The recombinant circRNA molecule also may comprise one or more modifications that reduce the innate immunogenicity of the circRNA molecule in a host, such as at least one N6-methyladenosine (m⁶A).

In some embodiments, the recombinant circular RNA molecule is encoded by a nucleic acid that comprises at least two introns and at least one exon. In some embodiments, a DNA sequence encoding a circular RNA molecule comprises sequences that encode at least two introns and at least one exon. The term “exon,” as used herein, refers to a nucleic acid sequence present in a gene which is represented in the mature form of an RNA molecule after excision of introns during transcription. Exons may be translated into protein (e.g., in the case of messenger RNA (mRNA)). The term “intron,” as used herein, refers to a nucleic acid sequence present in a given gene which is removed by RNA splicing during maturation of the final RNA product. Introns are generally found between exons. During transcription, introns are removed from precursor messenger RNA (pre-mRNA), and exons are joined via RNA splicing. In some embodiments, the recombinant circular RNA molecule comprises a nucleic acid sequence which includes one or more exons and one or more introns.

Accordingly, circular RNAs can be generated by splicing of either an endogenous or exogenous intron, as described in WO 2017/222911, incorporated by reference herein in its entirety. As used herein, the term “endogenous intron” means an intron sequence that is native to the host cell in which the circRNA is produced. For example, a human intron is an endogenous intron when the circRNA is expressed in a human cell. An “exogenous intron” means an intron that is heterologous to the host cell in which the circRNA is generated. For example, a bacterial intron would be an exogenous intron when the circRNA is expressed in a human cell. Numerous intron sequences from a wide variety of organisms and viruses are known and include sequences derived from genes encoding proteins, ribosomal RNA (rRNA), or transfer RNA (tRNA). Representative intron sequences are available in various databases, including the Group I Intron Sequence and Structure Database (ma.whu.edu.cn/gissd/), the Database for Bacterial Group II Introns (webapps2.ucalgary.ca/-groupii/index.html), the Database for Mobile Group II Introns (fp.ucalgary.ca/group2introns), the Yeast Intron DataBase (emblS16 heidelberg.de/Extemallnfo/seraphin/yidb.html), the Ares Lab Yeast Intron Database (compbio. soe.ucsc. edu/yeast_introns.html), the U12 Intron Database (genome.crg.es/cgibin/ul2db/ul2db.cgi), and the Exon-Intron Database (bpg.utoledo.edu/-afedorov/lab/eid.html).

In some embodiments, the RNA polynucleotide (e.g., circular RNA) may be of any length or size. In some embodiments the RNA polynucleotide is between 300 and 10000, 400 and 9000, 500 and 8000, 600 and 7000, 700 and 6000, 800 and 5000, 900 and 5000, 1000 and 5000, 1100 and 5000, 1200 and 5000, 1300 and 5000, 1400 and 5000, and/or 1500 and 5000 nucleotides in length.

In some embodiments, the RNA polynucleotide (e.g., circular RNA) is at least 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, or 5000 nt in length. In some embodiments, the RNA polynucleotide is no more than 3000 nt, 3500 nt, 4000 nt, 4500 nt, 5000 nt, 6000 nt, 7000 nt, 8000 nt, 9000 nt, or 10000 nt in length.

In some embodiments, the RNA polynucleotide (e.g., circular RNA) is about 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, 5000 nt, 6000 nt, 7000 nt, 8000 nt, 9000 nt, or 10000 nt in length.

In some embodiments, the RNA polynucleotide (e.g., circular RNA) is at least 500, 600, 700, 800, 900, 1000, 1500, 2000, 2500, 3000, 3500, 4000, 4500, 5000, 5500, 6000, 6500, 7000, 7500, 8000, 8500, 9000, 9500 or 10000 nt in length. The RNA polynucleotide (e.g., circular RNA) can be unmodified, partially modified or completely modified.

In some embodiments, the circular RNA provided herein has higher functional stability than mRNA comprising the same expression sequence. In some embodiments, the circular RNA provided herein has higher functional stability than mRNA comprising the same expression sequence, 5moU modifications, an optimized UTR, a cap, and/or a polyA tail.

In some embodiments, the circular RNA polynucleotide provided herein has a functional half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a functional half-life of 5-80, 10-70, 15-60, and/or 20-50 hours. In some embodiments, the circular RNA polynucleotide provided herein has a functional half-life greater than (e.g., at least 1.5-fold greater than, at least 2-fold greater than) that of an equivalent linear RNA polynucleotide encoding the same protein. In some embodiments, functional half-life can be assessed through the detection of functional protein synthesis.

In some embodiments, the circular RNA polynucleotide provided herein has a half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a half-life of 5-80, 10-70, 15-60, and/or 20-50 hours. In some embodiments, the circular RNA polynucleotide provided herein has a half-life greater than (e.g., at least 1.5-fold greater than, at least 2-fold greater than) that of an equivalent linear RNA polynucleotide encoding the same protein.

In some embodiments, the circular RNA provided herein may have a higher magnitude of expression than equivalent linear mRNA, e.g., a higher magnitude of expression 24 hours after administration of RNA to cells. In some embodiments, the circular RNA provided herein has a higher magnitude of expression than mRNA comprising the same expression sequence, 5moU modifications, an optimized UTR, a cap, and/or a polyA tail. In some embodiments, the circular RNA provided herein may have higher stability than an equivalent linear mRNA. In some embodiments, this may be shown by measuring receptor presence and density in vitro or in vivo post electroporation, with time points measured over 1 week. In some embodiments, this may be shown by measuring RNA presence via qPCR or ISH.

In some embodiments, a circular RNA polynucleotide provided herein comprises modified RNA nucleotides and/or modified nucleosides. In some embodiments, the modified nucleoside is m⁵C (5-methylcytidine). In some embodiments, the modified nucleoside is m⁵U (5-methyluridine). In some embodiments, the modified nucleoside is m⁶A (N⁶-methyladenosine). In some embodiments, the modified nucleoside is s²U (2-thiouridine). In some embodiments, the modified nucleoside is Y (pseudouridine). In some embodiments, the modified nucleoside is Um (2′-O-methyluridine). In some embodiments, the modified nucleoside is m¹A (1-methyladenosine); m²A (2-methyladenosine); Am (2′-0-methyladenosine); ms² m⁶A (2-methylthio-N⁶-methyladenosine); i⁶A (N⁶-isopentenyladenosine); ms2i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladeno sine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-0-ribosyladenosine (phosphate)); I (inosine); m¹I (1-methylinosine); m¹hn (1,2′-O-dimethylinosine); m³C (3-methylcytidine); Cm (2′-0-methylcytidine); s²C (2-thiocytidine); ac⁴C (N⁴-acetylcytidine); (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-0-methylguanosine); m² 2G (N²,N²-dimethylguanosine); m²Gm (N²,2′-O-dimethylguanosine); m² aGm (N²,N²,2′-O-trimethylguanosine); Gr(p) (2′-0-ribosylguanosine(phosphate)); yW (wybutosine); oayW (peroxywybutosine); OHyW (hydroxy wybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQi (7-aminomethyl-7-deazaguanosine); G⁺ (archaeosine); D (dihydrouridine); m⁵Um (5,2′-0-dimethyluridine); s⁴U (4-thiouridine); m⁵s²U (5-methyl-2-thiouridine); s²Um (2-thio-2′-0-methyluridine); acp³U (3-(3-amino-3-carboxypropyl)uridine); ho⁵U (5-hydroxyuridine); mo⁵U (5-methoxyuridine); cmo⁵U (uridine 5-oxy acetic acid); mcmo⁵U (uridine 5-oxy acetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-0-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s2U (5-methylaminomethyl-2-thiouridine); mnm⁵se²U (5-methylaminomethyl-2-selenouridine); ncm⁵U (5-carbamoylmethyluridine); ncm^{S}Um (5-carbamoylmethyl-2′-O-methyluridine); cmnm⁵U (5-carboxymethylaminomethyluridine); cmnm⁵Um (5-carboxymethylaminomethyl-2′-0-methyluridine); cmnm⁵s²U (5-carboxymethylaminomethyl-2-thiouridine); m⁶₂A (N⁶,N⁶-dimethyladenosine); Im (2′-0-methylinosine); m⁴C (N⁴-methylcytidine); m⁴Cm (N⁴,2′-0-dimethylcytidine); hm⁵C (5-hydraxymethylcytidine); m³U (3-methyluridine); cm⁵U (5-carboxymethyluridine); m⁶Am (N⁶,2′-O-dimethyladenosine); m⁶₂Am (N⁶,N⁶,0-2′-trimethyladenosine); m²,7G (N²,7-dimethylguanosine); m^{2,2,7}G (N²,N²,7-trimethylguanosine); m³Um (3,2′-0-dimethyluridine); m⁵D (5-methyldihydrouridine); f⁵Cm (5-formyl-2′-O-methylcytidine); m′Gm (1,2′-0-dimethylguanosine); m′Am (1,2′-0-dimethyladenosine); rm⁵U (5-taurinomethyluridine); τm5s2U (5-taurinomethyl-2-thiouridine)); imG-14 (4-demethylwyosine); imG2 (isowyosine); or ac⁶A (N⁶-acetyladenosine).

In some embodiments, the modified nucleoside may include a compound selected from the group of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, I-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-i-methyl-pseudoisocytidine, 4-thio-i-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, I-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, I-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In some embodiments, the modifications are independently selected from the group consisting of 5-methylcytosine, pseudouridine and 1-methylpseudouridine.

In some embodiments, polynucleotides may be codon-optimized. A codon optimized sequence may be one in which codons in a polynucleotide encoding a therapeutic product have been substituted in order to increase the expression, stability and/or activity of the therapeutic product. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the IRES.

### 8.4.4. RNA Polynucleotide Production

The vectors provided herein can be made using standard techniques of molecular biology. For example, the various elements of the vectors provided herein can be obtained using recombinant methods, such as by screening cDNA and genomic libraries from cells, or by deriving the polynucleotides from a vector known to include the same.

The various elements of the vectors provided herein can also be produced synthetically, rather than cloned, based on the known sequences. The complete sequence can be assembled from overlapping oligonucleotides prepared by standard methods and assembled into the complete sequence. See, e.g., Edge, Nature (1981) 292:756; Nambair et al, Science (1984) 223 1299; and Jay et al, J. Biol. Chem. (1984) 259:631 1.

Thus, particular nucleotide sequences can be obtained from vectors harboring the desired sequences or synthesized completely, or in part, using various oligonucleotide synthesis techniques known in the art, such as site-directed mutagenesis and polymerase chain reaction (PCR) techniques where appropriate. One method of obtaining nucleotide sequences encoding the desired vector elements is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al, Proc. Natl. Acad. Sci. USA (1991) 88:4084-4088. Additionally, oligonucleotide-directed synthesis (Jones et al, Nature (1986) 54:75-82), oligonucleotide directed mutagenesis of preexisting nucleotide regions (Riechmann et al, Nature (1988) 332:323-327 and Verhoeyen et al., Science (1988) 239: 1534-1536), and enzymatic filling-in of gapped oligonucleotides using T4 DNA polymerase (Queen et al, Proc. Natl. Acad. Sci. USA (1989) 86: 10029-10033) can be used.

The precursor RNA provided herein can be generated by incubating a vector provided herein under conditions permissive of transcription of the precursor RNA encoded by the vector. For example, in some embodiments a precursor RNA is synthesized by incubating a vector provided herein that comprises an RNA polymerase promoter upstream of its 5′ duplex forming region and/or expression sequence with a compatible RNA polymerase enzyme under conditions permissive of in vitro transcription. In some embodiments, the vector is incubated inside of a cell by a bacteriophage RNA polymerase or in the nucleus of a cell by host RNA polymerase P.

In some embodiments, provided herein is a method of generating precursor RNA by performing in vitro transcription using a vector provided herein as a template (e.g., a vector provided herein with an RNA polymerase promoter positioned upstream of the 5′ homology region).

In some embodiments, the resulting precursor RNA can be used to generate circular RNA (e.g., a circular RNA polynucleotide provided herein).

Thus, in some embodiments provided herein is a method of making circular RNA. In some embodiments, the method comprises synthesizing precursor RNA by transcription (e.g., run-off transcription) using a vector provided herein as a template, and incubating the resulting precursor RNA in conditions suitable for circularization, to form circular RNA.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA.

### 8.5. Formulations and Delivery

In some embodiments, the polynucleotide or circular RNA disclosed herein can be formulated using liposomes, lipoplexes, lipid nanoparticles, polymer based delivery systems, and viral vectors. In some embodiments, the polynucleotide or circular RNA may be formulated in a lipid nanoparticle such as those described in WO2012170930, herein incorporated by reference in its entirety. In some embodiments, the lipid may be a cleavable lipid such as those described in WO2012170889, herein incorporated by reference in its entirety. In some embodiments, the pharmaceutical compositions of the polynucleotide or circular RNA may include at least one of the PEGylated lipids described in WO2012099755, herein incorporated by reference. In some embodiments, a lipid nanoparticle formulation may be formulated by the methods described in WO2011127255 or WO2008103276, each of which is herein incorporated by reference in its entirety. A lipid nanoparticle may be coated or associated with a co-polymer such as, but not limited to, a block co-polymer, such as a branched polyether-polyamide block copolymer described in WO2013012476, herein incorporated by reference in its entirety. Liposomes, lipoplexes, or lipid nanoparticles may be used to improve the efficacy of the polynucleotide or circular RNA directed protein production as these formulations may be able to increase cell transfection by the polynucleotide and circular RNA, increase the in vivo or in vitro half-life of the polynucleotide and circular RNA, and/or allow for controlled release.

In some embodiments, a polynucleotide or circular RNA disclosed herein encodes a protein that is made up of subunits that are encoded by more than one gene. For example, the protein may be a heterodimer, wherein each chain or subunit of the protein is encoded by a separate gene. It is possible that more than one polynucleotide or circular RNA molecule is delivered in the transfer vehicle and each polynucleotide or circular RNA encodes a separate subunit of the protein. Alternatively, a single polynucleotide or circular RNA molecule may be engineered to encode more than one subunit. In some embodiments, separate polynucleotide or circular RNA molecules encoding the individual subunits may be administered in separate transfer vehicles.

The present disclosure contemplates the discriminatory targeting of target cells and tissues by both passive and active targeting means. The phenomenon of passive targeting exploits the natural distribution patterns of a transfer vehicle in vivo without relying upon the use of additional excipients or means to enhance recognition of the transfer vehicle by target cells. For example, transfer vehicles which are subject to phagocytosis by the cells of the reticulo-endothelial system are likely to accumulate in the liver or spleen, and accordingly, may provide a means to passively direct the delivery of the compositions to such target cells.

Alternatively, the present disclosure contemplates active targeting, which involves the use of targeting moieties that may be bound (either covalently or non-covalently) to the transfer vehicle to encourage localization of such transfer vehicle at certain target cells or target tissues. For example, targeting may be mediated by the inclusion of one or more endogenous targeting moieties in or on the transfer vehicle to encourage distribution to the target cells or tissues. Recognition of the targeting moiety by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes). As provided herein, the composition can comprise a moiety capable of enhancing affinity of the composition to the target cell. Targeting moieties may be linked to the outer bilayer of the lipid particle during formulation or post-formulation. These methods are well known in the art. In addition, some lipid particle formulations may employ fusogenic polymers such as PEAA, hemagglutinin, other lipopeptides (see U.S. Pat. No. 6,417,326, which is incorporated herein by reference) and other features useful for in vivo and/or intracellular delivery. In other some embodiments, the compositions of the present disclosure demonstrate improved transfection efficacies, and/or demonstrate enhanced selectivity towards target cells or tissues of interest. Contemplated therefore are compositions which comprise one or more moieties (e.g., peptides, aptamers, oligonucleotides, a vitamin or other molecules) that are capable of enhancing the affinity of the compositions and their nucleic acid contents for the target cells or tissues. Suitable moieties may optionally be bound or linked to the surface of the transfer vehicle. In some embodiments, the targeting moiety may span the surface of a transfer vehicle or be encapsulated within the transfer vehicle. Suitable moieties and are selected based upon their physical, chemical or biological properties (e.g., selective affinity and/or recognition of target cell surface markers or features). Cell-specific target sites and their corresponding targeting ligand can vary widely. Suitable targeting moieties are selected such that the unique characteristics of a target cell are exploited, thus allowing the composition to discriminate between target and non-target cells. For example, compositions of the disclosure may include surface markers (e.g., apolipoprotein-B or apolipoprotein-E) that selectively enhance recognition of, or affinity to hepatocytes (e.g., by receptor-mediated recognition of and binding to such surface markers). As an example, the use of galactose as a targeting moiety would be expected to direct the compositions of the present disclosure to parenchymal hepatocytes, or alternatively the use of mannose containing sugar residues as a targeting ligand would be expected to direct the compositions of the present disclosure to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting moieties that have been conjugated to moieties present in the transfer vehicle (e.g., a lipid nanoparticle) therefore facilitate recognition and uptake of the compositions of the present disclosure in target cells and tissues. Examples of suitable targeting moieties include one or more peptides, proteins, aptamers, vitamins and oligonucleotides.

In some embodiments, the polynucleotide or circular RNA disclosed herein is formulated according to a process described in U.S. Pub. No. 20180153822. In some embodiments, the present disclosure provides a process of encapsulating the polynucleotide or circular RNA in lipid nanoparticles comprising the steps of forming lipids into pre-formed lipid nanoparticles (i.e., formed in the absence of RNA) and then combining the pre-formed lipid nanoparticles with RNA. In some embodiments, the formulation process results in an RNA formulation with higher potency (peptide or protein expression) and higher efficacy (improvement of a biologically relevant endpoint) both in vitro and in vivo with potentially better tolerability as compared to the same RNA formulation prepared without the step of preforming the lipid nanoparticles (e.g., combining the lipids directly with the RNA).

In some embodiments, transfer vehicles are formulated and/or targeted as described in Shobaki N et al., Int J Nanomedicine 2018; 13:8395-8410. In some embodiments, a transfer vehicle is made up of 3 lipid types. In some embodiments, a transfer vehicle is made up of 4 lipid types. In some embodiments, a transfer vehicle is made up of 5 lipid types. In some embodiments, a transfer vehicle is made up of 6 lipid types.

For certain cationic lipid nanoparticle formulations of RNA, in order to achieve high encapsulation of RNA, the RNA in buffer (e.g., citrate buffer) has to be heated. In those processes or methods, the heating is required to occur before the formulation process (i.e., heating the separate components) as heating post-formulation (post-formation of nanoparticles) does not increase the encapsulation efficiency of the RNA in the lipid nanoparticles. In contrast, in some embodiments, the order of heating of RNA does not appear to affect the RNA encapsulation percentage. In some embodiments, no heating (i.e. maintaining at ambient temperature) of one or more of the solution comprising the pre formed lipid nanoparticles, the solution comprising the RNA and the mixed solution comprising the lipid nanoparticle encapsulated RNA is required to occur before or after the formulation process.

RNA may be provided in a solution to be mixed with a lipid solution such that the RNA may be encapsulated in lipid nanoparticles. A suitable RNA solution may be any aqueous solution containing RNA to be encapsulated at various concentrations. For example, a suitable RNA solution may contain an RNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable RNA solution may contain an RNA at a concentration in a range from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml.

Typically, a suitable RNA solution may also contain a buffering agent and/or salt. Generally, buffering agents can include HEPES, ammonium sulfate, Tris, sodium bicarbonate, sodium citrate, sodium acetate, potassium phosphate or sodium phosphate In some embodiments, a suitable concentration of the buffering agent may be in a range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM.

Exemplary salts can include sodium chloride, magnesium chloride, and potassium chloride. In some embodiments, suitable concentration of salts in an RNA solution may be in a range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM.

In some embodiments, a suitable RNA solution may have a pH in a range from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5.

Various methods may be used to prepare an RNA solution suitable for the present disclosure. In some embodiments, RNA may be directly dissolved in a buffer solution described herein. In some embodiments, an RNA solution may be generated by mixing an RNA stock solution with a buffer solution prior to mixing with a lipid solution for encapsulation. In some embodiments, an RNA solution may be generated by mixing an RNA stock solution with a buffer solution immediately before mixing with a lipid solution for encapsulation.

According to the present disclosure, a lipid solution contains a mixture of lipids suitable to form transfer vehicles for encapsulation of RNA. In some embodiments, a suitable lipid solution is ethanol based. For example, a suitable lipid solution may contain a mixture of desired lipids dissolved in pure ethanol (i.e. 100% ethanol). In some embodiments, a suitable lipid solution is isopropyl alcohol based. In some embodiments, a suitable lipid solution is dimethylsulfoxide-based. In some embodiments, a suitable lipid solution is a mixture of suitable solvents including, but not limited to, ethanol, isopropyl alcohol and dimethylsulfoxide.

A suitable lipid solution may contain a mixture of desired lipids at various concentrations. In some embodiments, a suitable lipid solution may contain a mixture of desired lipids at a total concentration in a range from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml.

Any desired lipids may be mixed at any ratios suitable for encapsulating RNAs. In some embodiments, a suitable lipid solution contains a mixture of desired lipids including cationic lipids, helper lipids (e.g., non cationic lipids and/or cholesterol lipids) and/or PEGylated lipids. In some embodiments, a suitable lipid solution contains a mixture of desired lipids including one or more cationic lipids, one or more helper lipids (e.g., non cationic lipids and/or cholesterol lipids) and one or more PEGylated lipids.

In some embodiments, the polynucleotide or circular RNA disclosed herein is formulated using viral vectors. Viral vectors may be derived from a variety of viruses including adenovirus, adeno-associated virus, lentivirus (e.g., HIV, FIV, and EIAV), and herpes virus. Examples of commercially available viral vectors include pSilencer adeno (Ambion, Austin, Tex.) and pLenti6/BLOCK-iT™-DEST (Invitrogen, Carlsbad, Calif.). Selection of viral vectors, methods for expressing the polynucleotide or circular RNA from the vector and methods of delivering the viral vector are within the ordinary skill of one in the art. In some embodiments, the viral vector is a recombinant AAV (rAAV) vector, such as those known in the art (PMID: 30245471, PMID: 33614232).

The present disclosure also provides a delivery system comprising the polynucleotide or circular RNA disclosed herein. In some embodiments, the delivery system is any one of a liposome, a nanoparticle, a polymer based delivery system or a ligand-conjugate delivery system. In some embodiments, the ligand-conjugate delivery system comprises one or more of an antibody, a peptide, a sugar moiety, or a combination thereof.

In some embodiments, the delivery system of the present disclosure comprise nanoparticles comprising the polynucleotide or circular RNA disclosed herein.

In some embodiments, the nanoparticle comprises a polymer-based nanoparticle, a lipid-polymer based nanoparticle, a metal based nanoparticle, a carbon nanotube based nanoparticle, a nanocrystal or a polymeric micelle. In some embodiments, the polymer-based nanoparticle comprises a multiblock copolymer, a deblock copolymer, a polymeric micelle or a hyperbranched macromolecule. In some embodiments, the polymer-based nanoparticle comprises a multiblock copolymer a diblock copolymer. In some embodiments, the polymer-based nanoparticle is pH responsive. In some embodiments, the polymer-based nanoparticle further comprises a buffering component.

In some embodiments, the delivery system comprises a liposome. Liposomes are spherical vesicles having at least one lipid bilayer, and in some embodiments, an aqueous core. In some embodiments, the lipid bilayer of the liposome may comprise phospholipids. An exemplary but non-limiting example of a phospholipid is phosphatidylcholine, but the lipid bilayer may comprise additional lipids, such as phosphatidylethanolamine. Liposomes may be multilamellar, i.e. consisting of several lamellar phase lipid bilayers, or unilamellar liposomes with a single lipid bilayer. Liposomes can be made in a particular size range that makes them viable targets for phagocytosis. Liposomes can range in size from 20 nm to 100 nm, 100 nm to 400 nm, 1 μM and larger, or 200 nm to 3 μM. Examples of lipidoids and lipid-based formulations are provided in U.S. Pub. No. 20090023673. In some embodiments, the one or more lipids are one or more cationic lipids.

In some embodiments, the liposome or the nanoparticle of the present disclosure comprises a micelle. A micelle is an aggregate of surfactant molecules. An exemplary micelle comprises an aggregate of amphiphilic macromolecules, polymers or copolymers in aqueous solution, wherein the hydrophilic head portions contact the surrounding solvent, while the hydrophobic tail regions are sequestered in the center of the micelle.

In some embodiments, the nanoparticle comprises a nanocrystal. Exemplary nanocrystals are crystalline particles with at least one dimension of less than 1000 nanometers, preferably of less than 100 nanometers.

In some embodiments, the nanoparticle comprises a polymer based nanoparticle. In some embodiments, the polymer comprises a multiblock copolymer, a diblock copolymer, a polymeric micelle or a hyperbranched macromolecule. In some embodiments, the particle comprises one or more cationic polymers. In some embodiments, the cationic polymer is chitosan, protamine, polylysine, polyhistidine, polyarginine or poly(ethylene)imine. In some embodiments, the one or more polymers contain the buffering component, degradable component, hydrophilic component, cleavable bond component or some combination thereof.

In some embodiments, the nanoparticles or some portion thereof are degradable. In some embodiments, the lipids and/or polymers of the nanoparticles are degradable.

In some embodiments, any of these delivery systems of the present disclosure can comprise a buffering component. In some embodiments, any of the of the present disclosure can comprise a buffering component and a degradable component. In some embodiments, any of the of the present disclosure can comprise a buffering component and a hydrophilic component. In some embodiments, any of the of the present disclosure can comprise a buffering component and a cleavable bond component. In some embodiments, any of the of the present disclosure can comprise a buffering component, a degradable component and a hydrophilic component. In some embodiments, any of the of the present disclosure can comprise a buffering component, a degradable component and a cleavable bond component. In some embodiments, any of the of the present disclosure can comprise a buffering component, a hydrophilic component and a cleavable bond component. In some embodiments, any of the of the present disclosure can comprise a buffering component, a degradable component, a hydrophilic component and a cleavable bond component. In some embodiments, the particle is composed of one or more polymers that contain any of the aforementioned combinations of components.

In some embodiments, the delivery system comprises a ligand-conjugate delivery system. In some embodiments, the ligand-conjugate delivery system comprises one or more of an antibody, a peptide, a sugar moiety, lipid or a combination thereof

In some embodiments, the polynucleotide or circular RNA disclosed herein is conjugated to, complexed to, or encapsulated by the one or more lipids or polymers of the delivery system. In some embodiments, the polynucleotide or circular RNA can be encapsulated in the hollow core of a nanoparticle. Alternatively, or in addition, the polynucleotide or circular RNA can be incorporated into the lipid or polymer based shell of the delivery system, for example via intercalation. Alternatively, or in addition, the polynucleotide or circular RNA can be attached to the surface of the delivery system. In some embodiments, the polynucleotide or circular RNA is conjugated to one or more lipids or polymers of the delivery system, e.g., via covalent attachment.

In some embodiments, the ligand conjugate delivery system further comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment.

In some embodiments, the delivery system of the present disclosure comprises a polymer based delivery system. In some embodiments, polymer based delivery system comprises a blending polymer. In some embodiments, the blending polymer is a copolymer comprising a degradable component and hydrophilic component. In some embodiments, the degradable component of the blending polymer is a polyester, poly(ortho ester), poly(ethylene imine), poly(caprolactone), polyanhydride, poly(acrylic acid), polyglycolide or poly(urethane). In some embodiments, the degradable component of the blending polymer is poly(lactic acid) (PLA) or poly(lactic-co-glycolic acid) (PLGA). In some embodiments, the hydrophilic component of the blending polymer is a polyalkylene glycol or a polyalkylene oxide. In some embodiments, the polyalkylene glycol is polyethylene glycol (PEG). In some embodiments, the polyalkylene oxide is polyethylene oxide (PEO).

In some embodiments, the delivery system of the present disclosure is a polymer based nanoparticle. Polymer based nanoparticles comprise one or more polymers. In some embodiments, the one or more polymers comprise a polyester, poly(ortho ester), poly(ethylene imine), poly(caprolactone), polyanhydride, poly(acrylic acid), polyglycolide or poly(urethane). In some embodiments, the one or more polymers comprise poly(lactic acid) (PLA) or poly(lactic-co-glycolic acid) (PLGA). In some embodiments, the one or more polymers comprise poly(lactic-co-glycolic acid) (PLGA). In some embodiments, the one or more polymers comprise poly(lactic acid) (PLA). In some embodiments, the one or more polymers comprise polyalkylene glycol or a polyalkylene oxide. In some embodiments, the polyalkylene glycol is polyethylene glycol (PEG) or the polyalkylene oxide is polyethylene oxide (PEO).

In some embodiments, the polymer-based nanoparticle comprises poly(lactic-co-glycolic acid) PLGA polymers. In some embodiments, the PLGA nanoparticle further comprises a targeting agent, as described herein.

In some embodiments, the delivery system of the present disclosure is a nanoparticle of average characteristic dimension of less than about 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm or 20 nm. In some embodiments, the nanoparticle has an average characteristic dimension of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm. In some embodiments, the nanoparticle has an average characteristic dimension of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm.

In some embodiments, the target cells are deficient in a protein or enzyme of interest. For example, where it is desired to deliver a nucleic acid to a hepatocyte, the hepatocyte represents the target cell. In some embodiments, the compositions of the disclosure transfect the target cells on a discriminatory basis (i.e., do not transfect non-target cells). The compositions of the disclosure may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, NK cells, dendritic cells, macrophages, reticulocytes, leukocytes, granulocytes, tumor cells (including tumor cell lines, such as Hela, MCF7, PC3, A549, NCI-H727, and HCT-116), immortalized cells (such as MCF10A, HEK293, HEK293T) and primary cell lines (such as liver stellate cells, HPrEC, FHC).

The compositions of the disclosure may be prepared to preferentially distribute to target cells in a particular organ as described herein, such as in the heart, lung, kidney, liver, and spleen. In some embodiments, the compositions of the disclosure distribute into the cells of the liver to facilitate the delivery and the subsequent expression of the circular RNA comprised therein by the cells of the liver (e.g., hepatocytes). The targeted cells may function as a biological “reservoir” or “depot” capable of producing, and systemically excreting a functional protein or enzyme. Accordingly, in some embodiments, the transfer vehicle may target hepatocytes and/or preferentially distribute to the cells of the liver upon delivery. In some embodiments, following transfection of the target hepatocytes, the circular RNA loaded in the vehicle are translated and a functional protein product is produced, excreted and systemically distributed. In some embodiments, cells other than hepatocytes (e.g., lung, spleen, heart, ocular, or cells of the central nervous system) can serve as a depot location for protein production.

In some embodiments, the compositions of the disclosure facilitate a subject's endogenous production of one or more functional proteins and/or enzymes. In some embodiments, the transfer vehicles comprise circular RNA which encode a deficient protein or enzyme. Upon distribution of such compositions to the target tissues and the subsequent transfection of such target cells, the exogenous circular RNA loaded into the transfer vehicle (e.g., a lipid nanoparticle) may be translated in vivo to produce a functional protein or enzyme encoded by the exogenously administered circular RNA (e.g., a protein or enzyme in which the subject is deficient). Accordingly, the compositions of the present disclosure exploit a subject's ability to translate exogenously- or recombinantly-prepared circular RNA to produce an endogenously-translated protein or enzyme, and thereby produce (and where applicable excrete) a functional protein or enzyme. The expressed or translated proteins or enzymes may also be characterized by the in vivo inclusion of native post-translational modifications which may often be absent in recombinantly-prepared proteins or enzymes, thereby further reducing the immunogenicity of the translated protein or enzyme.

The administration of circular RNA encoding a deficient protein or enzyme avoids the need to deliver the nucleic acids to specific organelles within a target cell. Rather, upon transfection of a target cell and delivery of the nucleic acids to the cytoplasm of the target cell, the circular RNA contents of a transfer vehicle may be translated and a functional protein or enzyme expressed.

In some embodiments, a circular RNA comprises one or more miRNA binding sites. In some embodiments, a circular RNA comprises one or more miRNA binding sites recognized by miRNA present in one or more non-target cells or non-target cell types (e.g., Kupffer cells) and not present in one or more target cells or target cell types (e.g., hepatocytes). In some embodiments, a circular RNA comprises one or more miRNA binding sites recognized by miRNA present in an increased concentration in one or more non-target cells or non-target cell types (e.g., Kupffer cells) compared to one or more target cells or target cell types (e.g., hepatocytes). miRNAs are thought to function by pairing with complementary sequences within RNA molecules, resulting in gene silencing.

### 8.6. Pharmaceutical Compositions

In some embodiments, provided herein are compositions (e.g., pharmaceutical compositions) comprising a therapeutic agent provided herein. In some embodiments, the therapeutic agent is a circular RNA polynucleotide provided herein. In some embodiments the therapeutic agent is a vector provided herein. In some embodiments, the therapeutic agent is a cell comprising a circular RNA or vector provided herein. In some embodiments, the composition further comprises a pharmaceutically acceptable carrier. In some embodiments, the compositions provided herein comprise a therapeutic agent provided herein in combination with other pharmaceutically active agents or drugs. In a preferred embodiment, the pharmaceutical composition comprises a cell provided herein or populations thereof.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions provided herein.

In some embodiments, the pharmaceutical composition comprises a preservative. In some embodiments, suitable preservatives may include, for example, methylparaben, propylparaben, sodium benzoate, and benzalkonium chloride. Optionally, a mixture of two or more preservatives may be used. The preservative or mixtures thereof are typically present in an amount of about 0.0001% to about 2% by weight of the total composition.

In some embodiments, the pharmaceutical composition comprises a buffering agent. In some embodiments, suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

In some embodiments, the concentration of therapeutic agent in the pharmaceutical composition can vary, e.g., less than about 1%, or at least about 1%, 2%, 3%, 4%, 5%, 6%, 7%, 8%, 9% 10%, 15%, 20%, 25%, 30%, 35%, 40%, 45%, or about 50% or more by weight, and can be selected primarily by fluid volumes, and viscosities, in accordance with the particular mode of administration selected.

The following formulations for oral, aerosol, parenteral (e.g., subcutaneous, intravenous, intraarterial, intramuscular, intradermal, intraperitoneal, and intrathecal), and topical administration are merely exemplary and are in no way limiting. More than one route can be used to administer the therapeutic agents provided herein, and in some instances, a particular route can provide a more immediate and more effective response than another route.

Formulations suitable for oral administration can comprise or consist of (a) liquid solutions, such as an effective amount of the therapeutic agent dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the therapeutic agent with a flavorant, usually sucrose, acacia or tragacanth. Pastilles can comprise the therapeutic agent with an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In some embodiments, the therapeutic agents provided herein can be administered in a physiologically acceptable diluent in a pharmaceutical carrier, such as a sterile liquid or mixture of liquids including water, saline, aqueous dextrose and related sugar solutions, an alcohol such as ethanol or hexadecyl alcohol, a glycol such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agents and other pharmaceutical adjuvants.

Oils, which can be used in parenteral formulations in some embodiments, include petroleum, animal oils, vegetable oils, or synthetic oils. Specific examples of oils include peanut, soybean, sesame, cottonseed, corn, olive, petrolatum, and mineral oil. Suitable fatty acids for use in parenteral formulations include oleic acid, stearic acid, and isostearic acid. Ethyl oleate and isopropyl myristate are examples of suitable fatty acid esters.

Suitable soaps for use in some embodiments of parenteral formulations include fatty alkali metal, ammonium, and triethanolamine salts, and suitable detergents include (a) cationic detergents such as, for example, dimethyl dialkyl ammonium halides and alkyl pyridinium halides, (b) anionic detergents such as, for example, alkyl, aryl, and olefin sulfonates, alky, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

In some embodiments, the parenteral formulations will contain, for example, from about 0.5% to about 25% by weight of the therapeutic agent in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol, sorbitan fatty acid esters such as sorbitan monooleate, and high molecular weight adducts of ethylene oxide with a hydrophobic base formed by the condensation of propylene oxide with propylene glycol. The parenteral formulations can be presented in unit-dose or multi-dose sealed containers, such as ampoules or vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

In some embodiments, injectable formulations are provided herein. The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Toissel, 4th ed, pages 622-630 (1986)).

In some embodiments, topical formulations are provided herein. Topical formulations, including those that are useful for transdermal drug release, are suitable in the context of certain embodiments provided herein for application to skin. In some embodiments, the therapeutic agent alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

In some embodiments, the therapeutic agents provided herein can be formulated as inclusion complexes, such as cyclodextrin inclusion complexes, or liposomes. Liposomes can serve to target the therapeutic agents to a particular tissue. Liposomes also can be used to increase the half-life of the therapeutic agents. Many methods are available for preparing liposomes, as described in, for example, Szoka et al, Ann. Rev. Biophys. Bioeng., 9, 467 (1980) and U.S. Pat. Nos. 4,235,871, 4,501,728, 4,837,028, and 5,019,369.

In some embodiments, the therapeutic agents provided herein are formulated in time-released, delayed release, or sustained release delivery systems such that the delivery of the composition occurs prior to, and with sufficient time to cause, sensitization of the site to be treated. Such systems can avoid repeated administrations of the therapeutic agent, thereby increasing convenience to the subject and the physician, and may be particularly suitable for certain composition embodiments provided herein. In some embodiments, the compositions of the disclosure are formulated such that they are suitable for extended-release of the circRNA contained therein. Such extended-release compositions may be conveniently administered to a subject at extended dosing intervals. For example, in some embodiments, the compositions of the present disclosure are administered to a subject twice a day, daily or every other day. In some embodiments, the compositions of the present disclosure are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by a polynucleotide described herein is produced by a target cell for sustained amounts of time. For example, the protein may be produced for more than one hour, more than four, more than six, more than 12, more than 24, more than 48 hours, or more than 72 hours after administration. In some embodiments the therapeutic product is expressed at a peak level about six hours after administration. In some embodiments the expression of the therapeutic product is sustained at least at a therapeutic level. In some embodiments the therapeutic product is expressed at least at a therapeutic level for more than one, more than four, more than six, more than 12, more than 24, more than 48, or more than 72 hours after administration. In some embodiments, the therapeutic product is detectable at a therapeutic level in patient serum or tissue (e.g., liver or lung). In some embodiments, the level of detectable therapeutic product is from continuous expression from the circRNA composition over periods of time of more than one, more than four, more than six, more than 12, more than 24, more than 48, or more than 72 hours after administration.

In some embodiments, a protein encoded by a polynucleotide described herein is produced at levels above normal physiological levels. The level of protein may be increased as compared to a control. In some embodiments, the control is the baseline physiological level of the therapeutic product in a normal individual or in a population of normal individuals. In some embodiments, the control is the baseline physiological level of the therapeutic product in an individual having a deficiency in the relevant protein or polypeptide or in a population of individuals having a deficiency in the relevant protein or polypeptide. In some embodiments, the control can be the normal level of the relevant protein or polypeptide in the individual to whom the composition is administered. In some embodiments, the control is the expression level of the therapeutic product upon other therapeutic intervention, e.g., upon direct injection of the corresponding therapeutic product, at one or more comparable time points.

In some embodiments, the levels of a protein encoded by a polynucleotide described herein are detectable at 3 days, 4 days, 5 days, or 1 week or more after administration. Increased levels of secreted protein may be observed in the serum and/or in a tissue (e.g., liver or lung).

In some embodiments, the method yields a sustained circulation half-life of a protein encoded by a polynucleotide described herein. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more.

Many types of release delivery systems are available and known to those of ordinary skill in the art. They include polymer based systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono- di- and tri-glycerides; hydrogel release systems; sylastic systems; peptide based systems: wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

In some embodiments, the therapeutic agent can be conjugated either directly or indirectly through a linking moiety to a targeting moiety. Methods for conjugating therapeutic agents to targeting moieties is known in the art. See, for instance, Wadwa et al, J, Drug Targeting 3:111 (1995) and U.S. Pat. No. 5,087,616.

In some embodiments, the therapeutic agents provided herein are formulated into a depot form, such that the manner in which the therapeutic agent is released into the body to which it is administered is controlled with respect to time and location within the body (see, for example, U.S. Pat. No. 4,450,150). Depot forms of therapeutic agents can be, for example, an implantable composition comprising the therapeutic agents and a porous or non-porous material, such as a polymer, wherein the therapeutic agents are encapsulated by or diffused throughout the material and/or degradation of the non-porous material. The depot is then implanted into the desired location within the body and the therapeutic agents are released from the implant at a predetermined rate.

### 8.7. Methods of Use

In some aspects, provided herein is a method of treating and/or preventing a condition, e.g., cancer, comprising introducing pharmaceutical composition provided herein into a subject in need thereof (e.g., a subject with cancer). In some embodiments, the pharmaceutical composition comprises a circular RNA polynucleotide provided herein. In some embodiments, the pharmaceutical composition comprises a vector provided herein. In some embodiments, the pharmaceutical composition comprises a cell (e.g., human cell) comprising a polynucleotide provided herein (e.g., a circular RNA or a vector provided herein).

Thus, in some embodiments, provided herein are methods of treating and/or preventing a disease in a subject (e.g., mammalian subject, such as a human subject). Without being bound to a particular theory or mechanism, the circular RNA provided herein can be used to express therapeutic proteins for protein replacement therapy.

In some embodiments, the therapeutic agents provided herein are co-administered with one or more additional therapeutic agents (e.g., in the same pharmaceutical composition or in separate pharmaceutical compositions). In some embodiments, the therapeutic agent provided herein can be administered first and the one or more additional therapeutic agents can be administered second, or vice versa. Alternatively, the therapeutic agent provided herein and the one or more additional therapeutic agents can be administered simultaneously.

In some embodiments, the therapeutic agent is a cell or population of cells comprising a circular RNA or a vector provided herein that expresses a protein encoded by the circular RNA or vector. In some embodiments, the administered cells are allogeneic to the subject being treated. In some embodiments, the administered cells are autologous to the subject being treated.

In some embodiments, the subject is a mammal. In some embodiments, the mammal referred to herein can be any mammal, including, but not limited to, mammals of the order Rodentia, such as mice and hamsters, or mammals of the order Logomorpha, such as rabbits. The mammals may be from the order Carnivora, including Felines (cats) and Canines (dogs). The mammals may be from the order Artiodactyla, including Bovines (cows) and Swines (pigs), or of the order Perssodactyla, including Equines (horses). The mammals may be of the order Primates, Ceboids, or Simoids (monkeys) or of the order Anthropoids (humans and apes). Preferably, the mammal is a human.

Therapeutically effective amounts of RNA polynucleotides (e.g., circular RNAs) can be administered by a number of routes, including parenteral administration, for example, intravenous, intraperitoneal, intramuscular, intrasternal, or intraarticular injection, or infusion.

### 8.7.1. Gene Therapy

Compositions and methods are described for the administration of a therapeutically effective amount of an RNA polynucleotide or circular RNA disclosed herein to a subject (e.g., a human) to treat a disease or disorder, for example, a disease or disorder in which the therapeutic product is involved in initiation, development, and/or manifestation of the disease or disorder, including a disease or disorder associated with protein misfolding and/or protein degenerative disease and a disease or disorder associated with a genetic mutation. In some embodiments, the RNA polynucleotide or circular RNA described herein comprises an IRES-like sequence, an endogenous IRES sequence or a variant thereof, or a combination thereof. In some embodiments, the IRES-like sequence, the endogenous IRES sequence or a variant thereof, or the combination thereof is optimized, as disclosed herein, for the expression of a therapeutic product. In some embodiments, the RNA polynucleotide or circular RNA provided herein may be used, for example, in a gene therapy. In some embodiments, the RNA polynucleotide or circular RNA described herein may be used in combination with a CRISPR-Cas system, Zinc finger nuclease (ZFN), Transcription Activator-Like Effector Nuclease (TALEN), meganuclease, and Puf for RNA editing.

### 8.7.2. Vaccine

Compositions and methods are described herein for the administration of a therapeutically effective amount of an RNA polynucleotide or circular RNA disclosed herein to a subject (e.g., a human) to stimulate an immune reaction to an antigen or an agent (e.g., an infectious agent such as pathogen), generate antibodies against the antigen or agent, destroy the antigen or agent, and/or develop an immunological memory of the antigen or agent. In some embodiments, the RNA polynucleotide described herein comprises an IRES-like sequence, an endogenous IRES sequence or a variant thereof, or a combination thereof. In some embodiments, the RNA polynucleotide described herein comprises an expression sequence encoding a therapeutic product. In some embodiments, the IRES-like sequence, the endogenous IRES sequence or a variant thereof, or the combination thereof is optimized, as disclosed herein, for the expression of a therapeutic product in the subject. In some embodiments, the RNA polynucleotide described herein may be used directly for making a vaccine (e.g., an RNA vaccine).

### 8.7.3. Antibody-Based Therapy

In some embodiments, the therapeutic product is a polypeptide or protein. In some embodiments, the polypeptide or protein resembles a weakened or non-viable form of a disease-causing agent (e.g., pathogen), which can be selected from a microorganism, such as a bacterium, virus, fungus, parasite, or one or more components of such microorganism, such as toxins, proteins (e.g., surface proteins), and/or cell walls. In some embodiments, the therapeutic product is an antigen or agent which can stimulate the body's immune system to recognize the antigen or agent, generate antibodies against the antigen or agent, destroy the antigen or agent, and/or develop an immunological memory of the antigen or agent. In some embodiments, the therapeutic product is an antigen or agent which can induce and/or strengthen vaccine-induced memory and/or enable the immune system to respond rapidly and effectively to the antigen or agent in later encounters.

In some embodiments, the therapeutic product is derived from an infectious agent or a part, component, and/or product thereof (e.g., cell wall, genomic sequence, membrane, capsid, protein, lipid, glycan, toxin). In some embodiments, the infectious agent is selected from a virus, a bacterium, a fungus, a protozoan, and a helminth. In some embodiments, the infectious agent is selected a virus and a bacterium.

In some embodiments, the infectious agent is a virus selected from the group consisting of adenovirus; Herpes simplex, type 1; Herpes simplex, type 2; encephalitis virus, papillomavirus; Varicella-zoster virus; Epstein-Barr virus; human cytomegalovirus; human herpes virus, type 8; BK virus; JC virus; Smallpox; polio virus; human bocavirus; Parvovirus B19; human astrovirus; Norwalk virus; coxsackievirus; hepatitis A virus; hepatitis B virus; hepatitis C virus; hepatitis D virus; hepatitis E virus; rhinovirus; severe acute respiratory syndrome (SARS) virus; Yellow Fever virus; Dengue virus; West Nile virus; Rubella virus; Human Immunodeficiency virus (HIV); influenza virus; Guanarito virus; Junin virus; Lassa virus; Machupo virus; Sabia virus; Crimean-Congo hemorrhagic fever virus; Ebola virus; Marburg virus; measles virus; mumps virus; parainfluenza virus; respiratory syncytial virus (RSV); human metapneumovirus; Hendra virus; Nipah virus; rabies virus; Rotavirus; Orbivirus; Coltivirus; Banna virus; human Enterovirus; Hanta virus; West Nile virus; corona virus, SARS-associated coronavirus (SARS-CoV), SARS-CoV-2 virus (COVID-19 associated); Middle East respiratory syndrome corona virus; Japanese encephalitis virus; vesicular exanthernavirus; and eastern equine encephalitis.

In some embodiments, the infectious agent is a bacterium selected from tuberculosis (Mycobacterium tuberculosis), clindamycin-resistant Clostridium difficile, fluoroquinolon-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant Acinetobacter baumannii, and vancomycin-resistant Staphylococcus aureus (VRSA).

In some embodiments, the infectious agent is associated with humans, non-human primates, or other animals, such as birds, pigs, horses, dogs, cats, rabbits, mice, rats, cows, sheep, goats, and deer.

As would be appreciated by a skilled artisan, the terms “antibody therapy” or “antibody-based therapy” are used interchangeably herein to refer to methods of treating a disease or disorder in a subject, wherein the methods comprise the administration of one or more antibodies.

In some embodiments, the RNA polynucleotide described herein comprises an expression sequence encoding a therapeutic product. In some embodiments, the therapeutic product is a polypeptide or protein. In some embodiments, the polypeptide or protein is a therapeutic antibody. In some embodiments, the RNA polynucleotide and compositions comprising the RNA polynucleotide described herein can be used in the manufacturing process of an antibody for use in antibody therapy.

Exemplary therapeutic antibodies include antibodies used for the treatment of cancer. Non limiting examples of therapeutic antibodies include 131I-tositumomab (Follicular lymphoma, B cell lymphomas, leukemias), 3F8 (Neuroblastoma), 8H9, Abagovomab (Ovarian cancer), Adecatumumab (Prostate and breast cancer), Afutuzumab (Lymphoma), Alacizumab pegol, Alemtuzumab (B-cell chronic lymphocytic leukaemia, T-cell-Lymphoma), Amatuximab, AME-133v (Follicular lymphoma, cancer), AMG 102 (Advanced Renal Cell Carcinoma), Anatumomab mafenatox (Non-small cell lung carcinoma), Apolizumab (Solid Tumors, Leukemia, Non-Hodgkin-Lymphoma, Lymphoma), Bavituximab (Cancer, viral infections), Bectumomab (Non-Hodgkin's lymphoma), Belimumab (Non-Hodgkin lymphoma), Bevacizumab (Colon Cancer, Breast Cancer, Brain and Central Nervous System Tumors, Lung Cancer, Hepatocellular Carcinoma, Kidney Cancer, Breast Cancer, Pancreatic Cancer, Bladder Cancer, Sarcoma, Melanoma, Esophageal Cancer; Stomach Cancer, Metastatic Renal Cell Carcinoma; Kidney Cancer, Glioblastoma, Liver Cancer, Proliferative Diabetic Retinopathy, Macular Degeneration), Bivatuzumab mertansine (Squamous cell carcinoma), Blinatumomab, Brentuximab vedotin (Hematologic cancers), Cantuzumab (Colon Cancer, Gastric Cancer, Pancreatic Cancer, NSCLC), Cantuzumab mertansine (Colorectal cancer), Cantuzumab ravtansine (Cancers), Capromab pendetide (Prostate cancer), Carlumab, Catumaxomab (Ovarian Cancer, Fallopian Tube Neoplasms, Peritoneal Neoplasms), Cetuximab (Metastatic colorectal cancer and head and neck cancer), Citatuzumab bogatox (Ovarian cancer and other solid tumors), Cixutumumab (Solid tumors), Clivatuzumab tetraxetan (Pancreatic cancer), CNTO 328 (B-Cell Non-Hodgkin's Lymphoma, Multiple Myeloma, Castleman's Disease, ovarian cancer), CNTO 95 (Melanoma), Conatumumab, Dacetuzumab (Hematologic cancers), Dalotuzumab, Denosumab (Myeloma, Giant Cell Tumor of Bone, Breast Cancer, Prostate Cancer, Osteoporosis), Detumomab (Lymphoma), Drozitumab, Ecromeximab (Malignant melanoma), Edrecolomab (Colorectal carcinoma), Elotuzumab (Multiple myeloma), Elsilimomab, Enavatuzumab, Ensituximab, Epratuzumab (Autoimmune diseases, Systemic Lupus Erythematosus, Non-Hodgkin-Lymphoma, Leukemia), Ertumaxomab (Breast cancer), Ertumaxomab (Breast Cancer), Etaracizumab (Melanoma, prostate cancer, ovarian cancer), Farletuzumab (Ovarian cancer), FBTA05 (Chronic lymphocytic leukaemia), Ficlatuzumab (Cancer), Figitumumab (Adrenocortical carcinoma, non-small cell lung carcinoma), Flanvotumab (Melanoma), Galiximab (B-cell lymphoma), Galiximab (Non-Hodgkin-Lymphoma), Ganitumab, GC1008 (Advanced Renal Cell Carcinoma; Malignant Melanoma, Pulmonary Fibrosis), Gemtuzumab (Leukemia), Gemtuzumab ozogamicin (Acute myelogenous leukemia), Girentuximab (Clear cell renal cell carcinoma), Glembatumumab vedotin (Melanoma, breast cancer), GS6624 (Idiopathic pulmonary fibrosis and solid tumors), HuC242-DM4 (Colon Cancer, Gastric Cancer, Pancreatic Cancer), HuHMFG1 (Breast Cancer), HuN901-DM1 (Myeloma), Ibritumomab (Relapsed or refractory low-grade, follicular, or transformed B-cell non-Hodgkin's lymphoma (NHL)), Icrucumab, ID09C3 (Non-Hodgkin-Lymphoma), Indatuximab ravtansine, Inotuzumab ozogamicin, Intetumumab (Solid tumors (Prostate cancer, melanoma)), Ipilimumab (Sarcoma, Melanoma, Lung cancer, Ovarian Cancer leucemia, Lymphoma, Brain and Central Nervous System Tumors, Testicular Cancer, Prostate Cancer, Pancreatic Cancer, Breast Cancer), Iratumumab (Hodgkin's lymphoma), Labetuzumab (Colorectal cancer), Lexatumumab, Lintuzumab, Lorvotuzumab mertansine, Lucatumumab (Multiple myeloma, non-Hodgkin's lymphoma, Hodgkin's lymphoma), Lumiliximab (Chronic lymphocytic leukemia), Mapatumumab (Colon Cancer, Myeloma), Matuzumab (Lung Cancer, Cervical Cancer, Esophageal Cancer), MDX-060 (Hodgkin-Lymphoma, Lymphoma), MEDI 522 (Solid Tumors, Leukemia, Lymphoma, Small Intestine Cancer, Melanoma), Mitumomab (Small cell lung carcinoma), Mogamulizumab, MORab-003 (Ovarian Cancer, Fallopian Tube Cancer, Peritoneal Cancer), MORab-009 (Pancreatic Cancer, Mesothelioma, Ovarian Cancer, Non-Small Cell Lung Cancer, Fallopian Tube Cancer, Peritoneal Cavity Cancer), Moxetumomab pasudotox, MT103 (Non-Hodgkin-Lymphoma), Nacolomab tafenatox (Colorectal cancer), Naptumomab estafenatox (Non-small cell lung carcinoma, renal cell carcinoma), Narnatumab, Necitumumab (Non-small cell lung carcinoma), Nimotuzumab (Squamous cell carcinoma, head and neck cancer, nasopharyngeal cancer, glioma), Nimotuzumab (Squamous cell carcinomas, Glioma, Solid Tumors, Lung Cancer), Olaratumab, Onartuzumab (Cancer), Oportuzumab monatox, Oregovomab (Ovarian cancer), Oregovomab (Ovarian Cancer, Fallopian Tube Cancer, Peritoneal Cavity Cancer), PAM4 (Pancreatic Cancer), Panitumumab (Colon Cancer, Lung Cancer, Breast Cancer; Bladder Cancer; Ovarian Cancer), Patritumab, Pemtumomab, Pertuzumab (Breast Cancer, Ovarian Cancer, Lung Cancer, Prostate Cancer), Pritumumab (Brain cancer), Racotumomab, Radretumab, Ramucirumab (Solid tumors), Rilotumumab (Solid tumors), Rituximab (Urticaria, Rheumatoid Arthritis, Ulcerative Colitis, Chronic Focal Encephalitis, Non-Hodgkin-Lymphoma, Lymphoma, Chronic Lymphocytic Leukemia), Robatumumab, Samalizumab, SGN-30 (Hodgkin-Lymphoma, Lymphoma), SGN-40 (Non-Hodgkin-Lymphoma, Myeloma, Leukemia, Chronic Lymphocytic Leukemia), Sibrotuzumab, Siltuximab, Tabalumab (B-cell cancers), Tacatuzumab tetraxetan, Taplitumomab paptox, Tenatumomab, Teprotumumab (Hematologic tumors), TGN1412 (Chronic lymphocytic leukemia, rheumatoid arthritis), Ticilimumab (=tremelimumab), Tigatuzumab, TNX-650 (Hodgkin's lymphoma), Tositumomab (Follicular lymphoma, B cell lymphomas, Leukemias, Myeloma), Trastuzumab (Breast Cancer, Endometrial Cancer, Solid Tumors), TRBS07 (Melanoma), Tremelimumab, TRU-016 (Chronic lymphocytic leukemia), TRU-016 (Non-Hodgkin lymphoma), Tucotuzumab celmoleukin, Ublituximab, Urelumab, Veltuzumab (Non-Hodgkin's lymphoma), Veltuzumab (IMMU-106) (Non-Hodgkin's lymphoma), Volociximab (Renal Cell Carcinoma, Pancreatic Cancer, Melanoma), Votumumab (Colorectal tumors), WX-G250 (Renal Cell Carcinoma), Zalutumumab (Head and Neck Cancer, Squamous Cell Cancer), and Zanolimumab (T-Cell-Lymphoma).

Exemplary therapeutic antibodies include antibodies used for the treatment of immune disorders. Non limiting examples of therapeutic antibodies include Efalizumab (Psoriasis), Epratuzumab (Autoimmune diseases, Systemic Lupus Erythematosus, Non-Hodgkin-Lymphoma, Leukemia), Etrolizumab (inflammatory bowel disease), Fontolizumab (Crohn's disease), Ixekizumab (autoimmune diseases), Mepolizumab (Hypereosinophilie-Syndrom, Asthma, Eosinophilic Gastroenteritis, Churg-Strauss Syndrome, Eosinophilic Esophagitis), Milatuzumab (multiple myeloma and other hematological malignancies), Pooled immunoglobulins (Primary immunodeficiencies), Priliximab (Crohn's disease, multiple sclerosis), Rituximab (Urticaria, Rheumatoid Arthritis, Ulcerative Colitis, Chronic Focal Encephalitis, Non-Hodgkin-Lymphoma, Lymphoma, Chronic Lymphocytic Leukemia), Rontalizumab (systemic lupus erythematosus), Ruplizumab (rheumatic diseases), Sarilumab (rheumatoid arthritis, ankylosing spondylitis), Vedolizumab (Crohn's disease, ulcerative colitis), Visilizumab (Crohn's disease, ulcerative colitis), Reslizumab (inflammations of the airways, skin and gastrointestinal tract), Adalimumab (Rheumatoid arthritis, Crohn's disease, Ankylosing spondylitis, Psoriatic arthritis), Aselizumab (severely injured patients), Atinumab (treatment of neurologic systems), Atlizumab (rheumatoid arthritis, systemic juvenile idiopathic arthritis), Bertilimumab (severe allergic disorders), Besilesomab (inflammatory lesions and metastases), BMS-945429, ALD518 (cancer and rheumatoid arthritis), Briakinumab (psoriasis, rheumatoid arthritis, inflammatory bowel diseases, multiple sclerosis), Brodalumab (inflammatory diseases), Canakinumab (rheumatoid arthritis), Canakinumab (cryopyrin-associated periodic syndromes (CAPS), rheumatoid arthritis, chronic obstructive pulmonary disease), Certolizumab pegol (Crohn's disease), Erlizumab (heart attack, stroke, traumatic shock), Fezakinumab (rheumatoid arthritis, psoriasis), Golimumab (rheumatoid arthritis, psoriatic arthritis, ankylosing spondylitis), Gomiliximab (allergic asthma), Infliximab (Rheumatoid arthritis, Crohn's disease, ankylosing spondylitis, psoriatic arthritis, plaque psoriasis, Morbus Bechterew, Colitis ulcerosa), Mavrilimumab (rheumatoid arthritis), Natalizumab (Multiple sclerosis), Ocrelizumab (multiple sclerosis, rheumatoid arthritis, lupus erythematosus, hematological cancer), Odulimomab (prevention of organ transplant rejections, immunological diseases), Ofatumumab (Chronic lymphocytic leukemia, follicular non-Hodgkin's lymphoma, B cell lymphoma, rheumatoid arthritis, relapsing remitting multiple sclerosis, Lymphoma, B-Cell Chronic Lymphocytic Leukemia), Ozoralizumab (inflammation), Pexelizumab (reduction of side effects of cardiac surgery), Rovelizumab (haemorrhagic shock), SBI-087 (Rheumatoid arthritis), SBI-087 (Systemic lupus erythematosus), Secukinumab (uveitis, rheumatoid arthritis psoriasis), Sirukumab (rheumatoid arthritis), Talizumab (allergic reaction), Tocilizumab (rheumatoid arthritis, systemic juvenile idiopathic arthritis, Castleman's disease), Toralizumab (rheumatoid arthritis, lupus nephritis), TRU-015 (Rheumatoid arthritis), TRU-016 (Autoimmune disease and inflammation), Ustekinumab (multiple sclerosis, psoriasis, psoriatic arthritis), Ustekinumab (IL-12/IL-23 blocker) (Plaque-Psoriasis, psoriatic arthritis, multiple sclerosis, sarcoidosis, the latter versus), Vepalimomab (inflammation), Zolimomab aritox (systemic lupus erythematosus, graft-versus-host disease), Sifalimumab (SLE, dermatomyositis, polymyositis), Lumiliximab (Allergies), and Rho(D) Immune Globulin (Rhesus disease); or are selected from antibodies used for the treatment of infectious diseases, e.g. Afelimomab (sepsis), CR6261 (infectious disease/influenza A), Edobacomab (sepsis caused by gram-negative bacteria), Efungumab (invasive Candida infection), Exbivirumab (hepatitis B), Felvizumab (respiratory syncytial virus infection), Foravirumab (rabies (prophylaxis)), Ibalizumab (HIV infection), Libivirumab (hepatitis B), Motavizumab (respiratory syncytial virus (prevention)), Nebacumab (sepsis), Tuvirumab (chronic hepatitis B), Urtoxazumab (diarrhoea caused by E. coli), Bavituximab (diverse viral infections), Pagibaximab (sepsis (e.g. Staphylococcus)), Palivizumab (prevention of respiratory syncytial virus infection in high-risk paediatric patients), Panobacumab (Pseudomonas aeruginosa infection), PRO 140 (HIV infection), Rafivirumab (rabies (prophylaxis)), Raxibacumab (anthrax (prophylaxis and treatment)), Regavirumab (cytomegalovirus infection), Sevirumab (cytomegalovirus infection), Suvizumab (viral infections), and Tefibazumab (Staphylococcus aureus infection).

Exemplary therapeutic antibodies include antibodies used for the treatment of blood disorders. Non limiting examples of therapeutic antibodies include Abciximab (percutaneous coronary intervention), Atorolimumab (hemolytic disease of the newborn), Eculizumab (Paroxysmal nocturnal haemoglobinuria), Mepolizumab (Hypereosinophilie-Syndrom, Asthma, Eosinophilic Gastroenteritis, Churg-Strauss Syndrome, Eosinophilic Esophagitis), and Milatuzumab (multiple myeloma and other hematological malignancies).

Exemplary therapeutic antibodies include antibodies used for immunoregulation. Non-limiting examples of therapeutic antibodies include Antithymocyte globulin (Acute kidney transplant rejection, aplastic anaemia), Basiliximab (Prophylaxis against allograft rejection in renal transplant patients receiving an immunosuppressive regimen including cyclosporine and corticosteroids), Cedelizumab (prevention of organ transplant rejections, treatment of autoimmune diseases), Daclizumab (Prophylaxis against acute allograft rejection in patients receiving renal transplants, Multiple Sclerosis), Gavilimomab (graft versus host disease), Inolimomab (graft versus host disease), Muromonab-CD3 (prevention of organ transplant rejections), Muromonab-CD3 (Acute renal allograft rejection or steroid-resistant cardiac or hepatic allograft rejection), Odulimomab (prevention of organ transplant rejections, immunological diseases), and Siplizumab (psoriasis, graft-versus-host disease (prevention)).

Exemplary therapeutic antibodies include antibodies used for treatment of diabetes, Alzheimer's disease or asthma. Non-limiting examples of therapeutic antibodies include Gevokizumab (diabetes), Otelixizumab (diabetes mellitus type 1), and Teplizumab (diabetes mellitus type 1); Bapineuzumab, Crenezumab, Gantenerumab, Ponezumab, R1450, and Solanezumab (Alzheimer's disease); Benralizumab, Enokizumab, Keliximab, Lebrikizumab, Omalizumab, Oxelumab, Pascolizumab, and Tralokinumab (asthma).

### 8.7.4. Cell Therapy

As would be appreciated a skilled artisan, the term “cell therapy” or “cellular therapy” are used interchangeably herein to refer to methods of treating a disease, disorder and/or injury in a subject wherein the method comprises the administration of one or more cells (i.e. a plurality of cells) to the subject.

Non-limiting examples of diseases, disorder and/or injuries that can be treated with cell therapy include cancer (both hematological cancers and solid tumors), compromised bone marrow (bone marrow that has been comprised and/or damaged due to disease, infection, radiation and/or chemotherapy), infection, spinal cord injuries, diabetes, spinal cord injuries, type 1 diabetes, Parkinson's disease, amyotrophic lateral sclerosis, Alzheimer's disease, heart disease, stroke, burns, osteoarthritis, autoimmune diseases, infectious diseases, amyloidosis, acute leukemia, acute myeloid leukemia, Amegakaryocytosis or congenital thrombocytopenia, Aplastic anemia or refractory anemia, chronic lymphocytic leukemia, familial erythrophagocytic lymphohistiocytosis, myelodysplastic syndrome of another myelodysplastic disorder, osteopetrosis, paroxysmal nocturnal hemoglobinuria, Wiskott-Aldrich syndrome, Hodgkin lymphoma, non-Hodgkin lymphoma, multiple myeloma and tissue/organ damage.

As would be appreciated by a skilled artisan, cell therapy encompasses allogeneic cell therapies (wherein the cells that are transplanted into the subject are derived from a human donor that is not the subject), autologous cell therapies (wherein the cells that are transplanted into the subject are derived from the subject) and xenogeneic cell therapies (wherein the cells that are transplanted are derived from a species other than human).

As would be appreciated by a skilled artisan, cell therapy can encompass the administration of several different cell types, including, but not limited to, blood cells, embryonic stem cells, neural stem cells, mesenchymal stem cells, hematopoietic stem cells, cadiomyocytes, islet cells, chondrocytes, T cells, tumor-infiltrating lymphocytes, engineered T cells, Natural Killer (NK) cells, chimeric antigen receptor (CAR)-T cells, red blood cells, white blood cells, platelets, pluripotent stem cells, fibroblasts, keratinocytes, hepatocytes, dendritic cells, macrophages or any combination thereof.

In some embodiments, the cell therapy is a CAR-T cell therapy. Non-limiting examples of an autologous CAR-T cell based therapy include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, and AUTO4 from Autolus Limited. Non-limiting examples of an allogeneic CAR-T cell based therapy include UCARTCS from Cellectis, PBCAR19B and PBCAR269A from Precision Biosciences, FT819 from Fate Therapeutics, and CYAD-211 from Clyad Oncology.

In some embodiments, the cell therapy is a dendritic cell (DC) vaccine therapy. The term “dendritic cell vaccine”, as used herein, refers to a vaccine comprising dendritic cells which are loaded with the antigens against which an immune reaction is desired.

As would be appreciated by the skilled artisan, the term “dendritic cell (DC)” refers to an antigen presenting cell capable of sensitizing HLA-restricted T cells. DCs include DCs derived from bone marrow hematopoietic cells such as plasmacytoid dendritic cells, myeloid dendritic cells, Langerhans cells and interdigitating cells; and follicular DCs. Dendritic cells may be recognized by function, or by phenotype, particularly by cell surface phenotype. These cells are characterized by their distinctive morphology having veil-like projections on the cell surface, intermediate to high levels of surface HLA-class II expression and ability to present antigen to T cells, particularly to naive T cells (See Steinman R, et al., Ann. Rev. Immunol. 1991; 9:271-196). Typically, cell surface phenotype of DCs include CD1a+, CD4+, CD86+, or HLA-DR. The term DCs encompasses both immature and mature DCs.

Typically, in the generation of an anti-cancer DC-based vaccine, DCs are expanded ex vivo and contacted with a cancer antigen or a cancer cell lysate to thereby induce presentation of the cancer antigen (see e.g. Nestle, F. et al. (1998) Nature Medicine 4: 328-332). Alternatively, promoting presentation of a cancer antigen by a DC comprises transfecting the DC with a DNA, cDNA, an mRNA a RNA polynucleotide described herein or any combination thereof, that encodes for a cancer antigen. Non-limiting examples of cancer antigens include MAGE-AI, MAGE-A2, MAGE-A3, MAGE-A4, MAGE-AS, MAGE-A6, MAGE-A7, MAGE-AS, MAGE-A9, MAGE-AIO, MAGE-A1 1, MAGE-A12, GAGE-I, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-1, LAGE-1, SSX-1, SSX-2(HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gplOO, gp75, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferase AS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, plSOerbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, 0250, Ga733 (EpCAM), HTgp-175, M344, MA-50, MG7-Ag, MOV18, NB\170K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2 and tumor derived heat shock proteins.

As would be appreciated by the skilled artisan, pluralities of cells that are administered as part of a cell therapy can comprise a single cell type or two or more different cell types. Cell therapy that comprises the administration of two or more cell types can be referred to as “multicellular therapy”.

As would be appreciated by the skilled artisan, cells that are transplanted into a subject as part of a cell therapy can be modified or unmodified. Accordingly, in some embodiments, the nucleic acid molecules and compositions comprising nucleic acid molecules described herein can be used in the modification of cells that are to be transplanted into a subject as part of a cell therapy. In a non-limiting example, the nucleic acid molecules of the present disclosure can be introduced into the cells that are to be transplanted as part of a cell therapy using standard methods known in the art or methods described herein. In some embodiments, the nucleic acid molecules and compositions comprising nucleic acid molecules described herein can be used in the manufacturing process of the cell therapy.

### 8.8. Articles of Manufacture or Kits

An article of manufacture or a kit is provided comprising RNA polynucleotides is also provided herein. The article of manufacture or kit can further comprise a package insert comprising instructions for using the genetically engineered immune cells to treat or delay progression of cancer in an individual or to enhance immune function of an individual having cancer. Any of the RNA polynucleotides described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### 8.9. Assays

Various IRES-like sequences, endogenous IRES sequences or variants thereof, or combinations thereof, may be tested for their ability to attracts a eukaryotic ribosomal translation initiation complex and/or promote translation initiation. The assays below are described for IRES-like sequences but can be performed analogously for endogenous IRES sequences or variants thereof, combinations of IRES-like sequences and endogenous IRES sequences or variants thereof, combinations of one or more IRES-like sequences, or combinations of endogenous IRES sequences or variants thereof.

### 8.9.1. Reference Value

A reference value is useful to select an IRES-like sequence, endogenous IRES sequence or a variant thereof, or a combination thereof that is able to attract a eukaryotic ribosomal translation initiation complex and/or promote translation initiation, as compared to sequences which are minimally able to, or are unable to or are considered unable to, attract a eukaryotic ribosomal translation initiation complex and/or promote translation initiation. In some embodiments, the reference value is characteristic of the absence of the target therapeutic product expression. In some embodiments, the reference value is the mean of the output value of the total input population, e.g., the mean of the expression level of the therapeutic product, of all the input IRES-like sequences, endogenous IRES sequences or variants thereof, or combinations thereof. In some embodiments, the reference value is the average output value of the total input population, e.g., the average of the expression level of the therapeutic product, of all the input IRES-like sequences, endogenous IRES sequences or variants thereof, or combinations thereof. In some embodiment, the reference value is about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% of the highest output value of the total input population, e.g., the highest expression level of the therapeutic product, of all the input IRES-like sequences, endogenous IRES sequences or variants thereof, or combinations thereof. In some embodiments, the reference value is the average output value of the total input population, e.g., the average of the expression level of the therapeutic product, of all the input IRES-like sequences, endogenous IRES sequences or variants thereof, or combinations thereof. In some embodiment, the reference value is about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.10% of the highest output value of the total input population, e.g., the highest expression level of the therapeutic product, of all the input IRES-like sequences, endogenous IRES sequences or variants thereof, or combinations thereof. In some embodiment, the reference value is at least about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% higher than the lowest output value of the total input population, e.g., the lowest expression level of the therapeutic product, of all the input IRES-like sequences, endogenous IRES sequences or variants thereof, or combinations thereof. In some embodiments, the reference value is the average output value of the total input population, e.g., the average of the expression level of the therapeutic product, of all the input IRES-like sequences, endogenous IRES sequences or variants thereof, or combinations thereof. In some embodiment, the reference value is at least about 95%, about 90%, about 85%, about 80%, about 75%, about 70%, about 65%, about 60%, about 55%, or about 50% higher the lowest output value of the total input population, e.g., the lowest expression level of the therapeutic product, of all the input IRES-like sequences, endogenous IRES sequences or variants thereof, or combinations thereof. In some embodiment, the reference value is greater than or equal to 0. In some embodiments, the reference value is greater than 0. In some embodiment, the reference value is at least about 99.995% higher the lowest output value of the total input population, e.g., the lowest expression level of the therapeutic product, of all the input IRES-like sequences, endogenous IRES sequences or variants thereof, or combinations thereof. In some embodiment, the reference value is at least about 99.999%, about 99.998%, about 99.997%, about 99.996% or about 99.995% higher the lowest output value of the total input population, e.g., the lowest expression level of the therapeutic product, of all the input IRES-like sequences, endogenous IRES sequences or variants thereof, or combinations thereof. In some embodiment, the reference value is at least about 99.9999%, about 99.9998%, about 99.9997%, about 99.9996% or about 99.9995% higher the lowest output value of the total input population, e.g., the lowest expression level of the therapeutic product, of all the input IRES-like sequences, endogenous IRES sequences or variants thereof, or combinations thereof. In some embodiment, the reference value is at least about 99.99999%, about 99.99998%, about 99.99997%, about 99.99996% or about 99.99995% higher the lowest output value of the total input population, e.g., the lowest expression level of the therapeutic product, of all the input IRES-like sequences, endogenous IRES sequences or variants thereof, or combinations thereof. In some embodiment, the reference value is at least about 99.999999%, about 99.999998%, about 99.999997%, about 99.999996% or about 99.999995% higher the lowest output value of the total input population, e.g., the lowest expression level of the therapeutic product, of all the input IRES-like sequences, endogenous IRES sequences or variants thereof, or combinations thereof. In some embodiment, the reference value is at least about 99.9999999%, about 99.9999998%, about 99.9999997%, about 99.9999996% or about 99.9999995% higher the lowest output value of the total input population, e.g., the lowest expression level of the therapeutic product, of all the input IRES-like sequences, endogenous IRES sequences or variants thereof, or combinations thereof.

### 8.9.2. Assessing Therapeutic Product Expression Levels and/or Activities

The level of a therapeutic product, such as a polypeptide, a protein, an antibody, or an enzyme, can be determined by any method known in the art or described herein. In some embodiments, the level of a therapeutic product, such as a polypeptide, a protein, an antibody, or an enzyme, can be determined by assessing (e.g., quantifying) transcribed RNA produced from the IRES-like sequences, endogenous IRES sequences or variants thereof, or combinations thereof of the present disclosure in a sample from a subject, by using, e.g., Northern blotting, PCR analysis, real time PCR analysis, or any other technique known in the art or described herein. In some embodiments, the level of a therapeutic product, such as a polypeptide, a protein, an antibody, or an enzyme can be determined by assessing (e.g., quantifying) the mRNA produced from the IRES-like sequences, endogenous IRES sequences or variants thereof, or combinations thereof of the present disclosure in a sample from a subject. In some embodiments, the level of a therapeutic product, such as a polypeptide, a protein, an antibody, or an enzyme, can be determined by assessing (e.g., quantifying) the level of polypeptide or protein expression of the therapeutic product produced from the IRES-like sequences, endogenous IRES sequences or variants thereof, or combinations thereof of the present disclosure in a sample from a subject, by using, e.g., immunohistochemical analysis, Western blotting, ELISA, immunoprecipitation, flow cytometry analysis, or any other technique known in the art or described herein.

In some embodiments, samples may be collected from various tissues or organs of the subject where a therapeutic product is produced or suspected of being produced. In some embodiments, samples may be collected from one or more tissues selected from epithelia tissue (e.g., skin tissue, lining of the GI tract), connective tissue (e.g., blood, fat, bone, tendon), muscle tissue (e.g., cardiac muscle, smooth muscle, skeletal muscle), and nervous tissue (e.g., brain tissue, tissue from the spinal cord, tissue from nerves). In some embodiments, samples may be collected from one or more organs selected from head, neck, brain, eye, ear, nose, tongue, throat, heart, lung, pancreas, spleen, thyroid, esophagus, stomach, intestine, liver, gall bladder, colon, bladder, kidney, breast, ovary, uterus, testicle, cervix, skin, blood, and lymph node and a part thereof. In some embodiments, the subject is any subject described herein. In some embodiments, the subject is a human. In some embodiments, the subject is a human suffering from a disease or disorder, for example, a disease or disorder in which the therapeutic product is involved in initiation, development, and/or manifestation of the disease or disorder.

In some embodiments, samples may be a primary cell, a cell line derived from a primary cell, an immortalized cell, or a mixture thereof.

In some embodiments, the level of a therapeutic product is measured directly, i.e., without a tag or a marker. In some embodiments, the level of a therapeutic product is measured indirectly, i.e., through a tag or a marker. In some embodiments, a therapeutic product is fused with a tag or a marker. Any tag or marker known in the art to measure gene/protein expression may be used. In some embodiments, the tag or marker is selected from a fluorescent protein (e.g., GFP, BFP, YFP, RFP), an enzyme (e.g., luciferase), an HA tag, a His tag, a FLAG tag, a Myc tag, and a combination thereof.

In particular embodiments, the level of the protein is determined by a method capable of quantifying the amount of the therapeutic product present in a tissue sample of a patient (e.g., in human serum), and/or capable of detecting the correction of the level of protein following treatment with the RNA polynucleotide, the polypeptide, the DNA vector, or the cell described herein.

In some embodiments, the level of the therapeutic product in a tissue sample is determined by assessing (e.g., quantifying) protein expression of the therapeutic product in the sample using ELISA, Western blotting, or luciferase assay.

### 8.9.3. Screening Assays

Various IRES-like sequences, endogenous IRES sequences or variants thereof, or combinations thereof, may be tested for their ability to attracts a eukaryotic ribosomal translation initiation complex and/or promote translation initiation. The assays below are described for IRES-like sequences but can be performed analogously for endogenous IRES sequence or a variant thereof, a combination of IRES-like sequence and endogenous IRES sequence or a variant thereof, a sequence comprising one or more IRES-like sequences or endogenous IRES sequences or variants thereof.

### 8.9.3.1. Expression Level

For example, the effect of an IRES-like sequence, endogenous IRES sequence or a variant thereof, or a combination thereof disclosed herein on the expression of level of a therapeutic product may be assessed. In some embodiments, the effect may be assessed through in vitro translation of an RNA polynucleotide or circular RNA comprising an IRES-like sequence, endogenous IRES sequence or a variant thereof, or a combination thereof disclosed herein in a cell free system. In some embodiments, the effect may be assessed through transfection/transformation of a cell with an RNA polynucleotide or circular RNA comprising an IRES-like sequence, endogenous IRES sequence or a variant thereof, or a combination thereof disclosed herein and expression of the RNA polynucleotide or circular RNA. The expression level of the therapeutic product may be assessed according to any method known in the art and/or described herein, e.g., immunohistochemical analysis, Western blotting, ELISA, immunoprecipitation, and flow cytometry analysis.

In some embodiments, IRES-like sequences, endogenous IRES sequences or variants thereof, or combinations thereof identified based on their effect on the expression of level of a therapeutic product (e.g., an expression marker such as a fluorescence protein or a luciferase) may be further assessed for their ability to promote, facilitate, or regulate (e.g., increase or decrease) the therapeutic product expression in vitro. In some embodiments, IRES-like sequences, endogenous IRES sequences or variants thereof, or combinations thereof may be further assessed in animal models for their ability to promote, facilitate, or regulate (e.g., increase or decrease) the therapeutic product expression in vivo. In some embodiments, IRES-like sequences, endogenous IRES sequences or variants thereof, or combinations thereof may be further assessed in animal models for their ability to promote, facilitate, or regulate (e.g., increase or decrease) the therapeutic product expression in a specific tissue or organ.

Non-limiting illustrative examples of the various assays or methods are provided below.

### In Vitro Transfection and Translation

A desirable amount of a circular RNA can be transfected into cells (e.g., prokaryotic cells or eukaryotic cells) using a transfecting agent, such as Lipofectamine 3000 (Invitrogen). In vitro translation of a desirable amount of a circular RNA may be performed in a cell free lysate. The cell lysate may be collected to analyze the protein expression after incubation.

### Western Blot

Cells transfected with an RNA polynucleotide or circular RNA comprising an IRES-like sequence, endogenous IRES sequence or a variant thereof, or a combination thereof of the present disclosure according to the methods or techniques described herein may be lysed. The total cell lysates are resolved, e.g., through electrophoresis, such as with 4-20% ExpressPlus™ PAGE Gel (GeneScript®). The proteins are transferred to a membrane (e.g., PVDF membrane) to be probed with an antibody against the protein. A secondary antibody binding to the first antibody may be used to stain the membrane for detection. Positive controls such as such as known Gtx, Rsv, CrPV, PSIV, or TSV IRES may be used.

### Luciferase Assay

Cells transfected with an RNA polynucleotide or circular RNA comprising an IRES-like sequence, endogenous IRES sequence or a variant thereof, or a combination thereof of the present disclosure according to the methods or techniques described herein may be lysed. A luciferase reporter assay, such as the dual-luciferase reporter assay system from Promega™, is used to generate the luminescent signal. The luminescence is measured, for example, by Bio-Tek synergy H1.

### Flow Cytometry

Cells transfected with an RNA polynucleotide or circular RNA comprising an IRES-like sequence, endogenous IRES sequence or a variant thereof, or a combination thereof of the present disclosure according to the methods or techniques described herein are collected for flow cytometry, for example, by using BD FACSAria II. To select the singlets, SSC-A vs FSC-A may be used to select 293T cells. Two round selections of singlets may be used by SSC-W vs FSC-H and FSC-W vs FSC-H. FITC-A vs FSC-A may be used to select GFP-positive cells, and the expression level may determined by the level of fluorescence.

### ELISA

ELISA may be carried out according to Bull World Health Organ. 54(2):129-39 (1976) (PMID: 798633).

Optionally, the therapeutic product may be derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatable crosslinkers, or any combinations thereof. Optionally, the expression level of the therapeutic product may be assessed by measuring the level of the derivatizing groups.

### 8.9.3.2. Biological Activities

The biological activities or functions of an IRES-like sequence, endogenous IRES sequence or a variant thereof, or a combination thereof disclosed herein may be assessed according to any method known in the art and/or described herein, e.g., in vivo imaging and PET. For example, the biological activity may include inhibition of tumor growth, and may be assessed in animal models, such as cell-line-derived xenograft (CDX) and patient-derived xenograft (PDX) model.

Non-limiting illustrative examples of the various assays or methods are provided below.

### In Vivo Imaging

For detection of in vivo expression, female BALB/c mice aged 6-8 weeks may be used for administration with an RNA polynucleotide or circular RNA comprising an IRES-like sequence, endogenous IRES sequence or a variant thereof, or a combination thereof of the present disclosure (e.g., a luciferase-encoding circular RNA). The administration may be via intramuscular (i.m.), subcutaneous (s.c.), or intranasal (i.n.) routes. At certain times post administration, animals are injected intraperitoneally (i.p.) with luciferase substrate. Fluorescence signals are collected, for example, by IVIS Spectrum instrument (PerkinElmer). For in vitro imaging, tissues including brain, heart, liver, spleen, lung, kidney, and muscle from the animals are collected immediately, and fluorescence signals of each tissue are measured, for example, by IVIS imager. The fluorescence signals in regions of interest (ROIs) are quantified, e.g., by using Living Image 3.0.

### PET

Mouse tumor xenografts are formed with tumor cell lines. PET scans are performed before and after administration of the animals with an RNA polynucleotide or circular RNA comprising an IRES-like sequence, endogenous IRES sequence or a variant thereof, or a combination thereof of the present disclosure. For example, PET scans may be performed 1 h after a 3.7- to 7.4-MBq administration. A second PET scan may be performed at suitable time points after further administrations.

For example, the assay can be used to access the competitive binding ability of the expressed therapeutic product in a biological system.

For example, the assay can be used to access the enzymatic ability of the expressed therapeutic product in a biological system.

For example, the assay can be used to access the inhibition activities of the expressed therapeutic product in a biological system.

Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the therapeutic product on the molecular target.

### 8.9.4. Secondary Structure Analysis

Stem-loop structure is a type of an RNA secondary structure, which can be determined by any suitable polynucleotide folding algorithm. The energy associated with the secondary structure of a nucleic acid (e.g., an RNA) is free energy which incorporates both enthalpy and entropic contributions. The size of helix and different base-pairing techniques are used to minimize the free energy of RNA secondary structure. This energy is referred to as Gibbs free energy, or AG (kcal/mol). Different techniques have been proposed, but “minimum free energy” is most widely used to predict the RNA secondary structure. Specifically, the structure having lowest Gibbs free energy provides more stability to the secondary structure and the stabilization of RNA molecules and the minimization of free energy can be obtained by stacking of base pairs in a helix. Loops and budges put the negative effect on the stability of the structure. In general, stability and accuracy of the secondary structure of a nucleic acid (e.g., an RNA) are measured based on the amount of free energy releases while forming the correct base pairs. As the negativity in free energy released by a structure increases, it gives a more stable sequence of base pairs. (Zuker M (1994) Prediction of RNS secondary structure by energy minimization, Volume 25 of COMPUTER ANALYSIS OF SEQUENCE DATA: PART II, Grifn A M, Grifn H G (eds), Chapter 23, CRC Press, Inc., Totowa, NJ, pp 267-294.) Various algorithms have been developed to calculate the MFE value of a nucleic acid, which can be either a global algorithm or a local (sliding window) algorithm. In some embodiments, the MFE value of a nucleic acid (e.g., an RNA) is computed using a global algorithm. In some embodiments, the MFE value of a nucleic acid (e.g., an RNA) is computed using a local (sliding window) algorithm.

Among the various algorithms available in the art, Dynamic programming (DP) is as an approach that can be used to compute the MFE value of RNA molecules in the absence of pseudoknots. The benchmarking of RNA folding algorithm named mFold is dependent on DP. (Zuker and Stiegler, Nucleic Acids Res. 9 (1981), 133-148.) Nature inspired optimization algorithms such as evolutionary algorithms (EAs), genetic algorithms (GAs) can also be used. Particle swarm optimization (PSO) techniques have also been adopted. Exemplary available algorithms for MFE value calculation include, for example, RnaPredict, RNAfold, HSRNAFold, SetPSO, HelixPSO, SARNAPredict, ACRNA, PSOfold, efold, and EAMD. RNAfold is an online folding algorithm developed by the Institute for Theoretical Chemistry at the University of Vienna using a centroid structure prediction algorithm (e.g., AR Gruber et al., 2008, Cell 106 (1): 23-24; and PA Carr and GM Church, 2009, Nature Biotechnology 27 (12): 1151-62). RnaPredict applies selection, recombination, and mutation operators on to file the minimal energy structure. HSRNAFold algorithm uses the musical techniques for better searching results. SetPSO is based on PSO algorithm to solve the energy minimization problem in RNA. The HelixPSO algorithm works similar to RNAPredict algorithm and takes set of helices to obtain a secondary structure from RNA sequence. SARNAPredict is also a permutation-based algorithm like HelixPSO, which uses the method of cooling a substance of Simulated Annealing algorithm with a simple thermodynamic model. ACRNA uses ant colony optimizer and PSOfold are both PSO based algorithms. Additionally, environmental adaption method for dynamic environment (EAMD) is a meta-heuristic optimization algorithm. (e.g., Wiese and Glen (2003) Biosystems, 72(1):29-41; Clote (2005) J Comput Biol 12(1):83-101; Geem et al. (2001) Simulation 76(2):60-68; Gruber et al., 2008, Cell 106 (1): 23-24; Carr and Church, 2009, Nature Biotechnology 27(12): 1151-62; Mohsen et al (2010) An optimization algorithm based on harmony search for RNA secondary structure prediction. In: Geem Z W (eds) RECENT ADVANCES IN HARMONY SEARCH ALGORITHM. STUDIES IN COMPUTATIONAL INTELLIGENCE, vol 270. Springer, Berlin, Heidelberg; Geis and Middendorf (2011) Int J Intell Comput Cybern 4(2):160-186; Liu et al. (2011) Chem Res Chin Univ 27(1):108-112; Yu et al. (2010) J Bionic Eng 7(4):382-389; Wu et al. (2011) A fuzzy adaptive particle swarm optimization for “RNA secondary structure” prediction. In: Information science and technology (ICIST), 2011 international conference on 2011 Mar. 26, IEEE, pp 1390-1393.) Additional algorithms can be found in U.S. Provisional Application No. 61/836,080, which is incorporated herein by reference.

In some embodiments, whether an RNA can function as a group II intron is determined using an online predicting tool or a predicting software. An example of such online predicting tool is the online web server “http://webapps2.ucalgary.ca/” created by Zimmerly lab, University of Calgary.

### 8.9.5. In Vitro Self-Splicing Assay

Various in vitro assays are available in the art to test and confirm the self-splicing activity of a given cRNAzyme. The procedure below is described for illustrative purposes. The DNA sequence encoding a subject cRNAzyme is into a proper vector, e.g., pUC57. Vectors are amplified and purified, and linearized using BamHI for in vitro transcription. Radiolabeled transcripts are prepared using T7 RNA polymerase, 5 mM MgCl2, 40 mM Tris-HCl pH 7.5, 0.05% Triton X-100, 10 pCi [α-31P]UTP (3000 Ci mmol−1), 0.5 mM UTP, and 1 mM other NTPs. In vitro transcription reactions are done for 1 h at 37° C. followed by purification of the RNA product on a denaturing 4% (19:1) polyacrylamide, 8M urea, 1×TBE gel. Radiolabeled RNA product is refolded in 40 mM Tris-HCl pH 7.5 through heating at 90° C. for 1 min followed by incubation in 40 mM Tris-HCl pH 7.5, 10 mM MgCl2 for 15 min. To initiate self-splicing, RNA was mixed with an equal volume of 2X splicing buffer (2M NH4Cl, 40 mM Tris-HCl pH 7.5). Reactions are quenched by mixing with an equal volume of 80% formamide, 100 mM EDTA. Splicing products are resolved using denaturing 4% (19:1) polyacrylamide, 8M urea, 1x TBE gels. All splicing assays are done in triplicate.

The self-splicing products can be quantified using any methods known in the art. For example, splicing gels are exposed to storage phosphor screens and splicing products are quantitated using Quantity Analysis Software. Unequal loading can be accounted for by normalizing to an internal control RNA. Band intensity is determined by dividing the background subtracted intensity of each band by the number of uridine residues in the RNA sequence corresponding to the band. All band intensities are then normalized to an unspliced control to give fractional values of input RNA.

### 8.9.6. Electron Microscopy (EM)

EM is an effective tool to visualize the structure of a cRNAzyme. An exemplary EM procedure is provided below to determine the structure of a group 11 intron.

For negative staining EM, 4 μl of the solution containing the cRNAzyme is applied to a glow-discharged holey carbon grid that is pre-coated with a thin layer of continuous carbon film over the holes. After 1 min, the grid is washed consecutively with three droplets of 2% (w/v) uranyl acetate solution for total of 30 sec. After another 1 min, the residual stain is blotted off and the grid was air-dried. The EM data for the negatively-stained specimen is collected on an FEI Tecnai-12 transmission electron microscope, equipped with a LaB6 filament and operated at 120 kV acceleration voltage. Images are collected at a nominal magnification of 68,000×, using a total dose of about 20 e−/Å2 and defocus value ranging from −1.0 to −1.5 μm on a Gatan Ultrascan4000 CCD camera, with a pixel size of 1.65 Å on the object scale. Total of 66 tilt-pair images of the specimen at 0° and 500 are recorded manually for the random-conical tilt 3D reconstruction.

For cryo-EM, frozen-hydrated specimens are prepared using the FEI Vitrobot Mark IV plunger. 4 μl of the diluted sample is placed on a glow-discharged holey carbon grid (Quantifoil Cu-Rh R1.2/1.3) pre-coated with continuous carbon film (with −2 nm thickness). The excess of solution from the grid is blotted for 2.0 s at 100% humidity at 22° C. before the grid is flash frozen into liquid ethane slush cooled at liquid nitrogen temperature. Cryo-EM data are collected on an FEI Titan Krios electron microscope, equipped with a Gatan K2 Summit direct-electron counting camera. The microscope is operated at 300 kV and images of the specimen are recorded with a defocus range of −1.2 to −3 μm at a calibrated magnification in super resolution mode of the K2 camera, yielding a pixel size of about 0.6 Å on the object scale. 1000 to 5000 movie stacks, each containing 32 sub-frames, are recorded using the semi-automated low-dose acquisition program UCSF-Image4, with an electron dose rate of 6.25 electrons per A2 per second and total exposure time of 8 seconds.

### 8.10. Sequences

Table 1. Exemplary Pentamer (5-mer) Sequences and Enrichment Scores
Tables 2-96. Exemplary IRES-like Sequences and Numerical Scores
Table 97. Exemplary Sequences Comprising IRES-like Sequences and Natural IRES Sequences
Tables 98-100. Exemplary Natural IRES Sequences
Table 101. Exemplary Nucleic Acid Sequences of Target Products
Table 102. Exemplary Amino Acid Sequences of Target Products
Table 103. Exemplary E1 Sequences
Table 104. Exemplary E2 Sequences
Table 105. Exemplary Group II Intron Sequences
Table 106. Exemplary 3′ Intron Fragment Sequences
Table 107. Exemplary 5′ Intron Fragment Sequences
Table 108. Exemplary Self-splicing ribozyme cRNAzyme comprising IRES-like Sequences for Circular Constructs
Table 109. Exemplary 5′ Homology Arm and 3′ Homology Arm Sequences
Table 110. Exemplary IRES-like Sequences and Numerical Scores
Table 111. Exemplary circular RNA for luciferase reporter assay
Table 112. Exemplary IRES-like Sequences and Numerical Scores
Table 113. Foldback primer

Lengthy table referenced here
US20250320519A1-20251016-T00001
Please refer to the end of the specification for access instructions.

Lengthy table referenced here
US20250320519A1-20251016-T00002
Please refer to the end of the specification for access instructions.

Lengthy table referenced here
US20250320519A1-20251016-T00003
Please refer to the end of the specification for access instructions.

Lengthy table referenced here
US20250320519A1-20251016-T00004
Please refer to the end of the specification for access instructions.

Lengthy table referenced here
US20250320519A1-20251016-T00005
Please refer to the end of the specification for access instructions.

Lengthy table referenced here
US20250320519A1-20251016-T00006
Please refer to the end of the specification for access instructions.

Lengthy table referenced here
US20250320519A1-20251016-T00007
Please refer to the end of the specification for access instructions.

Lengthy table referenced here
US20250320519A1-20251016-T00008
Please refer to the end of the specification for access instructions.

Lengthy table referenced here
US20250320519A1-20251016-T00009
Please refer to the end of the specification for access instructions.

Lengthy table referenced here
US20250320519A1-20251016-T00010
Please refer to the end of the specification for access instructions.

Lengthy table referenced here
US20250320519A1-20251016-T00011
Please refer to the end of the specification for access instructions.

Lengthy table referenced here
US20250320519A1-20251016-T00012
Please refer to the end of the specification for access instructions.

Lengthy table referenced here
US20250320519A1-20251016-T00013
Please refer to the end of the specification for access instructions.

Lengthy table referenced here
US20250320519A1-20251016-T00014
Please refer to the end of the specification for access instructions.

Lengthy table referenced here
US20250320519A1-20251016-T00015
Please refer to the end of the specification for access instructions.

Lengthy table referenced here
US20250320519A1-20251016-T00016
Please refer to the end of the specification for access instructions.

Lengthy table referenced here
US20250320519A1-20251016-T00017
Please refer to the end of the specification for access instructions.

Lengthy table referenced here
US20250320519A1-20251016-T00018
Please refer to the end of the specification for access instructions.

Lengthy table referenced here
US20250320519A1-20251016-T00019
Please refer to the end of the specification for access instructions.

Lengthy table referenced here
US20250320519A1-20251016-T00020
Please refer to the end of the specification for access instructions.

Lengthy table referenced here
US20250320519A1-20251016-T00021
Please refer to the end of the specification for access instructions.

Lengthy table referenced here
US20250320519A1-20251016-T00022
Please refer to the end of the specification for access instructions.

Lengthy table referenced here
US20250320519A1-20251016-T00023
Please refer to the end of the specification for access instructions.

Lengthy table referenced here
US20250320519A1-20251016-T00024
Please refer to the end of the specification for access instructions.

Lengthy table referenced here
US20250320519A1-20251016-T00025
Please refer to the end of the specification for access instructions.

Lengthy table referenced here
US20250320519A1-20251016-T00026
Please refer to the end of the specification for access instructions.

Lengthy table referenced here
US20250320519A1-20251016-T00027
Please refer to the end of the specification for access instructions.

Lengthy table referenced here
US20250320519A1-20251016-T00028
Please refer to the end of the specification for access instructions.

Lengthy table referenced here
US20250320519A1-20251016-T00029
Please refer to the end of the specification for access instructions.

Lengthy table referenced here
US20250320519A1-20251016-T00030
Please refer to the end of the specification for access instructions.

Lengthy table referenced here
US20250320519A1-20251016-T00031
Please refer to the end of the specification for access instructions.

Lengthy table referenced here
US20250320519A1-20251016-T00032
Please refer to the end of the specification for access instructions.

Lengthy table referenced here
US20250320519A1-20251016-T00033
Please refer to the end of the specification for access instructions.

Lengthy table referenced here
US20250320519A1-20251016-T00034
Please refer to the end of the specification for access instructions.

Lengthy table referenced here
US20250320519A1-20251016-T00035
Please refer to the end of the specification for access instructions.

Lengthy table referenced here
US20250320519A1-20251016-T00036
Please refer to the end of the specification for access instructions.

Lengthy table referenced here
US20250320519A1-20251016-T00037
Please refer to the end of the specification for access instructions.

Lengthy table referenced here
US20250320519A1-20251016-T00038
Please refer to the end of the specification for access instructions.

Lengthy table referenced here
US20250320519A1-20251016-T00039
Please refer to the end of the specification for access instructions.

Lengthy table referenced here
US20250320519A1-20251016-T00040
Please refer to the end of the specification for access instructions.

Lengthy table referenced here
US20250320519A1-20251016-T00041
Please refer to the end of the specification for access instructions.

Lengthy table referenced here
US20250320519A1-20251016-T00042
Please refer to the end of the specification for access instructions.

Lengthy table referenced here
US20250320519A1-20251016-T00043
Please refer to the end of the specification for access instructions.

Lengthy table referenced here
US20250320519A1-20251016-T00044
Please refer to the end of the specification for access instructions.

Lengthy table referenced here
US20250320519A1-20251016-T00045
Please refer to the end of the specification for access instructions.

Lengthy table referenced here
US20250320519A1-20251016-T00046
Please refer to the end of the specification for access instructions.

Lengthy table referenced here
US20250320519A1-20251016-T00047
Please refer to the end of the specification for access instructions.

Lengthy table referenced here
US20250320519A1-20251016-T00048
Please refer to the end of the specification for access instructions.

Lengthy table referenced here
US20250320519A1-20251016-T00049
Please refer to the end of the specification for access instructions.

Lengthy table referenced here
US20250320519A1-20251016-T00050
Please refer to the end of the specification for access instructions.

Lengthy table referenced here
US20250320519A1-20251016-T00051
Please refer to the end of the specification for access instructions.

Lengthy table referenced here
US20250320519A1-20251016-T00052
Please refer to the end of the specification for access instructions.

Lengthy table referenced here
US20250320519A1-20251016-T00053
Please refer to the end of the specification for access instructions.

Lengthy table referenced here
US20250320519A1-20251016-T00054
Please refer to the end of the specification for access instructions.

Lengthy table referenced here
US20250320519A1-20251016-T00055
Please refer to the end of the specification for access instructions.

Lengthy table referenced here
US20250320519A1-20251016-T00056
Please refer to the end of the specification for access instructions.

Lengthy table referenced here
US20250320519A1-20251016-T00057
Please refer to the end of the specification for access instructions.

Lengthy table referenced here
US20250320519A1-20251016-T00058
Please refer to the end of the specification for access instructions.

Lengthy table referenced here
US20250320519A1-20251016-T00059
Please refer to the end of the specification for access instructions.

Lengthy table referenced here
US20250320519A1-20251016-T00060
Please refer to the end of the specification for access instructions.

Lengthy table referenced here
US20250320519A1-20251016-T00061
Please refer to the end of the specification for access instructions.

Lengthy table referenced here
US20250320519A1-20251016-T00062
Please refer to the end of the specification for access instructions.

Lengthy table referenced here
US20250320519A1-20251016-T00063
Please refer to the end of the specification for access instructions.

Lengthy table referenced here
US20250320519A1-20251016-T00064
Please refer to the end of the specification for access instructions.

Lengthy table referenced here
US20250320519A1-20251016-T00065
Please refer to the end of the specification for access instructions.

Lengthy table referenced here
US20250320519A1-20251016-T00066
Please refer to the end of the specification for access instructions.

Lengthy table referenced here
US20250320519A1-20251016-T00067
Please refer to the end of the specification for access instructions.

Lengthy table referenced here
US20250320519A1-20251016-T00068
Please refer to the end of the specification for access instructions.

Lengthy table referenced here
US20250320519A1-20251016-T00069
Please refer to the end of the specification for access instructions.

Lengthy table referenced here
US20250320519A1-20251016-T00070
Please refer to the end of the specification for access instructions.

Lengthy table referenced here
US20250320519A1-20251016-T00071
Please refer to the end of the specification for access instructions.

Lengthy table referenced here
US20250320519A1-20251016-T00072
Please refer to the end of the specification for access instructions.

Lengthy table referenced here
US20250320519A1-20251016-T00073
Please refer to the end of the specification for access instructions.

Lengthy table referenced here
US20250320519A1-20251016-T00074
Please refer to the end of the specification for access instructions.

Lengthy table referenced here
US20250320519A1-20251016-T00075
Please refer to the end of the specification for access instructions.

Lengthy table referenced here
US20250320519A1-20251016-T00076
Please refer to the end of the specification for access instructions.

Lengthy table referenced here
US20250320519A1-20251016-T00077
Please refer to the end of the specification for access instructions.

Lengthy table referenced here
US20250320519A1-20251016-T00078
Please refer to the end of the specification for access instructions.

Lengthy table referenced here
US20250320519A1-20251016-T00079
Please refer to the end of the specification for access instructions.

Lengthy table referenced here
US20250320519A1-20251016-T00080
Please refer to the end of the specification for access instructions.

Lengthy table referenced here
US20250320519A1-20251016-T00081
Please refer to the end of the specification for access instructions.

Lengthy table referenced here
US20250320519A1-20251016-T00082
Please refer to the end of the specification for access instructions.

Lengthy table referenced here
US20250320519A1-20251016-T00083
Please refer to the end of the specification for access instructions.

Lengthy table referenced here
US20250320519A1-20251016-T00084
Please refer to the end of the specification for access instructions.

Lengthy table referenced here
US20250320519A1-20251016-T00085
Please refer to the end of the specification for access instructions.

Lengthy table referenced here
US20250320519A1-20251016-T00086
Please refer to the end of the specification for access instructions.

Lengthy table referenced here
US20250320519A1-20251016-T00087
Please refer to the end of the specification for access instructions.

Lengthy table referenced here
US20250320519A1-20251016-T00088
Please refer to the end of the specification for access instructions.

Lengthy table referenced here
US20250320519A1-20251016-T00089
Please refer to the end of the specification for access instructions.

Lengthy table referenced here
US20250320519A1-20251016-T00090
Please refer to the end of the specification for access instructions.

Lengthy table referenced here
US20250320519A1-20251016-T00091
Please refer to the end of the specification for access instructions.

Lengthy table referenced here
US20250320519A1-20251016-T00092
Please refer to the end of the specification for access instructions.

Lengthy table referenced here
US20250320519A1-20251016-T00093
Please refer to the end of the specification for access instructions.

Lengthy table referenced here
US20250320519A1-20251016-T00094
Please refer to the end of the specification for access instructions.

Lengthy table referenced here
US20250320519A1-20251016-T00095
Please refer to the end of the specification for access instructions.

Lengthy table referenced here
US20250320519A1-20251016-T00096
Please refer to the end of the specification for access instructions.

Lengthy table referenced here
US20250320519A1-20251016-T00097
Please refer to the end of the specification for access instructions.

Lengthy table referenced here
US20250320519A1-20251016-T00098
Please refer to the end of the specification for access instructions.

Lengthy table referenced here
US20250320519A1-20251016-T00099
Please refer to the end of the specification for access instructions.

Lengthy table referenced here
US20250320519A1-20251016-T00100
Please refer to the end of the specification for access instructions.

Lengthy table referenced here
US20250320519A1-20251016-T00101
Please refer to the end of the specification for access instructions.

Lengthy table referenced here
US20250320519A1-20251016-T00102
Please refer to the end of the specification for access instructions.

Lengthy table referenced here
US20250320519A1-20251016-T00103
Please refer to the end of the specification for access instructions.

Lengthy table referenced here
US20250320519A1-20251016-T00104
Please refer to the end of the specification for access instructions.

Lengthy table referenced here
US20250320519A1-20251016-T00105
Please refer to the end of the specification for access instructions.

Lengthy table referenced here
US20250320519A1-20251016-T00106
Please refer to the end of the specification for access instructions.

Lengthy table referenced here
US20250320519A1-20251016-T00107
Please refer to the end of the specification for access instructions.

Lengthy table referenced here
US20250320519A1-20251016-T00108
Please refer to the end of the specification for access instructions.

Lengthy table referenced here
US20250320519A1-20251016-T00109
Please refer to the end of the specification for access instructions.

Lengthy table referenced here
US20250320519A1-20251016-T00110
Please refer to the end of the specification for access instructions.

Lengthy table referenced here
US20250320519A1-20251016-T00111
Please refer to the end of the specification for access instructions.

Lengthy table referenced here
US20250320519A1-20251016-T00112
Please refer to the end of the specification for access instructions.

Lengthy table referenced here
US20250320519A1-20251016-T00113
Please refer to the end of the specification for access instructions.

### 8.11. Examples

### 8.11.1. Example 1—Method of Identifying IRES-Like Sequences

Based on the unique circular RNA reporter gene system of the present invention (the circular RNA reporter gene can be expressed under the control of the translation initiation element to produce green fluorescent protein), a set of libraries containing millions of different sequences were constructed, and different cell populations were transfected and screened by flow cytometry (negative: no green fluorescence, positive: green fluorescence with different intensities). Amplicon sequencing on collected different cell populations was performed. The sequence information contained in negative and different positive cells was analyzed in combination with computational biology analysis. Sequence features of different lengths from these sequence information was extracted. Based on these negative and positive sequence features, and through anti-neural network learning, translation initiation element modules of non-viral origin were generated.

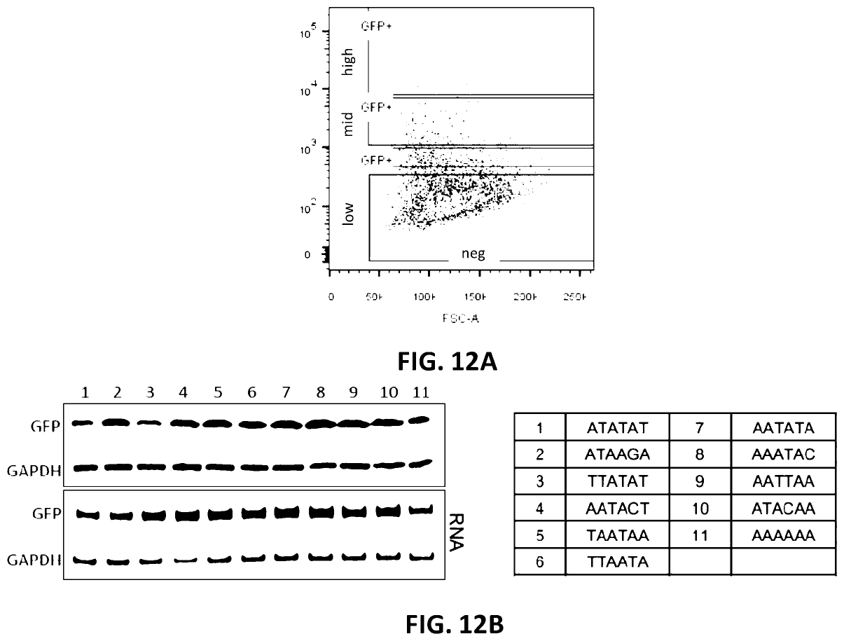

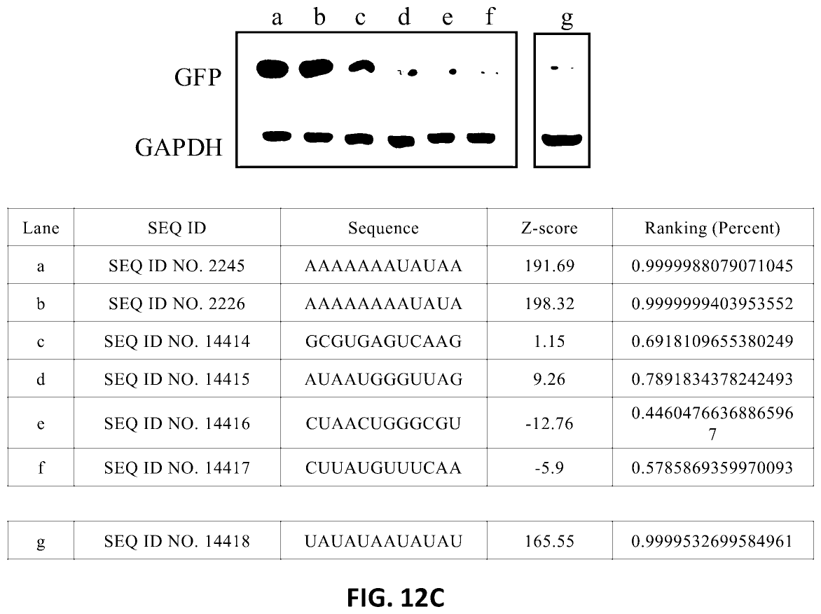

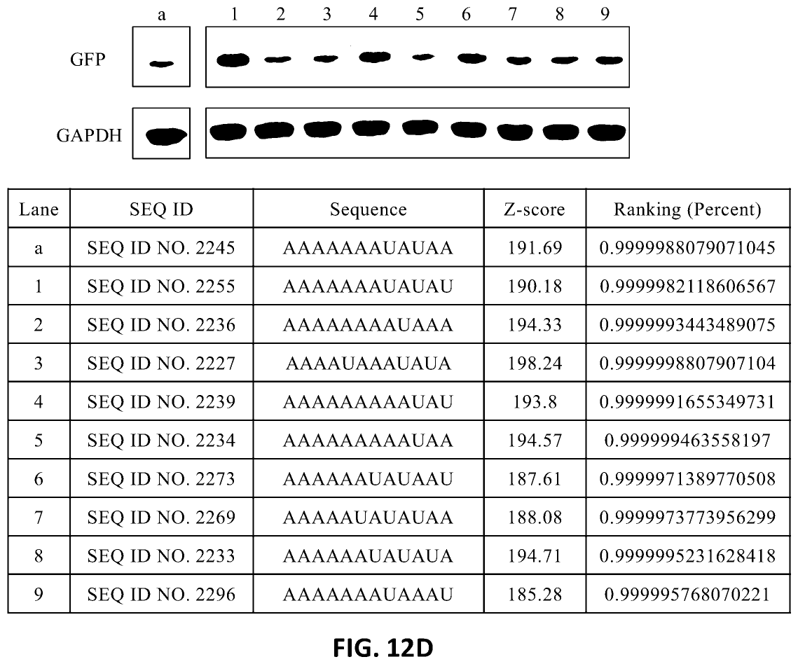

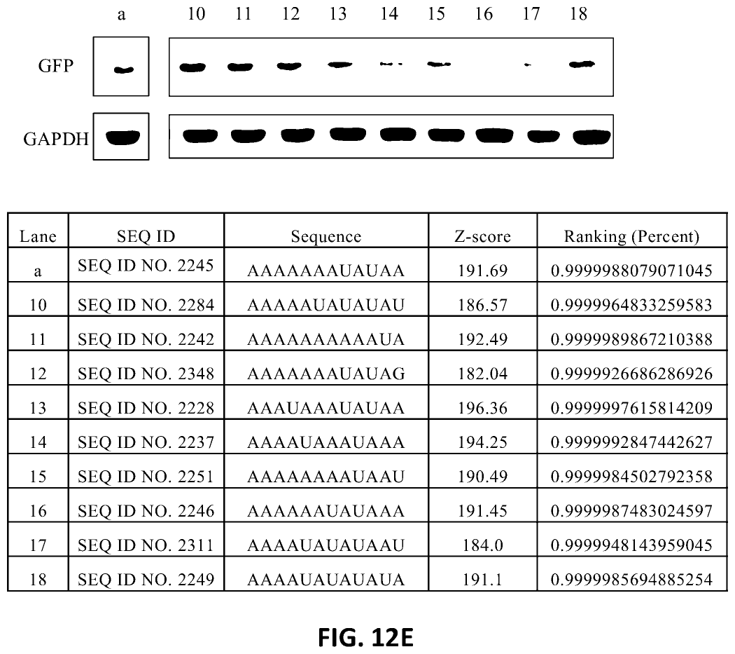

The translation initiation element modules and modular combination elements were inserted into circular RNA expression vectors, and the activity of translation initiation elements was detected by cell transfection and western blot. The high-throughput screening system based on circular RNA separated cell populations with different green fluorescence intensities (positive) and cell populations without fluorescence (negative). The results are shown in FIG. 12A. The results show that the circular RNA system can express green fluorescent protein and be used for screening. At the same time, the system can isolate cell populations with different fluorescence intensities, indicating that different inserted sequences in the library have a differential effect on the translation initiation of circular RNA.

Computational biology analysis was conducted using the amplicon sequencing data of different cell populations with different green fluorescence intensities (positive) and cell populations without fluorescence (negative).

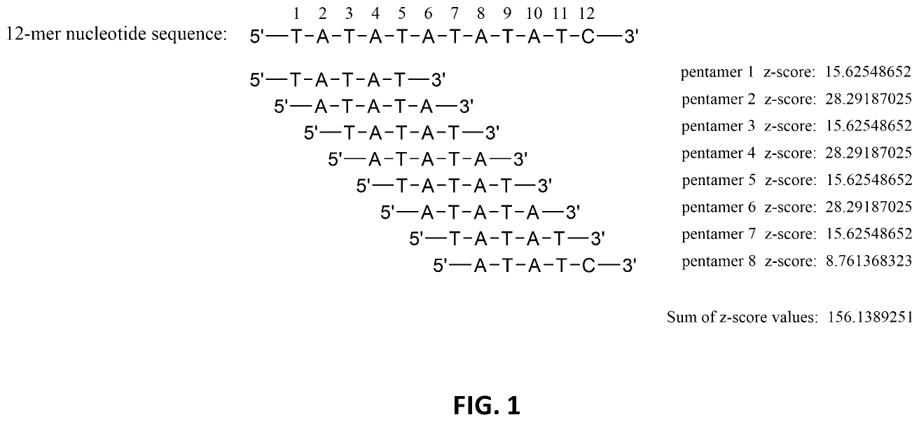

An exemplary RNA polynucleotide query sequence was generated using the method as described above (e.g., 12-mer nucleotide sequence) (FIG. 1). Overlapping polynucleotide fragment sequences within the polynucleotide query sequence were generated (e.g., pentamers 1-8). An enrichment score for each polynucleotide fragment sequence was generated (e.g., z-scores of pentamers 1-8) according to the following system of equations:

$$\begin{matrix}Z - Score = \frac{{f}_{1} - {f}_{2}}{\sqrt{( \frac{1}{{N}_{1}} + \frac{1}{{N}_{2}} ) P ( 1 - P )}} \\ P = \begin{matrix}{N}_{1} {f}_{1} - {N}_{2} {f}_{2} \\ {N}_{1} \vert {N}_{2}\end{matrix}\end{matrix}$$

wherein f₁ is the frequency of a certain sequence in a population with positive fluorescence intensities (i.e., the first population), f₂ is the frequency of the same sequence in population without fluorescence (i.e., the second population), N₁ is the size of population with positive fluorescence intensities (i.e., the first population), and N₂ is the size of population without fluorescence (i.e., the second population).

A numerical score for the polynucleotide query sequence was then generated by summing the enrichment scores of each polynucleotide fragment sequence (e.g., sum of Z-score values). The polynucleotide query sequence was identified as an IRES-like sequence when the numerical score for the polynucleotide query sequence was above a determined threshold.

The numerical scores were determined for polynucleotide query sequences that were 6-100 nucleotides in length (Tables 2-96, 110 and 112).

### 8.11.2. Example 2—IRES-Like Sequence-Based Vectors

Coding sequences of an expression protein with IRES-like sequence selected from Tables 101 and 102 were designed and used to construct circRNA vectors. RNAs were in vitro transcribed (“IVT”) from the XbaI digested linearized plasmid DNA template using T7 RiboMAX™ Large-Scale RNA Production System (Promega™, P1320) in the presence of unmodified NTPs. After DNase I treatment, the RNA products were column purified with RNA Clean and Concentrator Kit (ZYMO research, R1013) to remove excess NTP and other salt in IVT buffer, as well as the possible small RNA fragments generated during IVT. In some experiments, the purified RNA was further circularized in a new circularization buffer. The RNAs were first heated to 75° C. for 5 min and quickly cooled down to 45° C., after which a buffer including indicated magnesium and sodium was added to a final concentration: 50 mM Tris-HCl at pH 7.5, 50/100 mM NaCl, 0-40 mM MgCl2, and was then heated at 53° C. for indicated time for circularization. The best optimized reaction condition including concentration of magnesium and sodium, and incubation time at 53° C., was selected for further experiments.

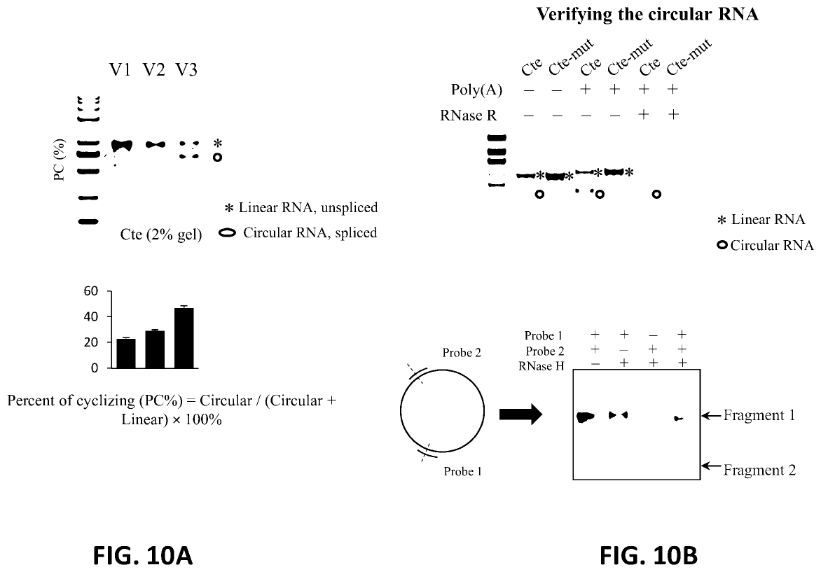

The production and purification of the resulting circRNAs were validated with (FIG. 10B).

### 8.11.3. Example 3—Protein Expression from Circular RNAs with IRES-Like Sequences

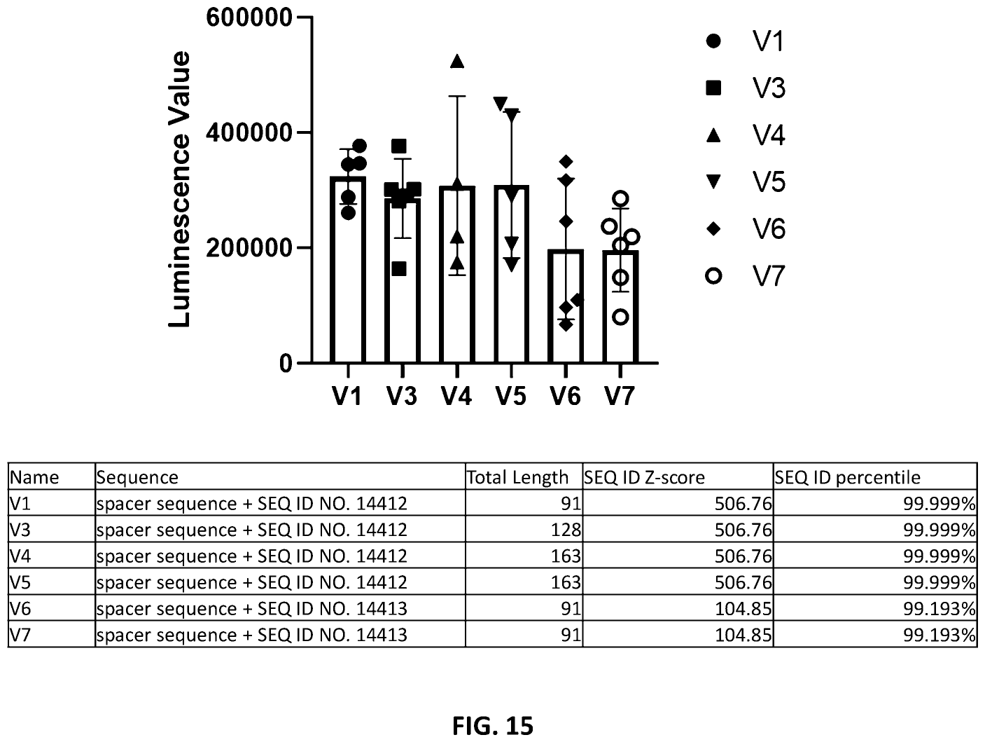

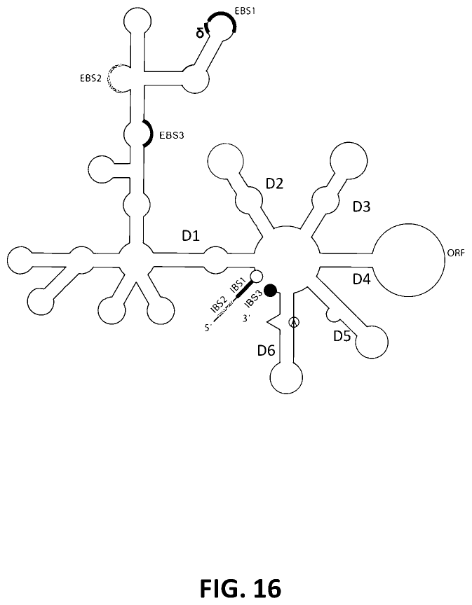

To test the function of the IRES-like sequences generated by the method described above, the DNA sequences encoding exemplary IRES-like sequences were cloned into a GFP expression vector (FIGS. 2, 12C, 12D and 12E) or luciferase expression vector (FIG. 15).

Cells were seeded into 24-well plate at one day before transfection. The purified circRNAs are transfected into cells using Lipofectamine Messenger Max (Invitrogen, LMRNA001) according to the manufacturer's manual. After transfection, cells were cultured at 37° C. for 24 h. The cell lysis and supernatant were collected for luminescence assay using Dual-Luciferase® Reporter Assay System (Promega™, E1910).

### Detection by Western Blot (GFP Expression)

The translation activity of the basic feature sequence of the translation initiation element was detected by cell transfection and western blot, and the result is shown for exemplary 12-mer polynucleotide sequences (FIGS. 2, 12C, 12D and 12E) and for exemplary 6-mer polynucleotide sequences (FIG. 12B). The results confirm that each translation elements have translation initiation functions, but different sequences (with different numerical scores, or percent rank) have different translation initiation capabilities (i.e., different level of protein and/or RNA expression). This indicates that based on these different sequence feature combinations, it is possible to obtain translation initiation elements with different activities. A higher Numerical score (z-score) corresponds with a higher level of protein expression by western blot.

### Detection by Luciferase Reporter Assay

Six exemplary circular RNAs were constructed (V1, V3, V4, V5, V6 and V7) and tested using the luciferase reporter assay described above. Each circular RNA comprises an IRES-like sequence and a spacer sequence (e.g., endogenous IRES sequence, linker elements, etc.). The identity of the IRES-like sequence, the numerical score of the IRES-like sequence and the percentile rank of the IRES-like sequence are shown in Table 111. The total length of the insert including both the IRES-like sequence and the various spacer sequences is also shown.

TABLE 111
Exemplary circular RNA for luciferase reporter assay

| | | | | Total length |
|---|---|---|---|---|
| | | | | of inserted |
| | | | | sequence in |
| Circular | IRES-like | Numerical | Percentile | expression |
| RNA | sequence | Score | Rank | vector |
| V1 | SEQ ID NO: 14412 | 506.76 | 99.999% | 91 |
| V3 | SEQ ID NO: 14412 | 506.76 | 99.999% | 128 |
| V4 | SEQ ID NO: 14412 | 506.76 | 99.999% | 163 |
| V5 | SEQ ID NO: 14412 | 506.76 | 99.999% | 163 |
| V6 | SEQ ID NO: 14413 | 104.85 | 99.193% | 91 |
| V7 | SEQ ID NO: 14413 | 104.85 | 99.193% | 91 |

V1, V3, V4 and V5 (with a higher numerical score and percentile rank) demonstrated a higher level of luminescence compared to V6 and V7 (lower numerical score and percentile rank) (FIG. 15). Together with the western blot assay results described above, it was shown that the use of different IRES-like sequences can be used to control the level of protein expression. The ability to fine-tune adjustments to the expression level (either higher or lower expression) by changing the identity of the IRES-like sequence in the circular RNA constructs is advantageous in therapeutic applications, where a specific threshold or range of protein expression must be met for functional activity. Further, the combination of two or more IRES-like sequences or the combination of an IRES-like sequence with spacer sequences (e.g., endogenous IRES sequence, linker elements, etc.) allow another advantageous mechanism for fine-tuning the expression level in therapeutic applications.

### 8.11.4. Example 4—Detection by Luciferase Reporter Assay

Five exemplary circular RNAs were constructed (S1, S2, S3, S4 and Gtx) and tested using the luciferase reporter assay described above. Each circular RNA comprises an IRES-like sequence of the present disclosure.

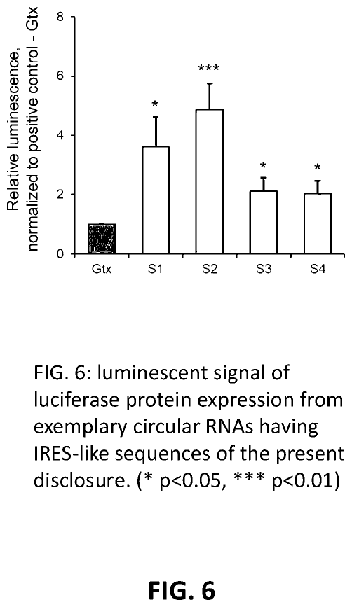

S1, S2, S3 and S4 demonstrated a higher level of luminescence compared Gtx (9-nt IRES module from the Gtx 5′ UTR that is 100% complementary to the 18S rRNA at nucleotides 1132-1124) (See FIG. 6).

### 8.11.5. Example 5—Methods of Improving IRES-Like Sequences

The IRES-like sequences identified via methods described herein (e.g., Section 8.2.2.2 and Example 1) were further optimized. As shown in the diagrams in FIG. 3 and FIG. 4, after the initial random sequence generation, Z-score evaluation, and/or sequence selection (FIG. 3 (100), (110), and (120) and FIG. 4 (200), (210), and (220)), additional steps were implemented to introduce mutation into the sequences (FIG. 3 (122) and FIG. 4 (222)), hybridization of sequences (FIG. 3 (121)), and/or crossover of sequences (FIG. 4 (221)). After such modification(s), the IRES-like sequences were evaluated for selection into the top sequence pool (FIG. 3 (130) and FIG. 4 (230)), discarded, or further optimized. Additional rounds of optimization, if needed, were performed.

### 8.11.6. Example 6. Screening of Group II Introns

This example relates to a method for determining the in vitro self-splicing capability of natural group II introns.

A DNA sequence according to the natural sequence of a group II intron was synthesized. The DNA sequence further comprised the naturally occurring flanking exon E1 and E2 sequences, including all or part of the intron binding region in the exon immediately adjacent to the group II intron. The DNA sequence was cloned into the expression vector psiCHECK-2 (Promega™, C8021) comprising the coding sequence of Rluc (Renilla Luciferase). psiCHECK-2 was digested with a single endonuclease, XhoI, and the DNA sequence was cloned into a vector, located 3′ downstream of Rluc, to obtain the corresponding construct. The skeleton of this expression vector was pcDNA3.1 comprising a T7 promoter and a T7 terminator.

PCR amplification was performed on the above vector using universal primers for T7 promoter and T7 terminator to obtain template DNAs for transcription. The PCR reaction conditions were: 95° C. for 30 s, 60° C. for 20 s, and 72° C. for 60 s, for 23 to 25 cycles. The template DNAs obtained were extracted with phenol-chloroform at a volume ratio of 1: 1, and then precipitated with 2.5 times by volume of absolute ethanol for purification.

The purified template DNAs were transcribed in vitro by T7 RNA polymerase. The transcripts were digested with DNase I at 37° C. for 30 min to degrade the PCR templates. The transcripts were then column purified to obtain high-purity RNAs.

The column-purified transcript RNAs were added to a self-splicing buffer (10, 20, 50 or 100 mM MgCl₂, 50 mM NaCl, 40 mM Tris-HCl, pH=7.5) for self-splicing reaction. The reaction conditions were: 95° C. for 1 min, 75° C. to 45° C. (−0.5° C., 15 sec/cycle, for 60 cycles in total), holding at 45° C. with a buffer added, 45° C. for 5 min, and 53° C. for 15 to 30 min. After the self-splicing reaction occurred in vitro, 200 ng of the product was used for electrophoresis analysis to detect the self-splicing efficiency of group II introns.

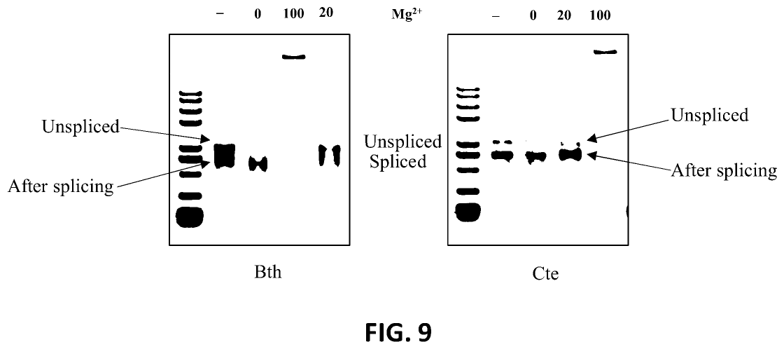

FIG. 9 shows the electrophoresis results of two group II introns identified by the above method that may perform self-splicing, namely the group II intron Bth from Bacillus thuringiensis and the group II intron Cte from Clostridium tetani. Unspliced RNAs (top row) and spliced RNAs (bottom row) were separated by electrophoresis. The group II intron and its flanking exon sequences (i.e., E1 and E2, each of 6 nucleotides, see Tables 103 and 104) confirmed by the method of this example were used as a precursor for the preparation of the self-splicing ribozyme cRNAzyme, also referred to as a cRNAzyme precursor.

### 8.11.7. Example 7. Preparation of Expression Constructs Comprising IRES-Like Sequence for Circular Construct

The self-splicing ribozyme cRNAzyme was further prepared from the group II intron cRNAzyme precursor, e.g., according to the method described in Chinese Patent Application No. 202110594352.4. The general principles for designing a cRNAzyme construct are that the total length of the intron sequence and the E1 and E2 sequences is as small as possible, and the cyclization rate is as high as possible. Exemplary sequences used in the preparation of expression constructs comprising IRES-like sequences for circular constructs is shown in Table 108.

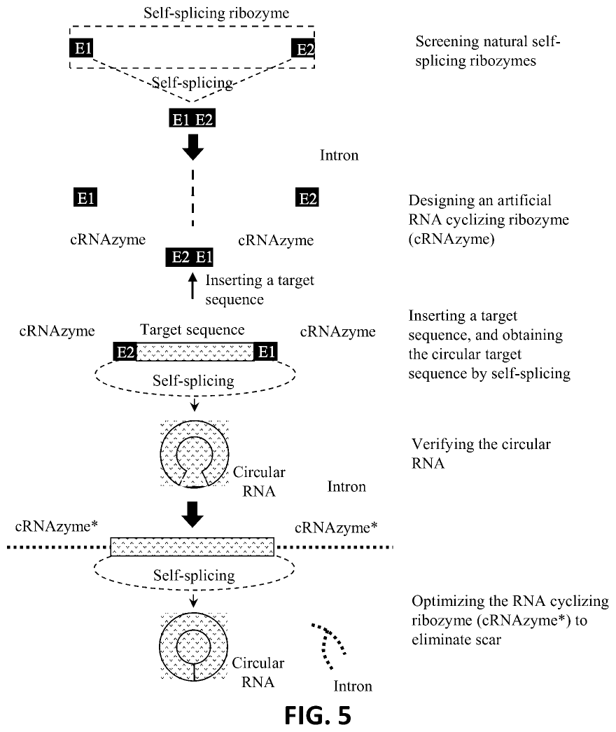

On the basis of the Cte cRNAzyme precursor sequence (SEQ ID NO: 14379, a total of 1,028 nucleotides in length, comprising the Cte intron itself and the exon sequences of 6 nucleotides at both ends, i.e., both E1 and E2 being 6 nucleotides in length), the intron-encoded protein (IEP) sequence of 310 nucleotides in domain 4 (nucleotide positions 625 to 934 of SEQ ID NO: 14379) was deleted, and the sequence that could fold correctly and maintain self-splicing activity was retained, comprising a few parts of exons at both ends (6 nt each, i.e., IBS1 and part of IBS3), thereby obtaining the E1-Cte^{Δ}^{^{IEP}}-E2 sequence. The E1-Cte^{Δ}^{^{IEP}}-E2 sequence was then bisected from a position inside the intron. The positions of the two fragments after segmentation were swapped, a first fragment consisting of E1 and a 5′ intron fragment was constructed to the 3′ end of the insert Rluc, and a second fragment consisting of a 3′ intron fragment and E2 was constructed to the 5′ end of the insert Rluc. On this basis, AATACCTTACTTAATAGTAACAATAGAAAATC (SEQ ID NO: 14391) was inserted at the 5′ end of the newly formed fragment, and AAGCTAGATCATATTACTATTAAGTAAGGTATT (SEQ ID NO: 14392) was inserted at the 3′ end, thereby obtaining a cRNAzyme_Cte construct. See FIG. 5. The two inserted sequences of SEQ ID NO: 14391 and SEQ ID NO: 14392 served as “homology arms” to make the 5′ and 3′ splice sites close to each other and improve the splicing efficiency. Three different segmentation positions were tried, which were located in loop regions in domain 1 (between positions 369 and 370), domain 3 (between positions 560 and 561), and domain 4 (between positions 825 and 826), respectively. Three cRNAzymes were thus formed, named cRNAzyme_Cte V1, cRNAzyme_Cte V2, and cRNAzyme_Cte V3, respectively. In the presence of cations, a self-splicing cyclization reaction was performed in vitro to test the cyclization activity of the obtained cRNAzyme. The splicing effects of the three cRNAzymes are shown in FIG. 10A.

It was determined that the RNA sequences spanned the E1E2 junction, which proved that E1 and E2 were linked together, and were circularized, as demonstrated by the methods below.

### Method I

The splice site in the Cte was mutated to lose its cyclization capability, as a linear RNA control of the same length but unable to achieve cyclization. Specifically, the splice site (nucleotides 1 to 26) in SEQ ID NO: 14379 was mutated to change C at position 3 to A, T at position 5 to G, G at position 17 to T, C at position 18 to T, A at position 21 to C, and T at position 26 to G. It would be understood by those skilled in the art that this mutation was intended to disrupt the cyclization capability, and other mutations in different numbers, positions, and types may also be made to achieve similar goal. The mutated Cte was referred to as Cte-mut. A polyA tail was then added to Cte-mut. Since a circular RNA was closed in a head-to-tail manner and had no 3′ end, it can not be poly-adenylated, as a linear RNA. The different in size after polyA addition can be resolved by electrophoresis.

The specific steps were as follows:
(1) The purified circular RNAs or control linear RNAs were tailed by poly(A) tailing enzyme (NEB), and the reaction conditions were at 37° C. for 30 min;
(2) RNase R (Lucigen) was added to digest the linear RNAs, and the reaction conditions were at 37° C. for 30 min; and
(3) RNAs were subjected to column purification.

As shown in the upper panel of FIG. 10B, lanes 3 and 4 were products with polyA tails added, while lanes 1 and 2 were products without polyA tails added. In the case of no cyclization, the bands of cRNAzymes based on Cte and Cte-mut all shifted upward after polyA addition reaction, indicating that the molecules became larger and polyA was successfully added. In contrast, the bands presumed to be circular RNAs were substantially at the same position and of the same size with and without the polyA addition reaction.

### Method II

Digestion was performed with RNase R. RNase R was a 3′-5′ exoribonuclease that may degrade linear RNA molecules de novo; and circular RNAs with closed loop structures can not be degraded. The digestion of RNAs may be identified by electrophoresis.

The specific steps were as follows:
(1) Through the annealing reaction, a DNA probe was bound to the RNA. The reaction was run at 95° C. for 2 min, and then slowly and gradually cooled to 25° C. The probe sequences were shown in SEQ ID NOs: 7 and 8.
(2) The DNA/RNA double strands were digested with RNase H (NEB), and the reaction conditions were at 37° C. for 30 min; and
(3) RNAs were subjected to column purification.

As shown in the upper panel of FIG. 10B, lanes 5 and 6 were the results after RNase R treatment, while lanes 1 to 4 were the results without RNase R treatment. It can be seen that the larger linear RNA bands disappeared after RNase R treatment (lanes 5 and 6), while the band presumed to be a circular RNA in lane 5 still existed.

### Method III

Digestion was performed with RNase H. RNase H is an endoribonuclease that may specifically hydrolyze the RNA in hybrid DNA-RNA strands. Since linear RNAs and circular RNAs had different structures, they may be cleaved into fragments of different lengths by RNase H after being bound to the same DNA probe. By agarose gel electrophoresis, the lengths of the RNA fragments produced by cleavage may be resolved, so as to infer the original structure of RNAs. Specifically, for circular RNAs, two DNA probes were used to bind to RNAs and then the product was cleaved, resulting in two bands. In contrast, if there was no cyclization, but still linearity, the same method should result in three bands. As shown in the lower panel of FIG. 10B, two bands were obtained for the product of the present application.

The results obtained by the above three methods all confirmed the successful formation of circular RNAs and verified the cyclization activity of the constructed cRNAzyme_Cte.

### 8.11.8. Example 8. Improvement of Self-Splicing Efficiency

### Optimization of Reaction Conditions

Combinations of various ion concentrations (50 mM and 100 mM NaCl; 2 mM, 5 mM, 10 mM, and 20 mM Mg²⁺) and various reaction times (5 min, 15 min, and 30 min) in the reaction system were tested to determine the optimal reaction system. The reaction system of 20 mM Mg²⁺ and 50 mM NaCl for 15 and 30 minutes significantly increased circularization.

### Sequence Optimization

The sequence was further engineered according to the RNA secondary structure. For example, the splicing efficiency was improved by incorporating spacer sequences in the target sequence to increase the flexibility of the structure, including, AT-rich sequences as spacer. Other methods or techniques known in the art can also be utilized.

### 8.11.9. Example 9. Preparation of “Scarless” Circular RNAs without Scar Sequences

The circular RNAs obtained using the aforementioned method comprise a few non-target sequences, i.e., sequences from exons E1 and E2. To remove these sequences, the construct may be further engineered.

When preparing the cRNAzyme construct, both ends of the target sequence were E2 and E1 with shorter lengths, which were derived from the intron binding sequences (IBS) of the flanking exon regions of the group II intron, respectively, and were generally between 0 to 20 nucleotides in length. With respect to forming circular RNA, although it may be undesirable to comprise sequences other than the target sequences, such as exon sequences E1 and E2, if E1 and E2 were removed directly, the self-splicing circularization process would be affected due to the lack of an IBS sequence that would interact with the EBS or δ sequence in the intron. Accordingly, a part of the target sequence was utilized as an “IBS” sequence, and the EBS or 6 in the intron was modified to interact with the region in the target sequence that is regarded as “IBS”. Such a method removed the dependence on E1 and E2, while ensuring that the cRNAzyme construct has the self-splicing function, and removes the exon sequences from the construct and the final circularization product. Exemplary cRNAzyme constructs with different target sequences (e.g., GFP, Gluc and 2A peptide) were constructed.

For example, based on the sequences of 6 nucleotides at both ends of each target sequence, the EBSI and upstream sequence of EBSI (including 6) in the group II intron were respectively replaced with sequences at least partially complementarily paired with the two 6-nucleotide sequences. Specifically, upstream sequence of EBS1 (including 6) was allowed to be complementarily paired with 6 nucleotides at the 5′ end of the target sequence in a linear state (such as the state prior to self-splicing circularization of the cRNAzyme construct), and EBSI was allowed to be complementarily paired with 6 nucleotides at the 3′ end of the target sequence in a linear state. Modified EBSI and upstream sequence of EBSI (including 6) were designed for three different target sequences. Notably, the engineered EBS1 and upstream sequence of EBS1 (including 6) did not have to be perfectly complementarily paired with the target sequence fragments serving as “IBS1” and “IBS3”. A certain percentage of mismatches, or a slightly less robust pairing of such as A and G, G and U may be allowed. In general, the EBS1 and upstream sequence of EBS1 (including 6) used to substitute the corresponding sequences in group II introns were complementary paired with the corresponding region in the target sequence on at least 60% of the nucleotide positions, or at least 60% identical to the complementary paired sequence of the corresponding region in the target sequence.

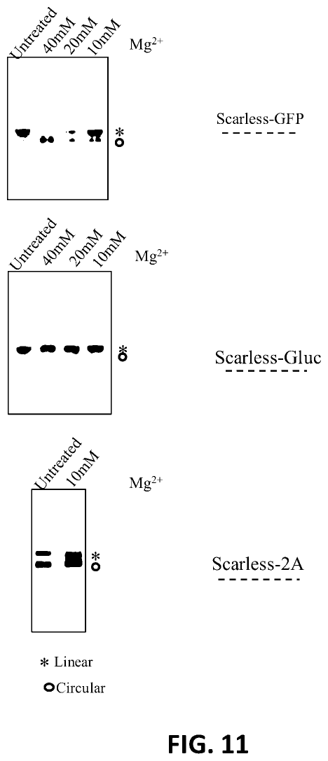

Circular RNAs were efficiently generated by this engineering method when different target sequences (GFP, Gluc, and 2A peptide) were used (FIG. 11). Sanger sequencing confirmed that such engineering might completely eliminate the exogenous scar sequence. After the circularization of the cRNAzyme construct with the EBS and upstream sequence of EBS1 (including 6) modified as described above, both ends of the target sequence (the two stretches of 6-nucleotide sequences, serving as “IBS1” and “IBS3”, respectively) were directly connected in a head-to-tail manner and the sequence was no longer separated by E1 and E2, which is more beneficial for subsequent applications of the generated circular RNAs. The constructs engineered in this way were referred to as “scarless” constructs, and the circular RNAs after circularization were referred to as “scarless” RNAs.

### 8.11.10. Example 10. Expression of the Therapeutic Product Using the Construct of the Present Disclosure

The expression of circular RNA products of different target sequences after transfection of cells was further tested. To minimize immunodegradation caused by linear RNAs, the RNA products were treated in the following three steps prior to transfection.
(1) RNase R treatment was performed to digest linear RNAs. The reaction conditions were at 37° C. for 30 min.
(2) CIP treatment was performed to remove phosphate groups at both ends of the linear RNAs. The reaction conditions were to add quick CIP (NEB), and react at 37° C. for 30 min.
(3) HPLC purification was performed to remove small linear RNAs. HPLC conditions: gel exclusion chromatographic column: Waters Xbridge® BEH450A, column temperature: 40° C., flow rate: 1 min/ml, and elution conditions: 0 to 30 min, 100% buffer A (prepared with 10 mM Tris, 0.5 mM EDTA, and DEPC-treated water).

The target RNAs were transfected using lipo RNAmax (Invitrogen®), under the transfection conditions according to the supplier's instructions, for 24 hours.

Using the construction methods described in the Examples above, different target sequences were tested. To facilitate detection of protein expression, constructs comprising a fluorescent protein coding sequence together with the IRES sequences disclosed herein were constructed. The addition of IRES may initiate cap-independent non-canonical translation, enabling the translation of coding sequences in circular RNAs into proteins.

For different target sequences, different methods were used to detect protein expression.

In the case that the translation product was GFP, the fluorescence was observed under a microscope. In addition, the cells were lysed with RIPA lysis solution (Beyotime), and then the protein expression was detected by Western blotting. In the case that the translation product was luciferase, the cells were first lysed with Passive lysis solution (Promega™) and then the protein expression was detected by a microplate reader using a luciferase detection kit (Promega™). It was confirmed that the expression of Gluc protein was obtained by the method of the present disclosure.

### 8.11.11. Example 11. Administration of circRNAs into Mice

The circular RNA was encapsulated in a lipid nanoparticle via the NanoAssemblr Ignite system as previously described (Corbett K. S. et al., Nature 586, 567 (2020), Polack F. P., et al., N. Engl. J. Med. 383, 2063 (2020). In brief, an aqueous solution of circRNA at pH 4.0 was rapidly mixed with a lipid mixture dissolved in ethanol, which contain different ionizable cationic lipid, distearoylphosphatidylcholine (DSPC), DMG-PEG2000, and cholesterol. The ratios for the lipid mixture were MC3:DSPC:Cholesterol:PEG-2000=50:10:38.5:1.5 for formulation 1, SM-102:DSPC:Cholesterol:PEG-2000=50:10:38.5:1.5 for formulation 2, and ALC-0315:DSPC:Cholesterol:ALC-0159=46.3:9.4:42.7:1.6 for formulation 3. The resulting LNP mixture was then dialyzed against PBS and stored at −80° C. at a concentration of 0.5 μg/μl for further application.

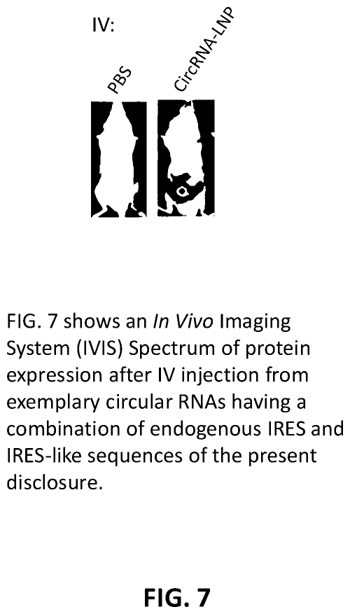

Female BALB/C mice aged 8 weeks were purchased from Shanghai Model Organisms Center. 20 μg of CircRNA-LNPs in PBS were administrated into mice intramuscularly with 3/10 insulin syringes (BD biosciences). The serum was collected 24 hours after the administration of the LNP, and 50 pl serum was used for Luciferase activity assay in vitro. Bioluminescence imaging was performed with an IVIS Spectrum (Roper Scientific). 24 hours after Gluc-LNP injection, 2 mg/kg of Coelenterazine (MedChemExpress, MCE) was administrated to mice intraperitoneally. Mice were then anesthetized after receiving the substrate in a chamber with 2.5% isoflurane (RWD Life Science Co.) and placed on the imaging platform while being maintained on 2% isoflurane via a nose cone. Mice were imaged 5 minutes post substrate injection with 30 seconds exposure time to ensure the signal were effectively and sufficiently acquired. The results were shown in FIG. 7.

For immunogenicity studies, 8-week-old female BALB/c mice (Shanghai Model Organisms Center, Inc) were used. 10 μg of CircRNA-RBD-LNP were diluted in 50 μl 1×PBS and intramuscularly administrated into the mice in the same hind leg for both prime and boost shots. Mice in the control groups received PBS or empty LNPs. The blood samples were collected 2 weeks after prime and boost shots. Mice spleen were also harvested at the end point (2 weeks after boost) for immunostaining and flow cytometry.

### 8.11.12. Example 12. Numerical Score Predictions of IRES-Like Sequences by Statistical Modeling

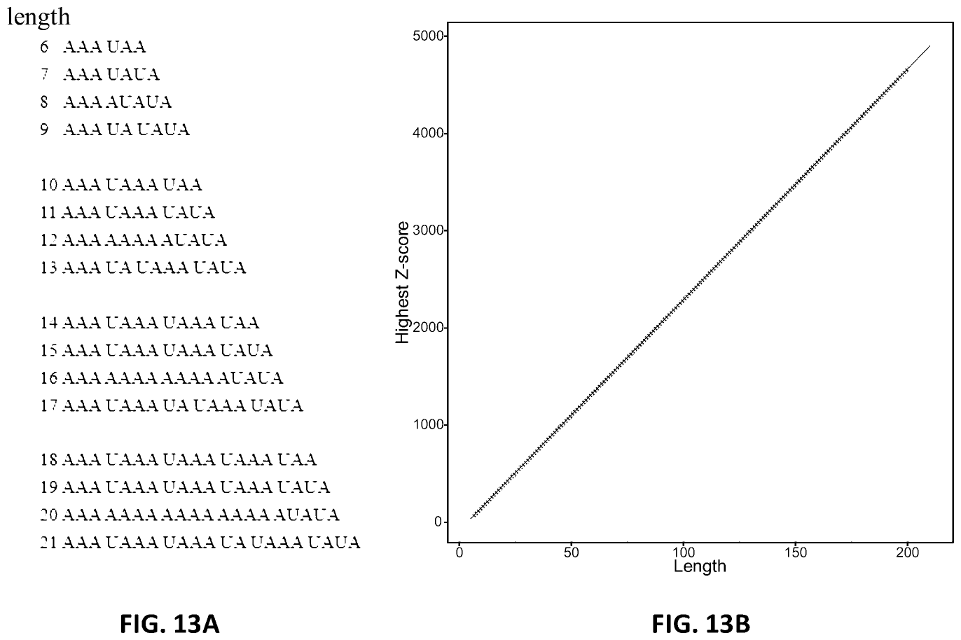

In order to predict the highest numerical scores (z-score) for any IRES polynucleotide sequence of any length, without having to generate each sequence permutation and calculating the score, the existing dataset from Example 1 was analyzed for patterns associated with highest numerical scores and a formula was determined for predicting the sequences with the highest numerical scores. The polynucleotide sequences with the highest calculated numerical scores for each total length were determined (FIG. 13A). For example, AAAUAA (SEQ ID NO: 1025) had the highest numerical score (56.73) for a polynucleotide with 6 residues in length (Table 2). AAAUAUA (SEQ ID NO: 1225) had the highest numerical score (84.25) for a polynucleotide with 7 residues in length (Table 3). AAAAUAUA (SEQ ID NO: 1425) had the highest numerical score (103.27) for a polynucleotide with 8 residues in length (Table 4). AAAUAUAUA (SEQ ID NO: 1425) had the highest numerical score (128.17) for a polynucleotide with 9 residues in length (Table 5). By identifying and reviewing the top polynucleotide sequences and numerical scores for those from 6 to 17 total nucleotides in length, certain rules and patterns were determined. For example, the first three residues of each sequence with the highest numerical score in its length category was AAA (FIG. 13A). After binning the first three residues into a segment, the remainder of the residues can be binned into groups of 3-5 nucleotides and the following formulas for binning the residues with the highest Z-score for each polynucleotide length was determined. n is an integer

For length ≥6 nt
Length is divisible by 4: AAA+n*AAAA+AUAUA
Length divided by 4 with a remainder of 1 (length=9): AAA+UA+UAUA
Length divided by 4 with a remainder of 1 (length=13): AAA+UA+UAAA+UAUA
Length divided by 4 with a remainder of 1 (length>13): AAA+n*UAAA+UA+UAAA+UAUA
Length divided by 4 with a remainder of 2: AAA+n*UAAA+UAA
Length divided by 4 with a remainder of 3: AAA+n*UAAA+UAUA

The above binning and formulas were used to predict the sequences with the highest numerical scores for poly nucleotides that were 18-21 residues in length and compared to the nucleotide permutations that were tested and calculated (Tables 14-17). The predicted sequence obtained from formula shown above was consistent with sequences that were tested. Next, the above formula was used to generate 10-200 nt highest score sequences and calculate their numerical scores, plotted in FIG. 13B. The scatter points represent the scores calculated and line is the fitted to the scatter points. Using linear fitting, the equation of the graph is: Highest Z-score=(23.74869×length of polynucleotide)−84.85243. The p-values of the slope and intercept are both less than 0.01. For example, using the equation, the highest Z-score for a 12-mer would be: (23.74869×12)−84.85243=200. As another example, the highest Z-score for a 100-mer would be (23.74869×100)−84.85243=2290.

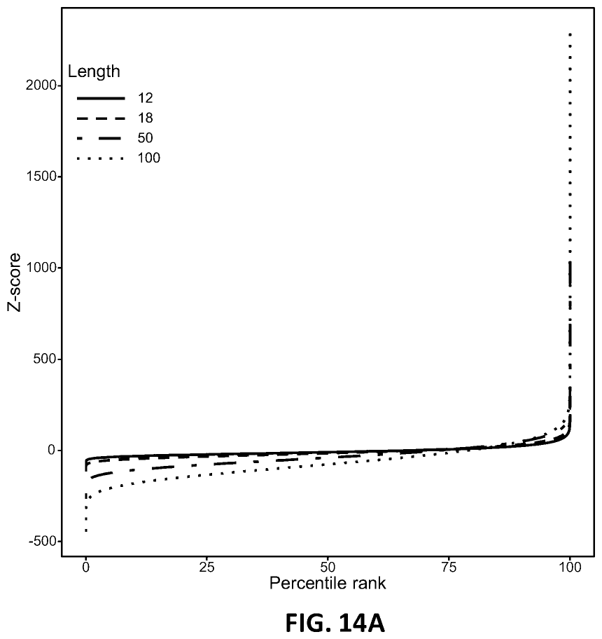

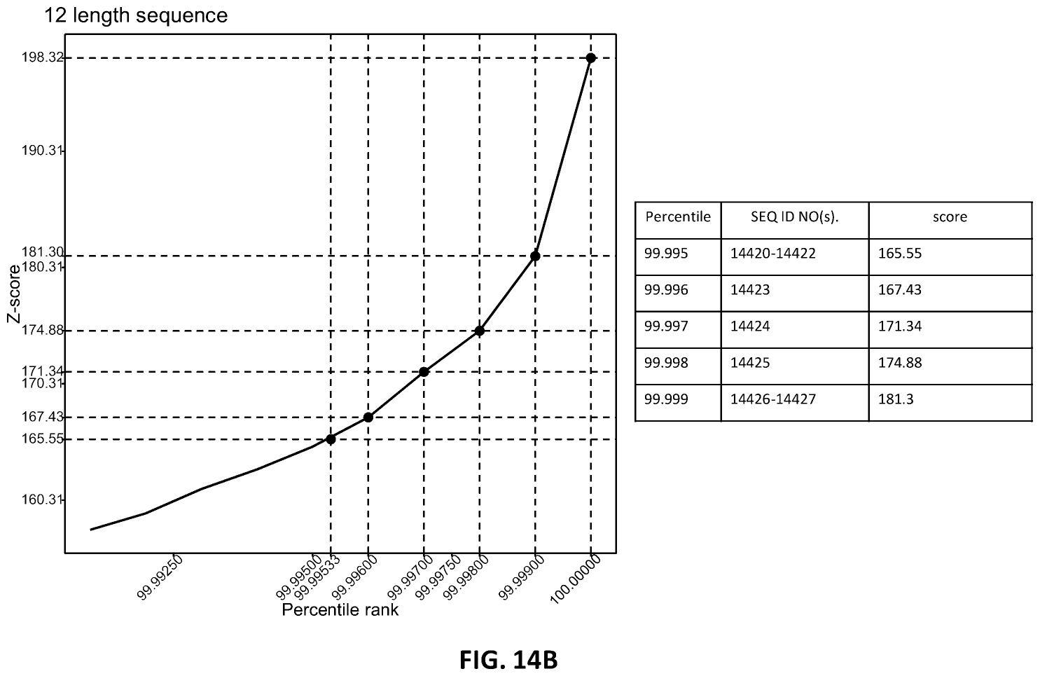

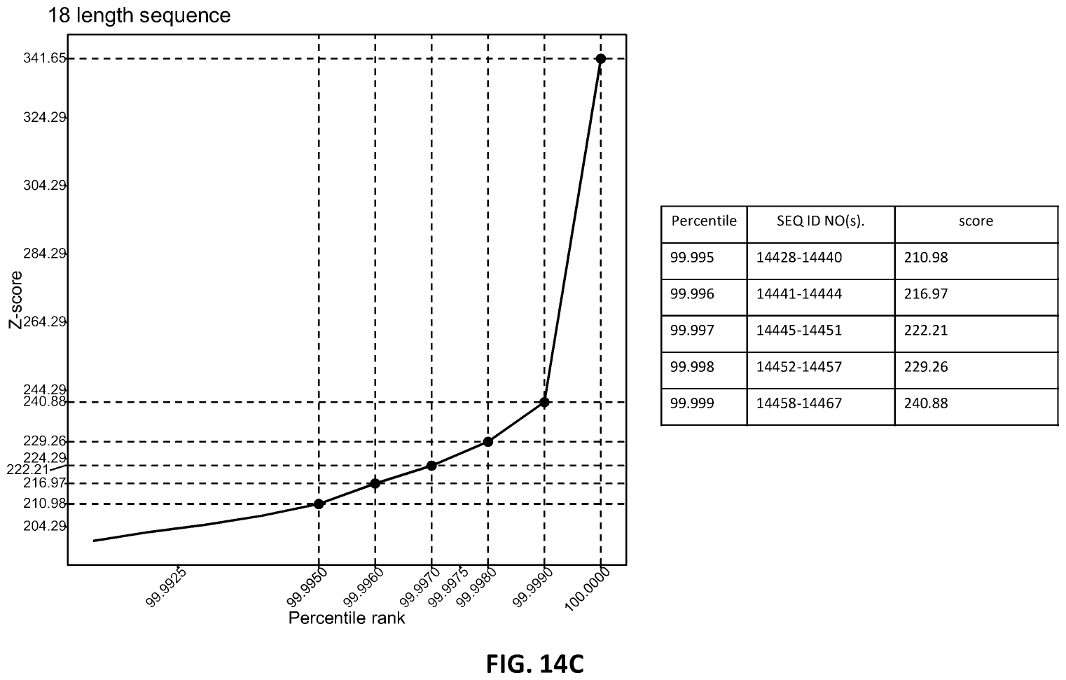

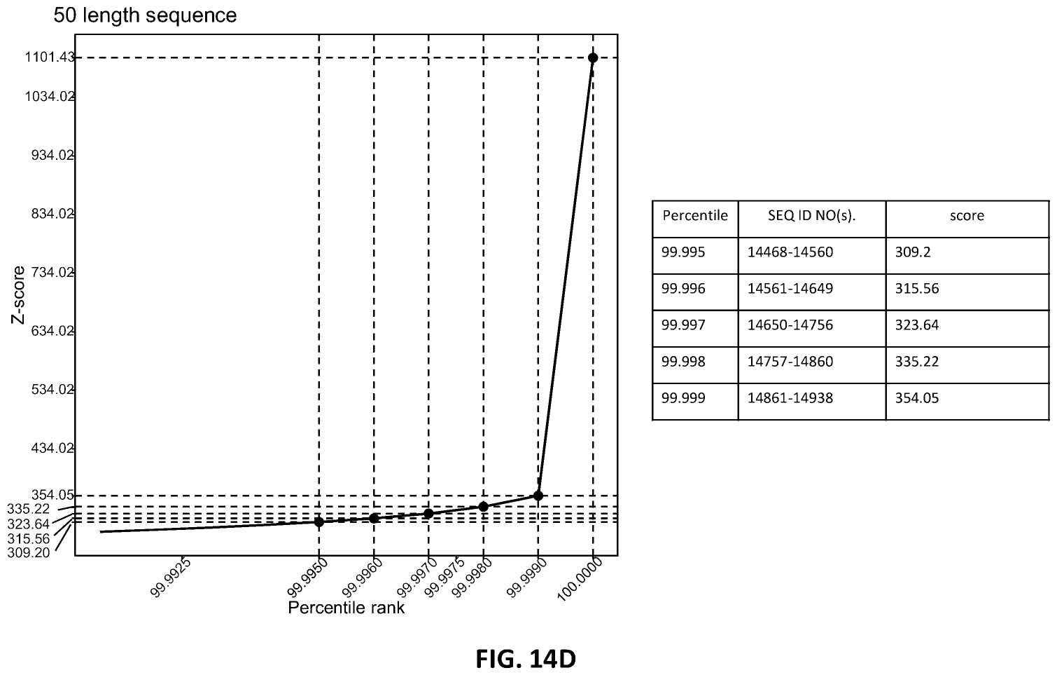

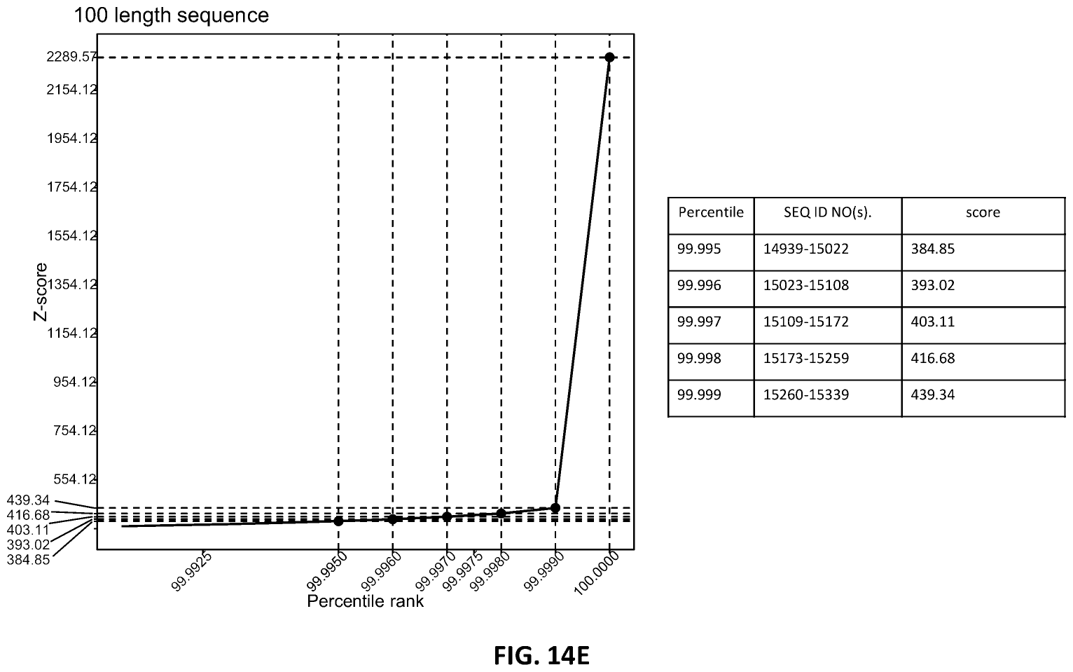

To further test these equations, each permutation of polynucleotides with 12, 18, 50 or 100 residues in length was generated and the numerical score was determined and ranked (percentile rank) (FIG. 14A). A zoomed in version of the graph is shown in FIGS. 14B, 14C, 14D and 14E, where the points represent the calculated numerical scores. For example, AAAAAAAAUAUA (SEQ ID NO: 2226) is shown with a numerical of 198.32. Thus, it was confirmed that that the linear fitting equation was accurate for predicting the highest numerical scores for each length category.

The combination of the formulas determined by sequence pattern analysis and the linear equation for predicting numerical scores is advantageous for predicting the sequence and functionality for IRES-like sequences, without having to generate and test each permutation of the sequences at each length. This is advantageous for predicting novel IRES-like sequences that drive protein expression (highest numerical scores) in therapeutic applications.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. All such similar substitutes and modifications are deemed to be within the spirit, scope and concept of the disclosure as defined by the appended claims.

### 8.11.13. Example 13. circRNAs Mediate Prolonged Protein Production

A major advantage of circular mRNAs was their superb stability because of lacking the free ends, therefore the circRNA should have good shelf life for protein expression compare to linear counterparts. To directly test this, both linear and circular mRNA encoding the Gaussia luciferase (Gluc) were synthesized and stored parallelly in pure water at room temperature for different days before transfecting them into 293 T cells. The activity of the circRNA to direct protein translation was tested in comparison to that of the linear mRNA. The prolonged protein translation was expected for the circRNA.

### 8.11.14. Example 14. Purified circRNAs Direct Robust Translation of Target Proteins

The mRNA purity was found to be a key factor for the protein production and induction of innate immunity, as the removal of dsRNA by HPLC can eliminate immune activation and improves translation of linear nucleoside-modified mRNA (Kariko, K. et al., (2011). To examine if the circRNAs produced using the cRNAzymes disclosed herein can induce innate immune response and cell toxicity, the circRNAs can be purified with gel purification or HPLC and measured if the circRNAs can induce cellular immune response upon transfection of circRNAs by testing cell survival and production of RIG-I and IFN-B1.

### 8.11.15. Example 15. LNP Encapsulated circRNAs Direct Robust Protein Production in Mouse

An important question for the therapeutic application of circRNAs was whether the production of circRNAs can be scaled up reliably and how the reproducibility between different batch of production. To confirm the scalability of this system, the IVT and circularization reaction system can be expanded for 50 fold (from 20 1 into 1 ml), and high circularization efficiency was tested. The efficacy was expected to stay essentially unchanged while the total amount of RNA products reach 7.5 mg in a single reaction.

The circRNAs produced herein can be further encapsulated with lipid nanoparticles (LNP) to for their in vivo delivery. The circRNAs in the aqueous solution were packed by ionizable cationic lipids, which form a nanoparticle with other lipid components such as DMG-PEG2000 and Cholesterol (see methods), achieving a ˜95% encapsulate efficiency with effective diameter at −80 nm. Different ionizable cationic lipids can be tested in our formulation to encapsulate circRNAs that encode Gluc, and the resulting LNPs were into BALC/c mice through intramuscular (IM) or intraperitoneal (IP) injection (n=3 for each experimental group). Three formulations using different ionizable cationic lipids (e.g., MC3, SM-102 and ALC-0315) were tested in this experiment. The expressions of luciferase were assayed either using the luciferase luminescence assay with serum or bioluminescence imaging of the animals.

LENGTHY TABLES
The patent application contains a lengthy table section. A copy of the table is available in electronic form from the USPTO web site (https://seqdata.uspto.gov/?pageRequest=docDetail&DocID=US20250320519A1). An electronic copy of the table will also be available from the USPTO upon request and payment of the fee set forth in 37 CFR 1.19(b)(3).

What is claimed is:

1. An RNA polynucleotide comprising a construct of Formula I, Formula II, Formula III, Formula IV, or Formula V: 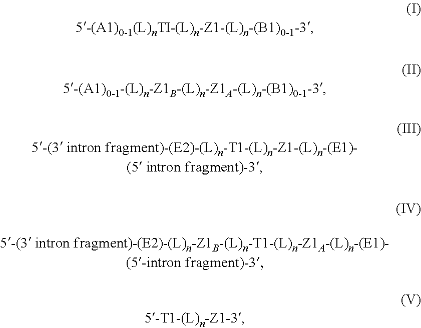
wherein:
TI is an engineered translation initiation element comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341;
Z1 is an expression sequence encoding a therapeutic product;
Z1_{A} is a first portion of an expression sequence encoding a therapeutic product;
Z1_{B} is a second portion of the expression sequence encoding the therapeutic product;
each L is independently a linker sequence;
A1 and B1 are each independently a sequences capable of circularizing the RNA polynucleotide, or
A1 and B1 each independently comprise a nucleotide derivative capable of joining the 5′ end and the 3′ end via a 3′ to 5′ phosphodiester linkage for circularization of the RNA polynucleotide;
the 5′ intron fragment and the 3′ intron fragment are each a fragment of a group II intron, wherein the 5′ intron fragment is located on the 5′ side of the 3′ intron fragment in the group II intron;
the E1 is a 5′ adjacent exon fragment of the group II intron, which is ≥0 nucleotides in length;
the E2 is a 3′ adjacent exon fragment of the group II intron, which is ≥0 nucleotides in length; and
n is an integer selected from 0 to 2.

2. The RNA polynucleotide of claim 1, further comprising a 5′ homology arm at the 5′ end of the 3′ intron fragment.

3. The RNA polynucleotide of claim 1, further comprising a 3′ homology arm at the 3′ end of the 5′ intron fragment.

4. The RNA polynucleotide of claim 1, further comprising a 5′ homology arm at the 5′ end of the 3′ intron fragment, and a 3′ homology arm at the 3′ end of the 5′ intron fragment.

5. The RNA polynucleotide of any one of claims 1-4, wherein the E1 and the E2 are each independently 0 to 20 nucleotides in length.

6. The RNA polynucleotide of any one of claims 1-5, wherein the 5′ intron fragment and the 3′ intron fragment are obtained by segmenting a group II intron at an unpaired region into two fragments, wherein the unpaired region is preferably selected from a linear region between two adjacent domains of the group II intron and a loop region of a stem-loop structure of domain 4 of the group II intron.

7. An RNA polynucleotide comprising an engineered translation initiation element (TI) comprising an IRES-like polynucleotide sequence, wherein the IRES-like polynucleotide sequence comprises the nucleic acid sequence of about or at least about 90%, 95%, 97%, 98%, 99% or 100% identical to SEQ ID NO: 1025-14161 or 14412-15341.

8. The RNA polynucleotide of any one of claims 1-7, wherein the IRES-like polynucleotide sequence is between 6-12 residues in length.

9. The RNA polynucleotide of any one of claims 1-8, wherein the TI further comprises a second IRES-like polynucleotide sequence.

10. The RNA polynucleotide of claim 9, wherein the second IRES-like polynucleotide sequence comprises the nucleic acid sequence of about or at least about 90%, 95%, 97%, 98%, 99% or 100% identical to SEQ ID NO: 1025-14161 or 14412-15341.

11. The RNA polynucleotide of any one of claims 1-6 and 8-10, wherein each L independently comprises a 5′UTR, 3′UTR, poly-A sequence, poly-A-C sequence, poly-C sequence, poly-U sequence, poly-G sequence, ribosome binding site, aptamer, riboswitch, ribozyme, small RNA binding site, translation regulation element (e.g., a Kozak sequence), protein binding site (e.g., PTBP1 or HUR), non-natural nucleotide, or non-nucleotide chemical-linker.

12. The RNA polynucleotide of any one of claims 1-6 and 8-11, wherein each L is independently about 3 to about 100 nucleotide residues in length.

13. The RNA polynucleotide of any one of claims 1-6 and 8-12, wherein each L independently comprises the nucleic acid sequence of RCC, wherein R is a guanine or an adenine.

14. The RNA polynucleotide of any one of claims 1-13, wherein the RNA polynucleotide is a single stranded RNA polynucleotide.

15. The RNA polynucleotide of any one of claims 1-14, wherein the RNA polynucleotide is a circular RNA polynucleotide.

16. The RNA polynucleotide of any one of claims 1-14, wherein the RNA polynucleotide is a linear RNA polynucleotide.

17. The RNA polynucleotide of any one of claims 1-14 and 16, wherein the RNA polynucleotide is capable of circularizing in the absence of an enzyme.

18. A polypeptide expressed by the RNA polynucleotide of any one of claims 1-17.

19. A DNA vector encoding the RNA polynucleotide of any one of claims 1-17.

20. A cell comprising the RNA polynucleotide of any one of claims 1-17, the polypeptide of claim 18, or the DNA vector of claim 19.

21. A composition comprising the RNA polynucleotide of any one of claims 1-17, the polypeptide of claim 18, the DNA vector of claim 19, or the cell of claim 20, and a pharmaceutically acceptable carrier.

22. A method of making a population of cells comprising contacting the cells of the population with the RNA polynucleotide of any one of claims 1-17, the polypeptide of claim 18, or the DNA vector of claim 19.

23. A method for generating an Internal Ribosome Entry Site (IRES)-like polynucleotide sequence, the method comprising the steps of:
(a) generating a polynucleotide query sequence consisting of X nucleic acid residues in length, wherein X is an integer greater than or equal to 3;
(b) generating X−Y+1 number of overlapping polynucleotide fragment sequences within the polynucleotide query sequence, wherein each polynucleotide fragment sequence consists of Y nucleic acid residues in length, wherein the first position of each polynucleotide fragment sequence is n and the last position of the same polynucleotide fragment sequence is Y+n−1, and wherein n represents each positive integer between 1 and X−Y+1;
(c) determining an enrichment score for each polynucleotide fragment sequence of (b);
(d) determining a numerical score for the polynucleotide query sequence by summing the enrichment scores of each polynucleotide fragment sequence of (c); and
(e) identifying the polynucleotide query sequence as an engineered IRES-like polynucleotide sequence according to a reference value.

24. The method of claim 23, wherein the reference value is calculated according to the following formula:
reference value=(23.75*X)−84.85−Z,
wherein Z represents any number between 1 and 10000 and X represents the length of IRES-like polynucleotide sequence.

25. The method of claim 24, wherein Z represents any number between 50 and 5000.

26. The method of claim 24, wherein Z represents any number between 175 and 2500.

27. The method of claim 24, wherein Z represents any number between 150 and 200.

28. The method of claim 23, wherein the reference value is characteristic of the absence of a therapeutic product expression.

29. The method of claim 23, wherein the reference value is the average score of all of the numerical scores of two or more IRES-like polynucleotide sequences or two or more natural IRES sequence or a combination thereof.

30. The method of claim 23, wherein the reference value is greater than or equal to 0.

31. The method of any one of claims 23-30, wherein X is an integer greater than or equal to 5.

32. The method of any one of claims 23-30, wherein X is an integer greater than or equal to 6.

33. The method of any one of claims 23-32, wherein the overlapping polynucleotide fragment sequences within the polynucleotide query sequence are 5, 6, 7, 8, 9 or 10 nucleic acid residues in length.

34. The method of any one of claims 23-33, wherein the enrichment score for a polynucleotide fragment sequence is determined by:
i) generating a expression plasmid library, wherein each expression plasmid of the library comprises a different polynucleotide fragment sequence and a reporter gene;
ii) contacting a population of cells with the expression plasmid library;
iii) quantifying the expression level of the reporter gene corresponding to each expression plasmid of the expression plasmid library;
iv) dividing the total population of cells into a first population and a second population based on the protein expression levels of iii);
v) determining an enrichment score for the polynucleotide fragment sequence using the following system of equations:
$$\begin{matrix}Score = \frac{{f}_{1} - {f}_{2}}{\sqrt{( \frac{1}{{N}_{1}} + \frac{1}{{N}_{2}} ) P ( 1 - P )}} \\ P = \frac{{N}_{1} {f}_{1} - {N}_{2} {f}_{2}}{{N}_{1} + {N}_{2}}\end{matrix}$$
wherein f₁ is the frequency of the polynucleotide fragment sequence in the first population,
wherein f₂ is the frequency of the same polynucleotide fragment sequence in the second population,
wherein N₁ is the size of the first population, and
wherein N₂ is the size of the second population.

35. The method of claim 34, wherein the first population has a protein expression level in the top 0-10 percent of protein expression levels of the total population and wherein the second population has a protein expression level in the bottom 10-90 percent of the protein expression levels of the total population.

36. The method of claim 34 or 35, wherein the first population has a protein expression level in the top 50.1 percent of protein expression levels of the total population and wherein the second population has a protein expression level in the bottom 49.9 percent of the protein expression levels of the total population.

37. The method of claim 34 or 35, wherein the first population has a protein expression level in the top 10 percent of protein expression levels of the total population and wherein the second population has a protein expression level in the bottom 90 percent of the protein expression levels of the total population.

38. The method of any one of claims 23-37, wherein the polynucleotide fragment sequence is 5 nucleic acid residues in length.

39. The method of claim 38, wherein the polynucleotide fragment sequence is selected from SEQ ID NO: 1-1024 with an enrichment score as shown in Table 1.

40. An RNA polynucleotide comprising an engineered translation initiation element (TI), wherein the TI comprises an Internal Ribosome Entry Site (IRES)-like polynucleotide sequence generated by the method of any one of claims 23-39.

41. A polypeptide expressed by the RNA polynucleotide of claim 40.

42. A DNA vector encoding the RNA polynucleotide of claim 40.

43. A cell comprising the RNA polynucleotide of claim 40, the polypeptide of claim 41, or the DNA vector of claim 42.

44. A composition comprising the RNA polynucleotide of claim 40, the polypeptide of claim 41, the DNA vector of claim 42, or the cell of claim 43, and a pharmaceutically acceptable carrier.

45. A method of making a population of cells comprising contacting the cells of the population with the RNA polynucleotide of claim 40, the polypeptide of claim 41, or the DNA vector of claim 42.

46. A method of modulating the expression of a protein in a subject in need thereof, comprising administering to the subject a therapeutically effective amount of the polynucleotide of any one of claims 1-17 and 40, the polypeptide of claim 18 or 41, the DNA vector of claim 19 or 42, or the cell of claim 20 or 43.

47. A method of treating or preventing a disease or disorder in a subject in need thereof, comprising administering to the subject a therapeutically effective amount of the polynucleotide of any one of claims 1-17 and 40, the polypeptide of claim 18 or 41, the DNA vector of claim 19 or 42, or the cell of claim 20 or 43.

48. Use of the polynucleotide of any one of claims 1-17 and 40, the polypeptide of claim 18 or 41, the DNA vector of claim 19 or 42, or the cell of claim 20 or 43 in the manufacture of a medicament for modulating the expression of a protein or treating or preventing a disease or disorder in a subject in need thereof.

49. A polynucleotide according to any one of claims 1-17 and 40, the polypeptide of claim 18 or 41, the DNA vector of claim 19 or 42, or the cell of claim 20 or 43, for modulating the expression of a protein or treating or preventing a disease or disorder in a subject in need thereof.
